(19) United States
(12) Patent Application Publication
(10) Pub. No.: US 2026/0356824 A1
(43) Pub. Date: Nov. 12, 2026

(54) COMPOUNDS FOR TREATING FIBROTIC DISEASES

(71) Applicant: Meta-Immue Inc., Siheung-si, Gyeonggi-do (KR)

(72) Inventors: Nakcheol Jeong, Hanam-si, Gyeonggi-do (KR); Petpiboon Prasit, Hanam-si, Gyeonggi-do (KR); Misun Lee, Hanam-si, Gyeonggi-do (KR); Jung-hee Kim, Hanam-si, Gyeonggi-do (KR)

(21) Appl. No.: 19/485,932

(22) PCT Filed: May 31, 2024
(86) PCT No.: PCT/US2024/032008

§ 371 Date: Nov. 19, 2025

Related U.S. Application Data
(60) Provisional application No. 63/470,603, filed on Jun. 2, 2023.

(51) Int. Cl. C07D 413/04; A61K 31/422; A61K 31/454; C07D 211/34; C07D 401/04; C07D 409/04; C07D 413/14

(52) U.S. Cl. CPC C07D 413/04; A61K 31/422; A61K 31/454; C07D 211/34; C07D 401/04; C07D 409/04; C07D 413/14

(57) ABSTRACT

This disclosure provides compounds and pharmaceutically acceptable salts thereof, that inhibit lysophosphatidic acid receptor 1 (LPA1). These compounds are useful, e.g., for treating a disease associated with LPA1 activity. This disclosure also provides compositions containing the same as well as methods of using and making the same.

### CROSS-REFERENCE TO RELATED APPLICATIONS

This application claims the benefit of priority to U.S. Application No. 63/470,603, filed on Jun. 2, 2024, the contents of which are hereby incorporated by reference.

### TECHNICAL FIELD

This disclosure relates to the fields of chemistry and biology. More particularly, this disclosure provides compounds and pharmaceutically acceptable salts thereof, that inhibit lysophosphatidic acid receptor 1 (LPA1). These compounds are useful, e.g., for treating a disease associated with LPA1 activity. This disclosure also provides compositions containing the same as well as methods of using and making the same.

### BACKGROUND

Lysophospholipids (LPAs) are bioactive lipids that regulate various cellular signaling pathways by binding to the 7-transmembrane domain G protein-coupled (GPCR) receptors. The LPAs have long been known as precursors of phospholipid biosynthesis in both eukaryotic and prokaryotic cells, but the LPAs have emerged only recently as signaling molecules that are rapidly produced and released by activated cells, notably platelets, to influence target cells by acting on specific cell-surface receptors. There are currently six identified LPA receptors designated as LPA1, LPA2, LPA3, LPA4, LPA5 and LPA6. These receptors signal through numerous effector pathways activated by heterotrimeric G proteins, including Gi/o, G12/13, Gq, and Gs. LPA receptor-mediated effects have been described in numerous cell types and model systems, both in vitro and in vivo, through gain- and loss-of-function studies. These studies have revealed physiological and pathophysiological influences on virtually every organ system and developmental stage of an organism. These include the nervous, cardiovascular, reproductive, and pulmonary systems. Disturbances in normal LPA signaling may contribute to a range of diseases, including neurodevelopmental and neuropsychiatric disorders, pain, cardiovascular disease, bone disorders, fibrosis, cancer, infertility, and obesity.

### SUMMARY

Some embodiments provide a compound of Formula (I), or a pharmaceutically acceptable salt thereof, as described herein. In some embodiments, the compound of Formula (I) is:

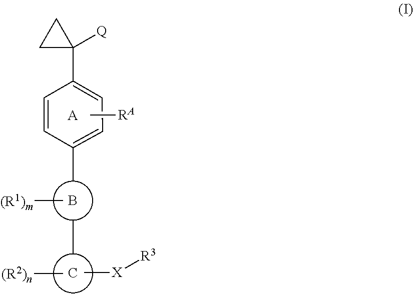

or pharmaceutically acceptable salts thereof, wherein:
R^{A} is hydrogen, C1-C6 alkyl, or C1-C6 alkoxy;
Ring B is 3-7 membered heterocyclylene or 5-6 membered heteroarylene;
Ring C is phenylene or 5-6 membered heteroarylene;
m is 0 or 1;
n is 0, 1, 2, or 3;
each R¹ is independently selected from halogen, C1-C6 alkyl, C1-C6 alkoxy, C1-C6 alkoxyalkyl, 3-7 membered heterocyclyl, and 5-6 membered heteroaryl; or two R¹ together with the atoms to which they are attached forms a 5-8 membered heterocyclyl or 5-6 membered heteroaryl;
each R² is independently selected from C1-C6 alkyl, C1-C6 haloalkyl, C1-C6 alkoxy, and halogen;
R³ is selected from:
(i) hydrogen,
(ii) C1-C6 alkyl optionally substituted with 1-2 independently selected R^{F},
(iii) C6-C10 aryl optionally substituted with hydroxyl,
(iv) C5-C10 cycloalkyl,
(v) 5-6 membered heteroaryl optionally substituted with phenyl, and
(vi) 5-6 membered heterocyclyl optionally substituted with phenyl;
each R^{E} is independently selected from:
(i) hydroxyl;
(ii) C1-C6 alkoxy,
(iii) phenyl optionally substituted with 1-3 substituents selected from C1-C6 alkyl, C1-C6 haloalkyl, C1-C6 alkoxyalkyl, C1-C6 alkoxy, hydroxyl, cyano, and halogen,
(iv) 5-10 membered heteroaryl optionally substituted with C1-C6 alkyl, and
(v) C3-C10 cycloalkyl;
Q is selected from:
(i) C1-C6 hydroxyalkyl,
(ii) —C(═O)OH,
(iii) —S(═O)₂OH,
(iv) cyano,
(v) —S(O)₂NR^{A1}R^{B1},
(vi) —NR^{C}S(O)₂NR^{A1}R^{B1},
(vii) —C(═O)C1-C6 alkoxy optionally substituted with C1-C6 alkoxy optionally substituted with —S(═O)₂NR^{C}R^{D}; or 5-6 membered heterocyclyl optionally substituted with C1-C6 alkyl;
(viii) —C(═O)C6-C10 aryloxy;
(ix) —C(═O)-(5-6 membered heterocyclyloxy) optionally substituted with 1-4 substituents selected from C1-C6 alkyl, hydroxyl, and —C(═O)OH,
(x) —C(═O)NR^{A2}R^{B2},
(xi) C1-C6 haloalkyl optionally substituted with hydroxyl,
(xii) 5-6 membered heteroaryl optionally substituted with hydroxyl, and
(xiii) —C(═O)NHS(═O)₂R^{F};
X is selected from —(CH₂)ₓNR^{G}C(═O)O—, —(CH₂)ₓOC(═O)NR^{G}—, —(CH₂)ₓNR^{G}—, or —CH(OH)—; R^{A1} and R^{B1} are independently selected from hydrogen and C1-C6 alkyl optionally substituted with hydroxyl;
R^{A2} and R^{B2} are independently selected from hydrogen and C1-C6 alkyl optionally substituted with hydroxyl or —S(═O)₂NH₂;
R^{C} and R^{D} are independently selected from hydrogen and C1-C6 alkyl;
R^{F} is selected from:
(i) C1-C6 alkyl optionally substituted with 1-3 substituents selected from:
   (a) hydroxyl,
   (b) halogen,
   (c) cyano,
   (d) C1-C6 alkoxy,
   (e) 4-6-membered heterocyclyl optionally substituted with C1-C6 alkyl,
   (f) —C(═O)OH,
   (g) —OC(═O)C1-C6 alkyl,
   (h) —S(═O)₂C1-C6 alkyl,
   (i) —C(═O)NR^{A1}R^{B1},
   (j) —NR^{C}R^{D},
   (k) —C(═O)C1-C6 aryloxy,
   (l) C3-C6 cycloalkyl optionally substituted with —C(═O)OH, C1-C6 hydroxyalkyl, —C(═O)NR^{A1}R^{B1}, or —C(═O)C1-C6 alkoxy, and
   (m) —C(═O)C1-C6 alkoxy optionally substituted with —C(═O)C1-C6 alkyl or —OC(═O)C1-C6 alkyl,
(ii) C2-C6 alkenyl,
(iii) C1-C6 haloalkyl,
(iv) phenyl,
(v) C3-C6 cycloalkyl optionally substituted with
   (a) C1-C6 alkyl optionally substituted with 1-3 substituents selected from hydroxyl, —C(═O)OH, —C(═O)NR^{A1}R^{B1}, or —C(═O)C1-C6 alkoxy,
   (b) cyano,
   (c) —C(═O)OH,
   (d) —C(═O)NR^{C}R^{D}, or€
   (e) —C(═O)C1-C6 alkoxy optionally substituted with hydroxyl or —C(═O)C1-C6 alkyl, or
   (f) —NR^{C}R^{D}, and
(vi) —NR^{A3}R^{B3};
R^{A3} and R^{B3} are independently selected from hydrogen and C1-C6 alkyl;
R^{G} is H or C1-C3 alkyl;
and x is 0 or 1.

Some embodiments provide a pharmaceutical composition comprising a compound of Formula (I), or a pharmaceutically acceptable salt thereof, and a pharmaceutically acceptable carrier, or a pharmaceutical composition as described herein.

Some embodiments provide a method of treating a fibrotic disease in a subject in need thereof, comprising administering to the subject a therapeutically effective amount of a compound of Formula (I), or a pharmaceutically acceptable salt thereof, or a pharmaceutical composition as described herein.

Some embodiments provide a method for reducing LPA1 activation in a cell comprising LPA1, the method comprising contacting the cell with a compound of Formula (I), or a pharmaceutically acceptable salt thereof.

The details of one or more embodiments of this disclosure are set forth in the accompanying drawings and the description below. Other features and advantages of the present disclosure will be apparent from the description and the claims.

### ADDITIONAL DEFINITIONS

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties. In case of conflict, the present specification, including definitions, will control.

The term “about” when referring to a number or a numerical range means that the number or numerical range referred to is an approximation, for example, within experimental variability and/or statistical experimental error, and thus the number or numerical range may vary up to ±10% of the stated number or numerical range.

The term “pharmaceutically acceptable carrier” means a pharmaceutically-acceptable material, composition, or vehicle, such as a liquid or solid filler, diluent, solvent, or encapsulating material. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salts not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “pharmaceutically acceptable excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The phrase “therapeutically effective amount” means an amount of compound that, when administered to a subject in need of such treatment, is sufficient to (i) treat a disease or disorder described herein (e.g., a fibrotic disease), (ii) attenuate, ameliorate, or eliminate one or more symptoms of the particular disease, condition, or disorder, or (iii) delay the onset of one or more symptoms of the particular disease, condition, or disorder described herein.

The term “halogen” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “hydroxyl” refers to an —OH radical.

The term “cyano” refers to a —CN radical.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “alkenyl” refers to an alkyl group having at least one double-bond between constituent carbon atoms. Non-limiting examples include ethenyl, n-propenyl, iso-propenyl, n-butenyl, sec-butenyl.

The term “haloalkyl” refers to an alkyl, in which one or more hydrogen atoms is/are replaced with an independently selected halogen.

The term “hydroxyalkyl” refers to an alkyl, in which one or more hydrogen atoms is/are replaced with hydroxyl.

The term “alkoxy” refers to an —O-alkyl radical (e.g., —OCH₃).

The term “alkoxyalkyl” refers to an alkyl, in which one or more hydrogen atoms is/are replaced with an independently selected alkoxy.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “aryloxy” refers to an —O-aryl radical (e.g., phenoxy).

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “heteroaryl”, as used herein, means a mono-, bi-, tri- or polycyclic group having 5 to 20 ring atoms, alternatively 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl, benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

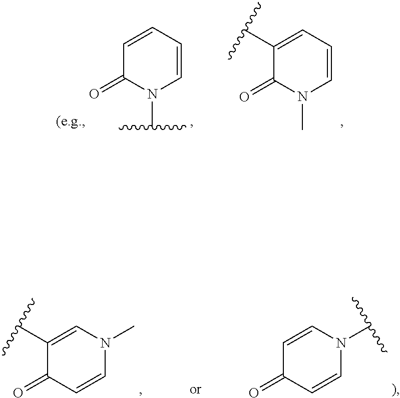

pyrimidone

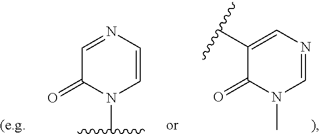

pyridazinone

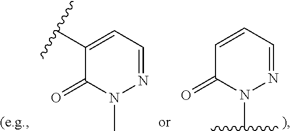

pyrazinone

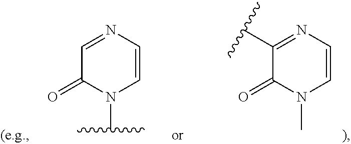

and imidazolone

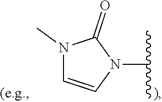 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated or partially unsaturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein one or more ring atoms may be substituted by 1-3 oxo (forming, e.g., a lactam) and one or more N or S atoms may be substituted by 1-2 oxido (forming, e.g., an N-oxide, an S-oxide, or an S,S-dioxide), valence permitting; and wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, examples of aromatic rings include: benzene, pyridine, pyrimidine, pyrazine, pyridazine, pyridone, pyrrole, pyrazole, oxazole, thiazole, isoxazole, isothiazole, and the like.

The term “heterocyclyloxy” refers to an —O-heterocylyl radical.

The term “saturated” as used in this context means only single bonds present between constituent atoms.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

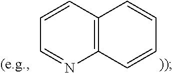

(ii) a single ring atom (spiro-fused ring systems)

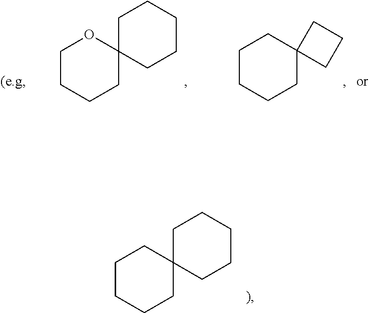 [SMILES: C1CCC2(CC1)CCCCO2 ; C1CCC2(CC1)CCC2 ; C1CCC2(CC1)CCCCC2]

or (iii) a contiguous array of ring atoms (bridged ring systems having all bridge lengths >0)

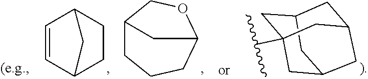

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

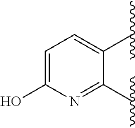 [SMILES: *c1ccc(O)nc1*]

encompasses the tautomeric form containing the moiety:

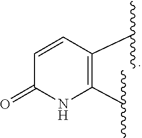 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The details of one or more embodiments of this disclosure are set forth in the accompanying drawings and the description below. Other features and advantages of the present disclosure will be apparent from the description and drawings, and from the claims.

### DETAILED DESCRIPTION

LPA signaling promotes normal wound healing and collagen deposition, including fibroblast activation, proliferation, and migration. However, increased LPA levels and activation of LPA1 can promote fibrosis, the result of an uncontrolled tissue healing process which leads to excessive accumulation and insufficient resorption of extracellular matrix (ECM) which ultimately results in end-organ failure.

This disclosure provides compounds of Formula (I), and pharmaceutically acceptable salts thereof, that are antagonists of LPA1. These chemical entities are useful, e.g., for treating a disease in which increased (e.g., excessive) LPA1 activation contributes to the pathology and/or symptoms and/or progression of the disease (e.g., a fibrotic disease in a subject (e.g., a human). This disclosure also provides compositions containing the same as well as methods of using and making the same.

### Formulae (I) Compounds

Some embodiments provide a compound of Formula (I):

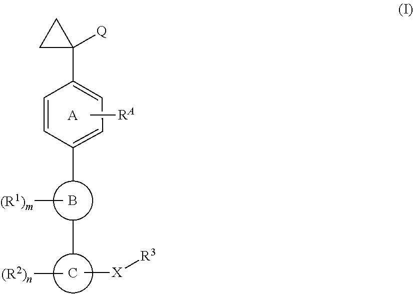

or pharmaceutically acceptable salts thereof, wherein:
R^{A} is hydrogen, C1-C6 alkyl, or C1-C6 alkoxy;
Ring B is 3-7 membered heterocyclylene or 5-6 membered heteroarylene;
Ring C is phenylene or 5-6 membered heteroarylene;
m is 0 or 1;
n is 0, 1, 2, or 3;
each R¹ is independently selected from halogen, C1-C6 alkyl, C1-C6 alkoxy, C1-C6 alkoxyalkyl, 3-7 membered heterocyclyl, and 5-6 membered heteroaryl; or two R¹ together with the atoms to which they are attached forms a 5-8 membered heterocyclyl or 5-6 membered heteroaryl;
each R² is independently selected from C1-C6 alkyl, C1-C6 haloalkyl, C1-C6 alkoxy, and halogen;
R³ is selected from:
(i) hydrogen,
(ii) C1-C6 alkyl optionally substituted with 1-2 independently selected R^{E},
(iii) C6-C10 aryl optionally substituted with hydroxyl,
(iv) C5-C10 cycloalkyl,
(v) 5-6 membered heteroaryl optionally substituted with phenyl, and
(vi) 5-6 membered heterocyclyl optionally substituted with phenyl;
each R^{E} is independently selected from:
(i) hydroxyl;
(ii) C1-C6 alkoxy,
(iii) phenyl optionally substituted with 1-3 substituents selected from C1-C6 alkyl, C1-C6 haloalkyl, C1-C6 alkoxyalkyl, C1-C6 alkoxy, hydroxyl, cyano, and halogen,
(iv) 5-10 membered heteroaryl optionally substituted with C1-C6 alkyl, and
(v) C3-C10 cycloalkyl;
Q is selected from:
(i) C1-C6 hydroxyalkyl,
(ii) —C(═O)OH,
(iii) —S(═O)₂OH,
(iv) cyano,
(v) —S(O)₂NR^{A1}R^{B1},
(vi) —NR^{C}S(O)₂NR^{A1}R^{B1},
(vii) —C(═O)C1-C6 alkoxy optionally substituted with C1-C6 alkoxy optionally substituted with —S(═O)₂NR^{C}R^{D}; or 5-6 membered heterocyclyl optionally substituted with C1-C6 alkyl;
(viii) —C(═O)C6-C10 aryloxy;
(ix) —C(═O)-(5-6 membered heterocyclyloxy) optionally substituted with 1-4 substituents selected from C1-C6 alkyl, hydroxyl, and —C(═O)OH,
(x) —C(═O)NR^{A2}R^{B2},
(xi) C1-C6 haloalkyl optionally substituted with hydroxyl,
(xii) 5-6 membered heteroaryl optionally substituted with hydroxyl, and
(xiii) —C(═O)NHS(═O)₂R^{F};
X is selected from —(CH₂)ₓNR^{G}C(═O)O—, —(CH₂)ₓOC(═O)NR^{G}—, —(CH₂)ₓNR^{G}, or —CH(OH)—; R^{A1} and R^{B1} are independently selected from hydrogen and C1-C6 alkyl optionally substituted with hydroxyl;
R^{A2} and R^{B2} are independently selected from hydrogen and C1-C6 alkyl optionally substituted with hydroxyl or —S(═O)₂NH₂;
R^{C} and R^{D} are independently selected from hydrogen and C1-C6 alkyl;
R^{F} is selected from:
(i) C1-C6 alkyl optionally substituted with 1-3 substituents selected from:
   (a) hydroxyl,
   (b) halogen,
   (c) cyano,
   (d) C1-C6 alkoxy,
   (e) 4-6-membered heterocyclyl optionally substituted with C1-C6 alkyl,
   (f) —C(═O)OH,
   (g) —OC(═O)C1-C6 alkyl,
   (h) —S(═O)₂C1-C6 alkyl,
   (i) —C(═O)NR^{A1}R^{B1},
   (j) —NR^{C}R^{D},
   (k) —C(═O)C1-C6 aryloxy,
   (l) C3-C6 cycloalkyl optionally substituted with —C(═O)OH, C1-C6 hydroxyalkyl, —C(═O)NR^{A1}R^{B1}, or —C(═O)C1-C6 alkoxy, and
   (m) —C(═O)C1-C6 alkoxy optionally substituted with —C(═O)C1-C6 alkyl or —OC(═O)C1-C6 alkyl,
(ii) C2-C6 alkenyl,
(iii) C1-C6 haloalkyl,
(iv) phenyl,
(v) C3-C6 cycloalkyl optionally substituted with
   (a) C1-C6 alkyl optionally substituted with 1-3 substituents selected from hydroxyl, —C(═O)OH, —C(═O)NR^{A1}R^{B1}, or —C(═O)C1-C6 alkoxy,
   (b) cyano,
   (c) —C(═O)OH,
   (d) —C(═O)NR^{C}R^{D}, or
   (e) —C(═O)C1-C6 alkoxy optionally substituted with hydroxyl or —C(═O)C1-C6 alkyl, or
   (f) —NR^{C}R^{D}, and
(vi) —NR^{A3}R^{B3};
R^{A3} and R^{B3} are independently selected from hydrogen and C1-C6 alkyl;
R^{G} is H or C1-C3 alkyl;
and x is 0 or 1.

In some embodiments, Formula (I) is Formula (I-I):

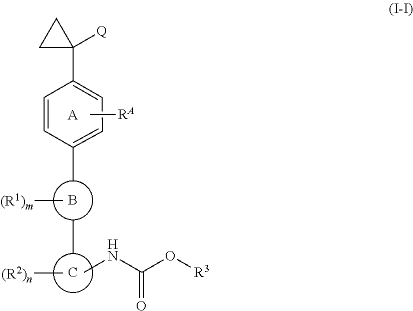

or pharmaceutically acceptable salts thereof, wherein:
R^{A} is hydrogen, C1-C6 alkyl, or C1-C6 alkoxy;
Ring B is 3-7 membered heterocyclylene or 5-6 membered heteroarylene;
Ring C is phenylene or 5-6 membered heteroarylene;
m is 0 or 1;
n is 0, 1, 2, or 3;
each R¹ is independently selected from halogen, C1-C6 alkyl, C1-C6 alkoxy, C1-C6 alkoxyalkyl, 3-7 membered heterocyclyl, and 5-6 membered heteroaryl; or two R¹ together with the atoms to which they are attached forms a 5-8 membered heterocyclyl or 5-6 membered heteroaryl;
each R² is independently selected from C1-C6 alkyl, C1-C6 haloalkyl, C1-C6 alkoxy, and halogen;
R³ is selected from
(i) hydrogen,
(ii) C1-C6 alkyl optionally substituted with 1-2 independently selected R^{E},
(iii) C6-C10 aryl optionally substituted with hydroxyl,
(iv) C5-C10 cycloalkyl,
(v) 5-6 membered heteroaryl optionally substituted with phenyl, and
(vi) 5-6 membered heterocyclyl optionally substituted with phenyl;
each R^{E} is independently selected from
(i) hydroxyl;
(ii) C1-C6 alkoxy,
(iii) phenyl optionally substituted with 1-3 substituents selected from C1-C6 alkyl, C1-C6 haloalkyl, C1-C6 alkoxyalkyl, C1-C6 alkoxy, hydroxyl, cyano, and halogen,
(iv) 5-10 membered heteroaryl optionally substituted with C1-C6 alkyl, and
(v) C3-C10 cycloalkyl;
Q is selected from:
(i) C1-C6 hydroxyalkyl,
(ii) —C(═O)OH,
(iii) —S(═O)₂OH,
(iv) cyano,
(v) —S(O)₂NR^{A1}R^{B1},
(vi) —NR^{C}S(O)₂NR^{A1}R^{B1},
(vii) —C(═O)C1-C6 alkoxy optionally substituted with C1-C6 alkoxy optionally substituted with —S(═O)₂NR^{C}R^{D}; or 5-6 membered heterocyclyl optionally substituted with C1-C6 alkyl, (viii) —C(═O)C6-C10 aryloxy;
(ix) —C(═O)-(5-6 membered heterocyclyloxy) optionally substituted with 1-4 substituents selected from hydroxyl and —C(═O)OH,
(x) —C(═O)NR^{A2}R^{B2},
(xi) C1-C6 haloalkyl optionally substituted with hydroxyl,
(xii) 5-6 membered heteroaryl optionally substituted with hydroxyl, and
(xiii) —C(═O)NHS(═O)₂R^{F};
R^{A1} and R^{B1} are independently selected from hydrogen and C1-C6 alkyl optionally substituted with hydroxyl;
R^{A2} and R^{B2} are independently selected from hydrogen and C1-C6 alkyl optionally substituted with hydroxyl or —S(═O)₂NH₂;
R^{C} and R^{D} are independently selected from hydrogen and C1-C6 alkyl; and
R^{F} is selected from:
(i) C1-C6 alkyl optionally substituted with 1-3 substituents selected from
(ii) hydroxyl,
(iii) halogen,
(iv) cyano,
(v) C1-C6 alkoxy,
(vi) 4-6-membered heterocyclyl optionally substituted with C1-C6 alkyl,
(vii) —C(═O)OH,
(viii) —OC(═O)C1-C6 alkyl,
(ix) —S(═O)₂C1-C6 alkyl,
(x) —C(═O)NR^{A1}R^{B1},
(xi) —NR^{C}R^{D},
(xii) —C(═O)C1-C6 aryloxy,
(xiii) C3-C6 cycloalkyl optionally substituted with —C(═O)OH, C1-C6 hydroxyalkyl, —C(═O)NR^{A1}R^{B1}, or —C(═O)C1-C6 alkoxy,
(xiv) —C(═O)C1-C6 alkoxy optionally substituted with —O(═O)C1-C6 alkyl, (xv) C2-C6 alkenyl,
(xvi) C1-C6 haloalkyl,
(xvii) phenyl,
(xviii) C3-C6 cycloalkyl optionally substituted with
   (a) C1-C6 alkyl optionally substituted with 1-3 substituents selected from hydroxyl, —C(═O)OH, —C(═O)NR^{A1}R^{B1}, or —C(═O)C1-C6 alkoxy,
   (b) cyano,
   (c) —C(═O)OH,
   (d) —C(═O)NR^{C}R^{D}, or
   (e)—C(═O)C1-C6 alkoxy optionally substituted with hydroxyl or —O(═O)C1-C6 alkyl, or
   (f) —NR^{C}R^{D}.

In some embodiments, R^{A} is hydrogen.

In some embodiments, R^{A} is C1-C6 alkyl. In some embodiments, R^{A} is methyl.

In some embodiments, R^{A} is C1-C6 alkoxy. In some embodiments, R^{A} is methoxy.

In some embodiments, Q is —C(═O)OH. In some embodiments, Q is —S(═O)₂OH. In some embodiments, Q is cyano. In some embodiments, Q is C1-C6 hydroxyalkyl. In some embodiments, Q is C1-C3 hydroxyalkyl. In some embodiments, Q is —C(═O)C6-C10 aryloxy. In some embodiments, Q is —S(O)₂NR^{A1}R^{B1}. In some embodiments, Q is —NR^{C}S(O)₂NR^{A1}R^{B1}. In some embodiments, Q is —C(═O)C1-C6 alkoxy optionally substituted with C1-C6 alkoxy optionally substituted with —S(═O)₂NR^{C}R^{D}. In some embodiments, Q is —C(═O)C1-C3 alkoxy optionally substituted with C1-C6 alkoxy. In some embodiments, Q is —C(═O)C1-C3 alkoxy. In some embodiments, Q is —C(═O)OEt. In some embodiments, Q is C(═O)C1-C6 alkoxy substituted with C1-C6 alkoxy optionally substituted with —S(═O)₂NR^{C}R^{D}. In some embodiments, Q is C(═O)C1-C6 alkoxy substituted with C1-C6 alkoxy substituted with —S(═O)₂NR^{C}R^{D}. In some embodiments, Q is C(═O)C1-C3 alkoxy substituted with C1-C3 alkoxy substituted with —S(═O)₂NR^{C}R^{D}. In some embodiments, Q is C(═O)C1-C6 alkoxy optionally substituted with 5-6 membered heterocyclyl optionally substituted with C1-C6 alkyl. In some embodiments, Q is C(═O)C1-C3 alkoxy substituted with 5-6 membered heterocyclyl optionally substituted with C1-C6 alkyl. In some embodiments, Q is C(═O)C1-C3 alkoxy substituted with 6 membered heterocyclyl optionally substituted with C1-C6 alkyl. In some embodiments, Q is C(═O)C1-C3 alkoxy substituted with 5 membered heterocyclyl optionally substituted with C1-C6 alkyl.

In some embodiments, Q is —C(═O)-(5-6 membered heterocyclyloxy) optionally substituted with 1-4 substituents selected from C1-C6 alkyl, hydroxyl, and —C(═O)OH. In some embodiments, Q is —C(═O)-(5 membered heterocyclyloxy) optionally substituted with 1-4 substituents selected from C1-C6 alkyl, hydroxyl, and —C(═O)OH. In some embodiments, Q is —C(═O)-(6 membered heterocyclyloxy) optionally substituted with 1-4 substituents selected from C1-C6 alkyl, hydroxyl, and —C(═O)OH. In some embodiments, Q is

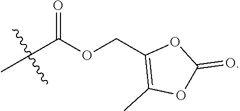 [SMILES: *C(=O)OCc1oc(=O)oc1C]

In some embodiments, Q is —C(═O)NR^{A2}R^{B2}. In some embodiments, each of R^{A2} and R^{B2} is hydrogen. In some embodiments, one of R^{A2} and R^{B2} is hydrogen and the other one of R^{A2} and R^{B2} is C1-C6 alkyl optionally substituted with hydroxyl or —S(═O)₂NH₂. In some embodiments, one of R^{A2} and R^{B2} is hydrogen and the other one of R^{A2} and R^{B2} is C1-C6 alkyl substituted with hydroxyl. In some embodiments, one of R^{A2} and R^{B2} is hydrogen and the other one of R^{A2} and R^{B2} is —CH₂CH₂OH. In some embodiments, one of R^{A2} and R^{B2} is hydrogen and the other one of R^{A2} and R^{B2} is C1-C6 alkyl substituted with —S(═O)₂NH₂. In some embodiments, one of R^{A2} and R^{B2} is hydrogen and the other one of R^{A2} and R^{B2} is

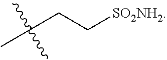 [SMILES: *CCS(N)(=O)=O]

In some embodiments, one of R^{A2} and R^{B2} is hydrogen and the other one of R^{A2} and R^{B2} is unsubstituted C1-C6 alkyl. In some embodiments, each of R^{A2} and R^{B2} is independently selected C1-C6 alkyl optionally substituted with hydroxyl or —S(═O)₂NH₂. In some embodiments, each of R^{A2} and R^{B2} is independently selected unsubstituted C1-C6 alkyl.

In some embodiments, Q is C1-C6 haloalkyl optionally substituted with hydroxyl.

In some embodiments, Q is C1-C6 haloalkyl substituted with hydroxyl.

In some embodiments, Q is unsubstituted C1-C6 haloalkyl.

In some embodiments, Q is 5-6 membered heteroaryl optionally substituted with hydroxyl.

In some embodiments, Q is 6 membered heteroaryl optionally substituted with hydroxyl.

In some embodiments, Q is selected from the group consisting of pyridinyl, pyrimidinyl, pyrazinyl, pyridazinyl, and triazinyl.

In some embodiments, Q is 5 membered heteroaryl optionally substituted with hydroxyl.

In some embodiments, Q is selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl.

In some embodiments, Q is

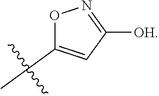 [SMILES: *c1cc(O)no1]

In some embodiments, Q is

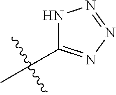 [SMILES: *c1nnn[nH]1]

In some embodiments, Q is unsubstituted 5-6 membered heteroaryl.

In some embodiments, Q is —C(═O)NHS(═O)₂R^{F}.

In some embodiments, R^{F} is C2-C6 alkenyl.

In some embodiments, R^{F} is

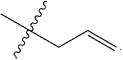 [SMILES: *CC=C]

In some embodiments, R^{F} is

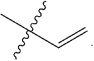 [SMILES: *C=C]

In some embodiments, R^{F} is C1-C6 haloalkyl.

In some embodiments, R^{F} is —CF₃ or CHF₂.

In some embodiments, R^{F} is phenyl.

In some embodiments, R^{F} is —NR^{C}R^{D}.

In some embodiments, R^{F} is C1-C6 alkyl optionally substituted with 1-3 substituents selected from hydroxyl; halogen; cyano; C1-C6 alkoxy; 4-6-membered heterocyclyl optionally substituted with C1-C6 alkyl; —C(═O)OH; —OC(═O)C1-C6 alkyl; —S(═O)₂C1-C6 alkyl; —C(═O)NR^{A1}R^{B1}; —NR^{C}R^{D}; —C(═O)C1-C6 aryloxy; C3-C6 cycloalkyl optionally substituted with —C(═O)OH, cyano, C1-C6 hydroxyalkyl, —C(═O)NR^{A1}R^{B1}, or —C(═O)C1-C6 alkoxy; or —C(═O)C1-C6 alkoxy optionally substituted with —C(═O)C1-C6 alkyl or —OC(═O)C1-C6 alkyl.

In some embodiments, R^{F} is C1-C6 alkyl substituted with 1-2 hydroxyl.

In some embodiments, R^{F} is selected from the group consisting of

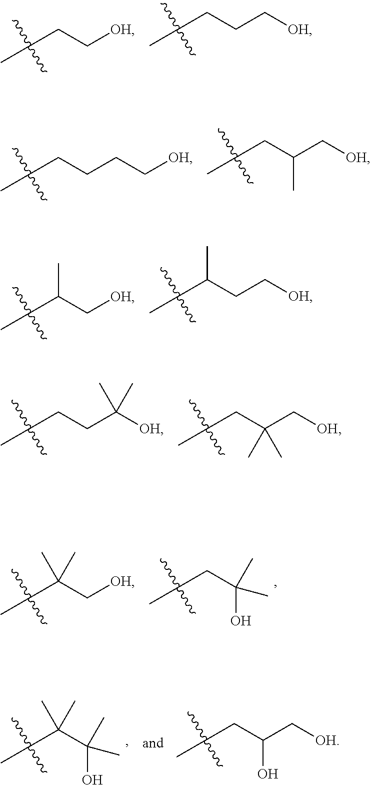

In some embodiments, R^{F} is C1-C6 alkyl substituted with cyano.

In some embodiments, R^{F} is

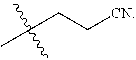 [SMILES: *CCC#N]

In some embodiments, R^{F} is C1-C6 alkyl substituted with C1-C6 alkoxy.

In some embodiments, R^{F} is selected from the group consisting of

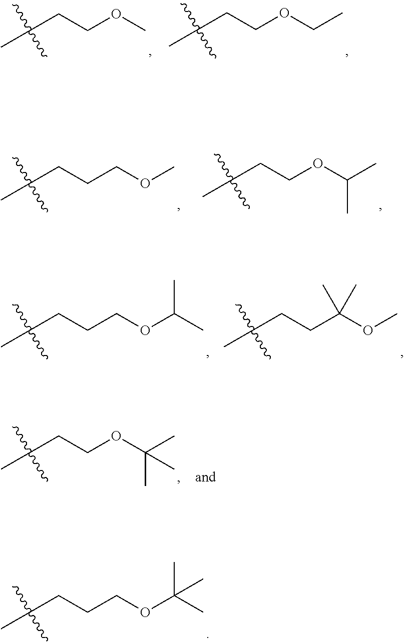

In some embodiments, R^{F} is C1-C6 alkyl substituted with 4-6-membered heterocyclyl optionally substituted with C1-C6 alkyl.

In some embodiments, R^{F} is

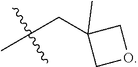 [SMILES: *CC1(C)COC1]

In some embodiments, R^{F} is C1-C6 alkyl substituted with —C(═O)OH.

In some embodiments, R^{F} is selected from the group consisting of

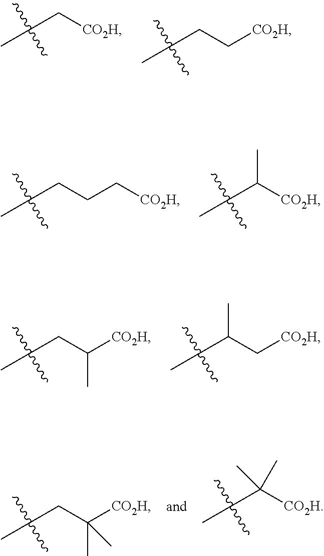

In some embodiments, R^{F} is C1-C6 alkyl substituted with —OC(═O)C1-C6 alkyl.

In some embodiments, R^{F} is

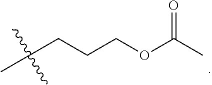 [SMILES: *CCCOC(C)=O]

In some embodiments, R^{F} is C1-C6 alkyl substituted with —S(═O)₂C1-C6 alkyl.

In some embodiments, R^{F} is

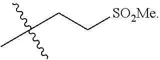 [SMILES: *CCS(C)(=O)=O]

In some embodiments, R^{F} is C1-C6 alkyl substituted with —C(═O)NR^{A1}R^{B1}.

In some embodiments, R^{F} is selected from the group consisting of

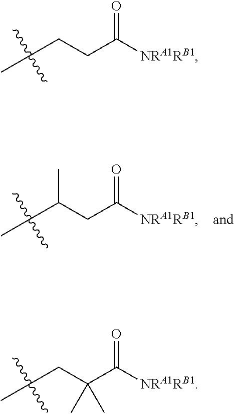

In some embodiments, R^{F} is

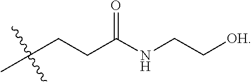 [SMILES: *CCC(=O)NCCO]

In some embodiments, R^{F} is C1-C6 alkyl substituted with 1-3 substituents selected from halogen, —C(═O)NR^{A1}R^{B1}, and —C(═O)OH.

In some embodiments, R^{F} is selected from the group consisting of

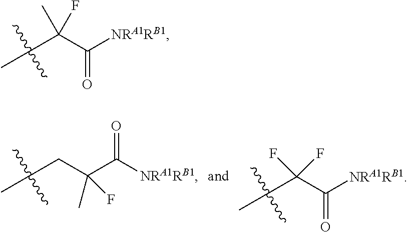

In some embodiments, R^{F} is

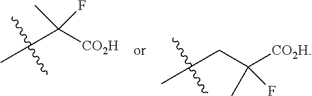

In some embodiments, R^{F} is C1-C6 alkyl substituted with NR^{C}R^{D}.

In some embodiments, R^{F} is C1-C6 alkyl substituted with —C(═O)C1-C6 aryloxy.

In some embodiments, R^{F} is C1-C6 alkyl substituted with C3-C6 cycloalkyl optionally substituted with —C(═O)OH, C1-C6 hydroxyalkyl, —C(═O)NR^{A1}R^{B1}, or —C(═O)C1-C6 alkoxy.

In some embodiments, R^{F} is C1-C6 alkyl substituted with C3-C4 cycloalkyl substituted with —C(═O)OH, C1-C6 hydroxyalkyl, —C(═O)NR^{A1}R^{B1}, or —C(═O)C1-C6 alkoxy.

In some embodiments, R^{F} is selected from the group consisting of

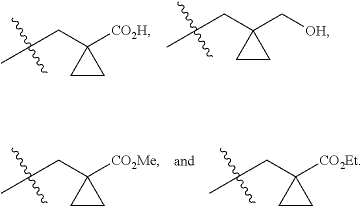 [SMILES: *CC1(C(=O)O)CC1 ; *CC1(CO)CC1 ; *CC1(C(=O)OC)CC1 ; *CC1(C(=O)OCC)CC1]

In some embodiments, R^{F} is

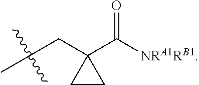

In some embodiments, R^{F} is C1-C6 alkyl substituted with —C(═O)C1-C6 alkoxy optionally substituted with —C(═O)C1-C6 alkyl.

In some embodiments, R^{F} is C1-C6 alkyl substituted with C(═O)C1-C4 alkoxy substituted with —C(═O)C1-C6 alkyl.

In some embodiments, R^{F} is C1-C6 alkyl substituted with C(═O)C1-C6 alkoxy.

In some embodiments, R^{F} is selected from the group consisting of

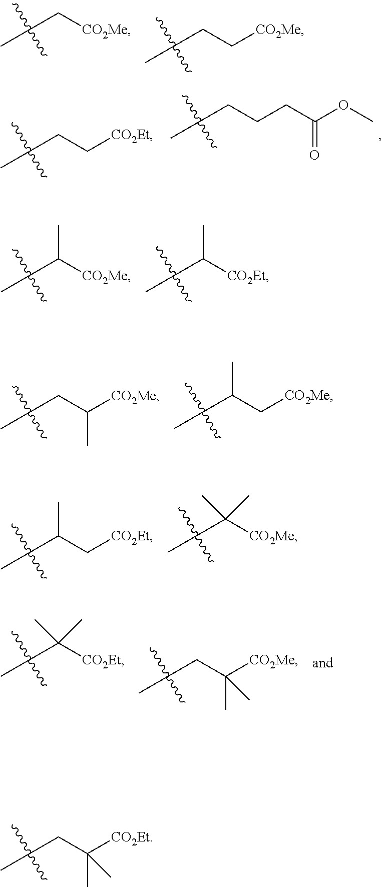

In some embodiments, R^{F} is C1-C6 alkyl optionally substituted with C(═O)OH. In some embodiments, R^{F} is C1-C6 alkyl substituted with —C(═O)OH.

In some embodiments, R^{F} is unsubstituted C1-C6 alkyl.

In some embodiments, R^{F} is selected from the group consisting of methyl, ethyl, n-propyl, isopropyl.

In some embodiments, R^{F} is C3-C6 cycloalkyl optionally substituted with C1-C6 alkyl optionally substituted with 1-3 substituents selected from hydroxyl, —C(═O)OH, —C(═O)NR^{A1}R^{B1}, or —C(═O)C1-C6 alkoxy; cyano; —C(═O)OH; —C(═O)NR^{C}R^{D}; or —C(═O)C1-C6 alkoxy optionally substituted with hydroxyl or —C(═O)C1-C6 alkyl.

In some embodiments, R^{F} is C3-C6 cycloalkyl substituted with C1-C6 alkyl substituted with 1-2 hydroxyl.

In some embodiments, R^{F} is C3-C4 cycloalkyl substituted with C1-C6 alkyl substituted with 1-2 hydroxyl.

In some embodiments, R^{F} is cyclopropyl substituted with C1-C6 alkyl substituted with 1-2 hydroxyl.

In some embodiments, R^{F} is selected from the group consisting of

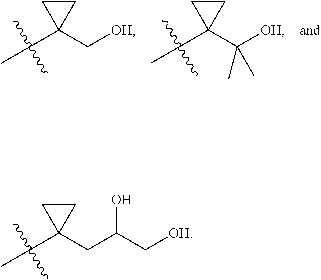

In some embodiments, R^{F} is C3-C6 cycloalkyl substituted with C1-C6 alkyl substituted with —C(═O)OH.

In some embodiments, R^{F} is C3-C4 cycloalkyl substituted with C1-C6 alkyl substituted with —C(═O)OH.

In some embodiments, R^{F} is cyclopropyl substituted with C1-C6 alkyl substituted with —C(═O)OH.

In some embodiments, R^{F} is

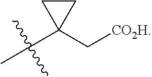 [SMILES: *C1(CC(=O)O)CC1]

In some embodiments, R^{F} is C3-C6 cycloalkyl substituted with C1-C6 alkyl substituted with —C(═O)NR^{A1}R^{B1}.

In some embodiments, R^{F} is C3-C4 cycloalkyl substituted with C1-C6 alkyl substituted with —C(═O)NR^{A1}R^{B1}.

In some embodiments, R^{F} is cyclopropyl substituted with C1-C6 alkyl substituted with —C(═O)NR^{A1}R^{B1}.

In some embodiments, R^{F} is

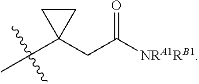

In some embodiments, R^{F} is C3-C6 cycloalkyl substituted with C1-C6 alkyl substituted with —C(═O)C1-C6 alkoxy.

In some embodiments, R^{F} is C3-C4 cycloalkyl substituted with C1-C6 alkyl substituted with —C(═O)C1-C6 alkoxy.

In some embodiments, R^{F} is cyclopropyl substituted with C1-C6 alkyl substituted with —C(═O)C1-C6 alkoxy.

In some embodiments, R^{F} is selected from the group consisting of

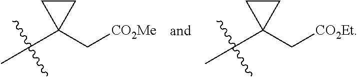 [SMILES: *C1(CC(=O)OC)CC1 ; *C1(CC(=O)OCC)CC1]

In some embodiments, R^{F} is C3-C6 cycloalkyl substituted with cyano.

In some embodiments, R^{F} is C3-C4 cycloalkyl substituted with cyano.

In some embodiments, R^{F} is

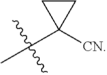 [SMILES: *C1(C#N)CC1]

In some embodiments, R^{F} is C3-C6 cycloalkyl substituted with —C(═O)OH.

In some embodiments, R^{F} is C3-C4 cycloalkyl substituted with —C(═O)OH.

In some embodiments, R^{F} is

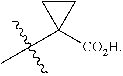 [SMILES: *C1(C(=O)O)CC1]

In some embodiments, R^{F} is C3-C6 cycloalkyl substituted with —C(═O)NR^{C}R^{D}.

In some embodiments, R^{F} is C3-C4 cycloalkyl substituted with —C(═O)NR^{C}R^{D}.

In some embodiments, R^{F} is

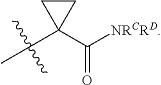

In some embodiments, R^{F} is C3-C6 cycloalkyl substituted with —C(═O)C1-C6 alkoxy substituted with hydroxyl.

In some embodiments, R^{F} is C3-C4 cycloalkyl substituted with —C(═O)C1-C6 alkoxy substituted with hydroxyl.

In some embodiments, R^{F} is

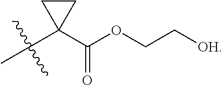 [SMILES: *C1(C(=O)OCCO)CC1]

In some embodiments, R^{F} is C3-C4 cycloalkyl substituted with —C(═O)C1-C6 alkoxy.

In some embodiments, R^{F} is selected from the group consisting of

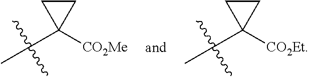 [SMILES: *C1(C(=O)OC)CC1 ; *C1(C(=O)OCC)CC1]

In some embodiments, R^{F} is C3-C6 cycloalkyl substituted with —C(═O)C1-C6 alkoxy substituted with —C(═O)C1-C6 alkyl.

In some embodiments, R^{F} is C3-C4 cycloalkyl substituted with —C(═O)C1-C6 alkoxy substituted with —C(═O)C1-C6 alkyl.

In some embodiments, R^{F} is C3-C6 cycloalkyl substituted with —C(═O)C1-C6 alkoxy substituted with —OC(═O)C1-C6 alkyl.

In some embodiments, R^{F} is C3-C4 cycloalkyl substituted with —C(═O)C1-C6 alkoxy substituted with —OC(═O)C1-C6 alkyl.

In some embodiments, R^{F} is

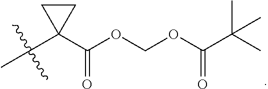 [SMILES: *C1(C(=O)OCOC(=O)C(C)(C)C)CC1]

In some embodiments, R^{F} is unsubstituted C3-C6 cycloalkyl.

In some embodiments, Q is selected from C(═O)OH; —S(═O)₂OH; 5-6 membered heteroaryl optionally substituted with hydroxyl; and-C(═O)NHS(═O)₂R^{F}. In some embodiments, Q is C(═O)OH or —C(═O)NHS(═O)₂R^{F}.

In some embodiments, each of R^{A1} and R^{B1} is hydrogen.

In some embodiments, one of R^{A1} and R^{B1} is hydrogen and the other one of R^{A1} and R^{B1} is C1-C6 alkyl optionally substituted with hydroxyl.

In some embodiments, one of R^{A1} and R^{B1} is hydrogen and the other one of R^{A1} and R^{B1} is C1-C3 alkyl.

In some embodiments, one of R^{A1} and R^{B1} is hydrogen and the other one of R^{A1} and R^{B1} is methyl.

In some embodiments, each of R^{A1} and R^{B1} is independently selected C1-C6 alkyl optionally substituted with hydroxyl.

In some embodiments, each of R^{A1} and R^{B1} is independently selected unsubstituted C1-C6 alkyl.

In some embodiments, R^{F} is —NR^{A3}R^{B3}.

In some embodiments, each of R^{A3} and R^{B3} is hydrogen.

In some embodiments, one of R^{A3} and R^{B3} is hydrogen and the other one of R^{A3} and R^{B3} is C1-C6 alkyl.

In some embodiments, one of R^{A3} and R^{B3} is hydrogen and the other one of R^{A3} and R^{B3} is C1-C3 alkyl.

In some embodiments, one of R^{A3} and R^{B3} is hydrogen and the other one of R^{A3} and R^{B3} is methyl.

In some embodiments, each of R^{A3} and R^{B3} is independently selected C1-C6 alkyl.

In some embodiments, each of R^{A3} and R^{B3} is independently selected unsubstituted C1-C6 alkyl.

In some embodiments, Ring B is 3-7 membered heterocyclylene.

In some embodiments, Ring B is selected from the group consisting of azetidinyl, oxetanyl, pyrrolidinyl, tetrahydrofuryl, thiolanyl, pyrazolinyl, oxathiolanyl, isoxazolidinyl, isothiazolidinyl, pyrrolinyl, pyrrolidinonyl, pyrazolidinyl, imidazolinyl, dioxolanyl, sulfolanyl, thiazolidedionyl, succinimidyl, dihydrofuranonyl, pyrazolidinonyl, oxazolidinyl, isoxazolidinonyl, hydantionyl, thiohydantionyl, imidazolidinonyl, oxazolidinonyl, thiazolidinonyl, oxathiolanonyl, dioxolanonyl, dioxazolidinonyl, oxadiazolidinonyl, triazolidinonyl, triazolidinethionyl, oxadiazolidinethionyl, dioxazolidinethionyl, dioxolanethionyl, oxazolidinethionyl, imidazolidinethionyl, isothiazolidinonyl, piperidinyl, tetrahydropyranyl, thianyl, morpholinyl, thiomorpholinyl, dioxanyl, piperazinyl, dithianyl, oxazinyl, tetrahydropyranonyl, piperidinonyl, dioxanonyl, oxazinanonyl, morpholinonyl, thiomorpholinonyl, piperazinonyl, tetrahydropyrimidinonyl, piperidinedionyl, oxazinanedionyl, dihydropyrimidindione, tetrahydropyridazinonyl, triazinanonyl, oxadiazinanonyl, dioxazinanonyl, morpholinedionyl, piperazinedionyl, piperazinetrionyl, triazinanedionyl, and azaspiro[3.3]heptanyl.

In some embodiments, Ring B is selected from the group consisting of azetidinyl, pyrrolidinyl, dihydropyrrolyl, piperidinyl, piperazinyl, and azaspiro[3.3]heptanyl.

In some embodiments, Ring B is selected from the group consisting of

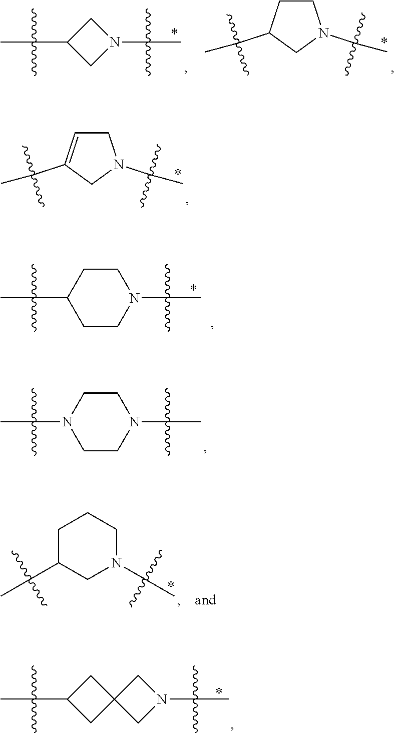

wherein “*” indicates the attachment point to Ring A.

In some embodiments, Ring B is

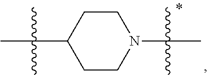 [SMILES: *C1CCN(*)CC1]

wherein “*” indicates the attachment point to Ring A.

In some embodiments, Ring B is 5-6 membered heteroarylene.

In some embodiments, Ring B is 5 membered heteroarylene selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl.

In some embodiments, Ring B is 6 membered heteroarylene selected from the group consisting of pyridinyl, pyrimidinyl, pyrazinyl, pyridazinyl, and triazinyl.

In some embodiments, Ring B is pyridyl.

In some embodiments, Ring B is

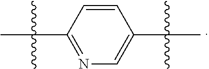 [SMILES: *c1ccc(*)nc1]

In some embodiments, Ring C is phenylene.

In some embodiments, Ring C is 5-6 membered heteroarylene.

In some embodiments, Ring C is 5 membered heteroarylene selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl.

In some embodiments, Ring C is selected from the group consisting of isoxazolyl, pyrazolyl, triazolyl, thiophenyl, and isothiazolyl.

In some embodiments, Ring C is selected from the group consisting of

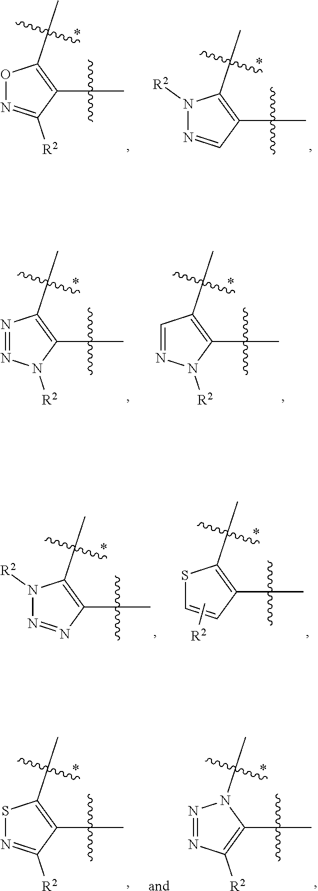

wherein “*” indicates the attachment point to Ring B.

In some embodiments, Ring C is

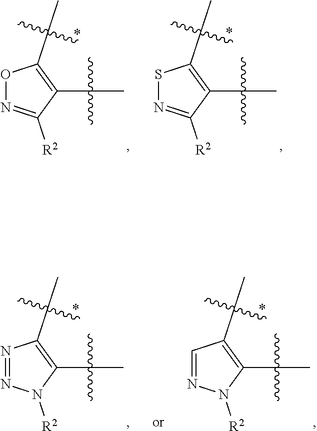

wherein “*” indicates the attachment point to Ring B.

In some embodiments, Ring C is

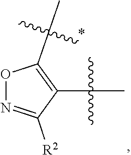

wherein “*” indicates the attachment point to Ring B.

In some embodiments, Ring C is 6 membered heteroarylene selected from the group consisting of pyridinyl, pyrimidinyl, pyrazinyl, pyridazinyl, and triazinyl.

In some embodiments, Ring C is pyrimidinyl.

In some embodiments, each R¹ is an independently selected C1-C6 alkyl.

In some embodiments, each R² is independently selected from C1-C6 alkyl, C1-C6 haloalkyl, C1-C6 alkoxy, and halogen.

In some embodiments, at least one R² is halogen.

In some embodiments, at least one R² is chloro.

In some embodiments, at least one R² is C1-C6 alkyl.

In some embodiments, at least one R² is methyl.

In some embodiments, at least one R² is C1-C6 haloalkyl.

In some embodiments, at least one R² is C1-C6 alkoxy.

In some embodiments, at least one R² is methoxy.

In some embodiments, X is —(CH₂)ₓNR^{G}C(═O)O—. In some embodiments, X is —CH₂NHC(═O)O—. In some embodiments, X is —NHC(═O)O—. In some embodiments, X is —N(CH₃)C(═O)O—.

In some embodiments, X is —(CH₂)ₓOC(═O)NR^{G}—. In some embodiments, X is —CH₂OC(═O)NH—. In some embodiments, X is —OC(═O)NH—.

In some embodiments, X is —(CH₂)ₓNR^{G}—. In some embodiments, X is —CH₂NH—. In some embodiments, X is —NH—.

In some embodiments, R^{G} is hydrogen.

In some embodiments, R^{G} is C1-C3 alkyl. In some embodiments, R^{G} is methyl.

In some embodiments, x is 0.

In some embodiments, x is 1.

In some embodiments, X is —CH(OH)—.

In some embodiments, R³ is hydrogen.

In some embodiments, R³ is C1-C6 alkyl optionally substituted with 1-2 R^{E}.

In some embodiments, at least one R^{E} is hydroxyl.

In some embodiments, at least one R^{E} is C1-C6 alkoxy.

In some embodiments, at least one R^{E} is phenyl optionally substituted with 1-3 substituents selected from C1-C6 alkyl, C1-C6 haloalkyl, C1-C6 alkoxyalkyl, C1-C6 alkoxy, hydroxyl, cyano, or halogen.

In some embodiments, at least one R^{E} is 5-10 membered heteroaryl optionally substituted with C1-C6 alkyl.

In some embodiments, at least one R^{E} is C3-C10 cycloalkyl.

In some embodiments, R³ is selected from the group consisting of

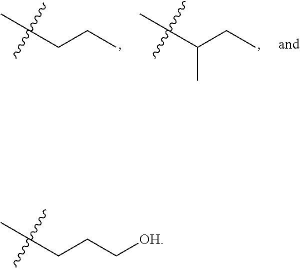

In some embodiments, R³ is

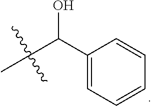 [SMILES: *C(O)c1ccccc1]

In some embodiments, R³ is

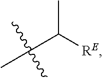

wherein R^{E} is phenyl optionally substituted with 1-3 substituents selected from C1-C3 alkyl, C1-C3 haloalkyl, C1-C3 alkoxyalkyl, C1-C63 alkoxy, hydroxyl, cyano, or halogen.

In some embodiments, R³ is

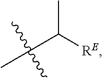

wherein R^{E} is phenyl substituted with 1-2 substituents selected from the group consisting of methyl, ethyl, isopropyl, chloro, fluoro, cyano, methoxy, and trifluoromethyl.

In some embodiments, R³ is

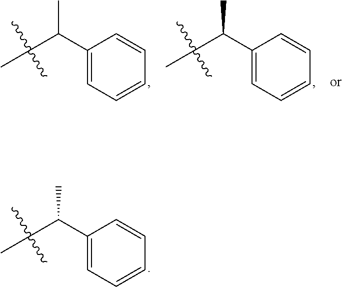

In some embodiments, R³ is

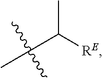

wherein R^{E} is 5-6 membered heteroaryl optionally substituted with C1-C6 alkyl.

In some embodiments, R³ is

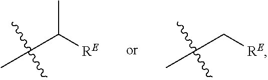

wherein R^{E} is cyclopropyl, cyclobutyl, cyclopentyl, or cyclohexyl.

In some embodiments, R³ is C6-C10 aryl optionally substituted with hydroxyl.

In some embodiments, R³ is phenyl substituted with hydroxyl.

In some embodiments, R³ is C5-C10 cycloalkyl.

In some embodiments, R³ is 5-6 membered heteroaryl optionally substituted with phenyl.

In some embodiments, R³ is 5 membered heteroaryl optionally substituted with phenyl.

In some embodiments, R³ is selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl, each optionally substituted with phenyl.

In some embodiments, R³ is pyrazolyl, oxazolyl, or oxadiazolyl, each optionally substituted with phenyl.

In some embodiments, R³ is selected from the group consisting of

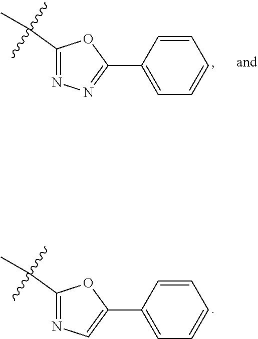 [SMILES: *c1nnc(-c2ccccc2)o1 ; *c1ncc(-c2ccccc2)o1]

In some embodiments, R³ is 6 membered heteroaryl optionally substituted with phenyl.

In some embodiments, R³ is selected from the group consisting of pyridinyl, pyrimidinyl, pyrazinyl, pyridazinyl, and triazinyl, each optionally substituted with phenyl.

In some embodiments, R³ is pyridyl optionally substituted with phenyl.

In some embodiments, R³ is

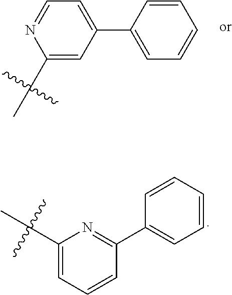 [SMILES: *c1cc(-c2ccccc2)ccn1 ; *c1cccc(-c2ccccc2)n1]

In some embodiments, R³ is unsubstituted 5-6 membered heteroaryl.

In some embodiments, R³ is 5-6 membered heterocyclyl optionally substituted with phenyl.

In some embodiments, R³ is 5 membered heterocyclyl optionally substituted with phenyl.

In some embodiments, R³ is pyrrolidine.

In some embodiments, R³ is 6 membered heterocyclyl optionally substituted with phenyl.

In some embodiments, R³ is dihydro-4H-1,3-oxazinyl.

In some embodiments, R³ is unsubstituted 5-6 membered heterocyclyl.

In some embodiments, R^{C} is hydrogen.

In some embodiments, R^{C} is C1-C6 alkyl.

In some embodiments, each of R^{C} and R^{D} is hydrogen.

In some embodiments, one of R^{C} and R^{D} is hydrogen and the other one of R^{C} and R^{D} is C1-C6 alkyl.

In some embodiments, each of R^{C} and R^{D} is independently selected C1-C6 alkyl.

In some embodiments, R^{D} is C1-C3 alkyl.

In some embodiments, R^{D} is methyl.

In some embodiments, m is 0.

In some embodiments, m is 1.

In some embodiments, n is 0.

In some embodiments, n is 1.

In some embodiments, n is 2.

In some embodiments, n is 3.

In some embodiments, Formula (I) is Formula (I-a):

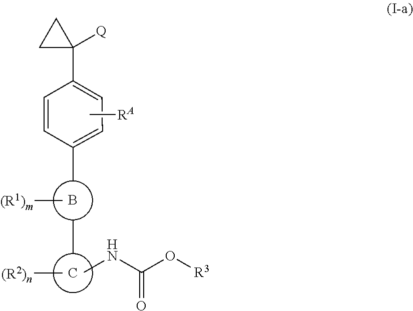

or pharmaceutically acceptable salts thereof.

In some embodiments, Formula (I) is Formula (I-b):

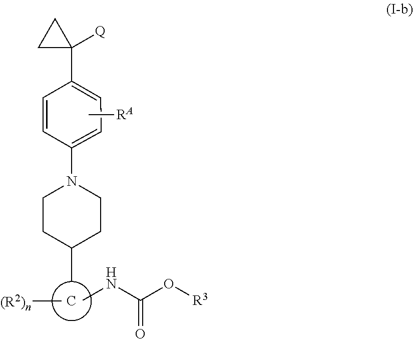

or pharmaceutically acceptable salts thereof.

In some embodiments, Formula (I) is Formula (I-c):

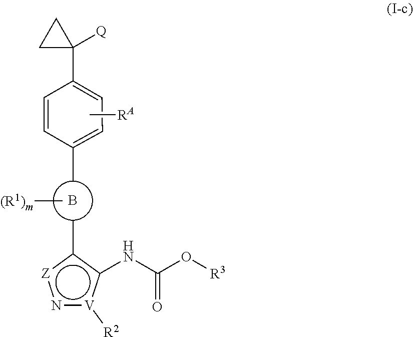

or pharmaceutically acceptable salts thereof, wherein Z is CH, N, O, or S; V is C or N; and wherein Z is 0 or S when V is C.

In some embodiments, Formula (I) is Formula (I-d):

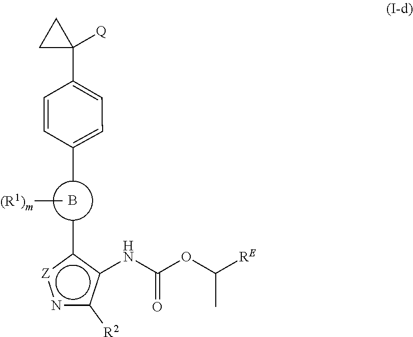

or pharmaceutically acceptable salts thereof, wherein Q is —CO₂H or —C(═O)NHS(═O)₂R^{F}; and Z is O or S.

In some embodiments, Formula (I) is Formula (I-e):

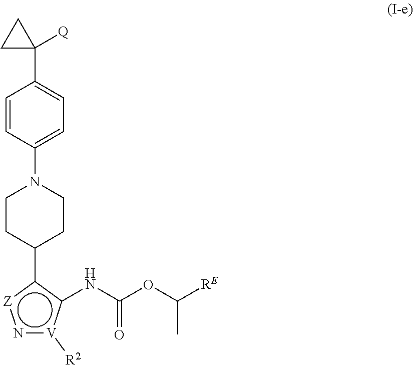

or pharmaceutically acceptable salts thereof,
wherein Q is —CO₂H or —C(═O)NHS(═O)₂R^{F}; Z is CH, N, O, or S; V is C or N; and wherein Z is 0 or S when V is C.

In some embodiments, Formula (I) is Formula (I-f):

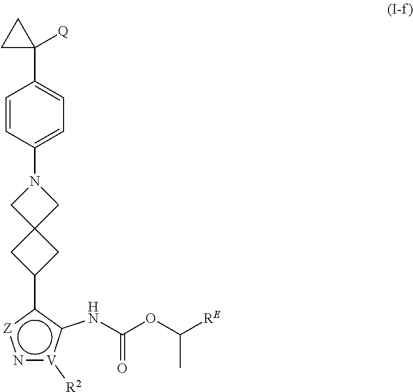

or pharmaceutically acceptable salts thereof,
wherein Q is —CO₂H or —C(═O)NHS(═O)₂R^{F}; Z is CH, N, O, or S; V is C or N; and wherein Z is 0 or S when V is C.

### Non-Limiting Exemplary Compounds

In some embodiments, the compound is selected from the group consisting of the compounds in Examples 1-31, or a pharmaceutically acceptable salt thereof.

In some embodiments, the compound is selected from the group consisting of the compounds delineated in Table A, or a pharmaceutically acceptable salt thereof.

TABLE A

| Cmpd # | Structure |
|---|---|
| 1 | 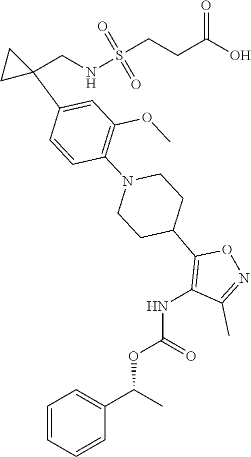 [SMILES: COc1cc(C2(CNS(=O)(=O)CCC(=O)O)CC2)ccc1N1CCC(c2onc(C)c2NC(=O)O[C@H](C)c2ccccc2)CC1] |
| 2 | 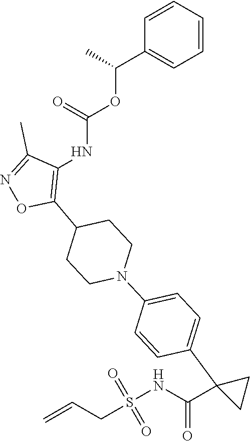 [SMILES: C=CCS(=O)(=O)NC(=O)C1(c2ccc(N3CCC(c4onc(C)c4NC(=O)O[C@H](C)c4ccccc4)CC3)cc2)CC1] |
| 3 | 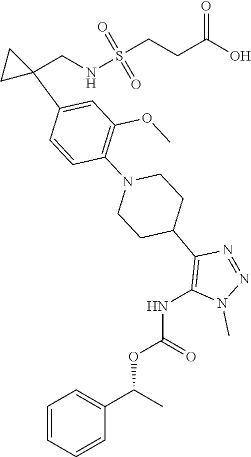 [SMILES: COc1cc(C2(CNS(=O)(=O)CCC(=O)O)CC2)ccc1N1CCC(c2nnn(C)c2NC(=O)O[C@H](C)c2ccccc2)CC1] |
| 4 | 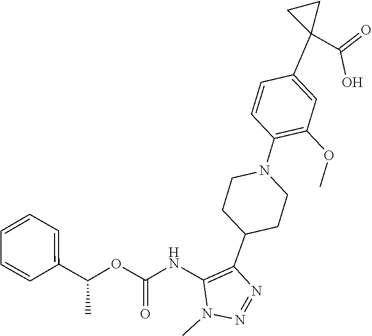 [SMILES: COc1cc(C2(C(=O)O)CC2)ccc1N1CCC(c2nnn(C)c2NC(=O)O[C@H](C)c2ccccc2)CC1] |
| 5 | 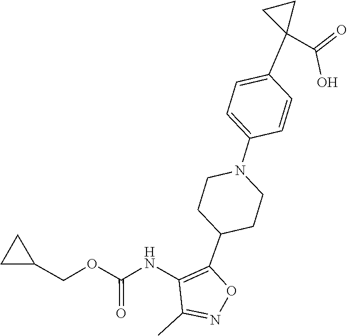 [SMILES: Cc1noc(C2CCN(c3ccc(C4(C(=O)O)CC4)cc3)CC2)c1NC(=O)OCC1CC1] |
| 6 | 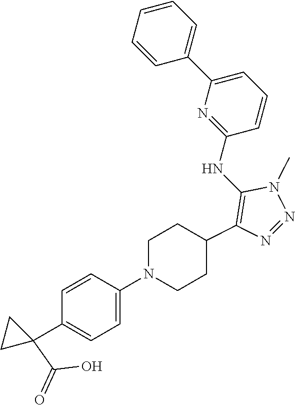 [SMILES: Cn1nnc(C2CCN(c3ccc(C4(C(=O)O)CC4)cc3)CC2)c1Nc1cccc(-c2ccccc2)n1] |
| 7 | 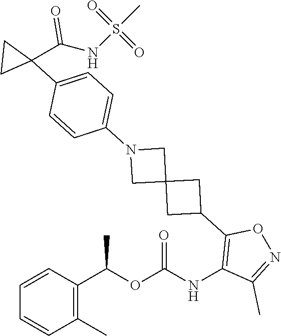 [SMILES: Cc1ccccc1[C@@H](C)OC(=O)Nc1c(C)noc1C1CC2(C1)CN(c1ccc(C3(C(=O)NS(C)(=O)=O)CC3)cc1)C2] |
| 8 | 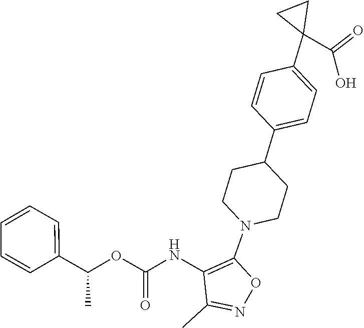 [SMILES: Cc1noc(N2CCC(c3ccc(C4(C(=O)O)CC4)cc3)CC2)c1NC(=O)O[C@H](C)c1ccccc1] |
| 9 | 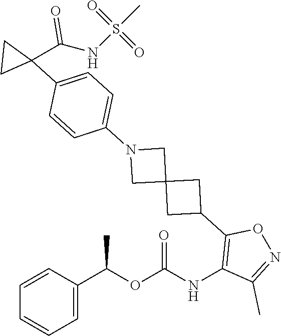 [SMILES: Cc1noc(C2CC3(C2)CN(c2ccc(C4(C(=O)NS(C)(=O)=O)CC4)cc2)C3)c1NC(=O)O[C@H](C)c1ccccc1] |
| 10 | 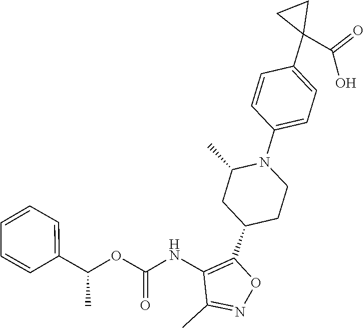 [SMILES: Cc1noc([C@H]2CCN(c3ccc(C4(C(=O)O)CC4)cc3)[C@@H](C)C2)c1NC(=O)O[C@H](C)c1ccccc1] |
| 11 | 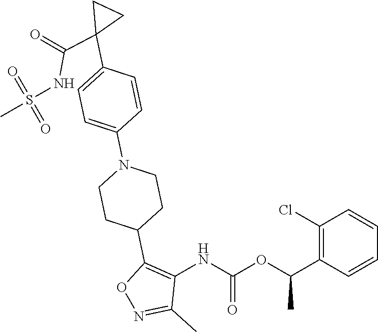 [SMILES: Cc1noc(C2CCN(c3ccc(C4(C(=O)NS(C)(=O)=O)CC4)cc3)CC2)c1NC(=O)O[C@H](C)c1ccccc1Cl] |
| 12 | 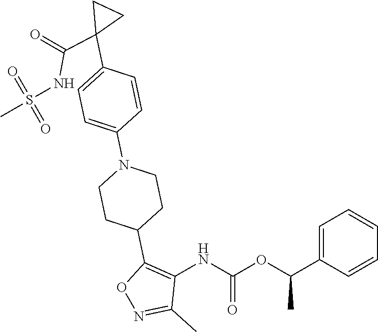 [SMILES: Cc1noc(C2CCN(c3ccc(C4(C(=O)NS(C)(=O)=O)CC4)cc3)CC2)c1NC(=O)O[C@H](C)c1ccccc1] |
| 13 | 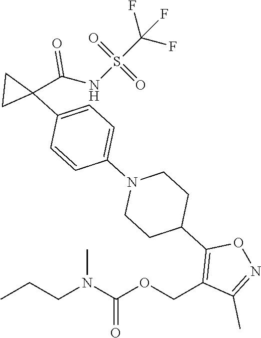 [SMILES: CCCN(C)C(=O)OCc1c(C)noc1C1CCN(c2ccc(C3(C(=O)NS(=O)(=O)C(F)(F)F)CC3)cc2)CC1] |
| 14 | 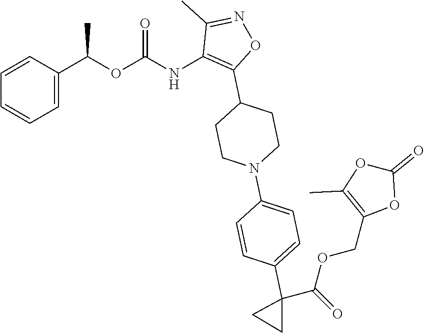 [SMILES: Cc1noc(C2CCN(c3ccc(C4(C(=O)OCc5oc(=O)oc5C)CC4)cc3)CC2)c1NC(=O)O[C@H](C)c1ccccc1] |
| 15 | 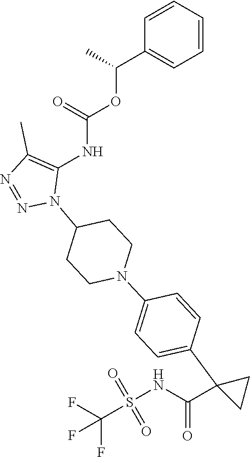 [SMILES: Cc1nnn(C2CCN(c3ccc(C4(C(=O)NS(=O)(=O)C(F)(F)F)CC4)cc3)CC2)c1NC(=O)O[C@H](C)c1ccccc1] |
| 16 | 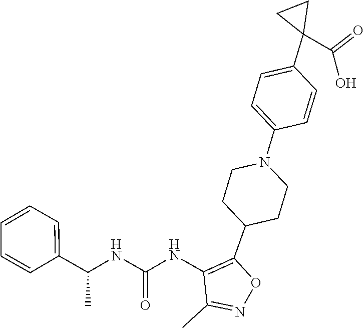 [SMILES: Cc1noc(C2CCN(c3ccc(C4(C(=O)O)CC4)cc3)CC2)c1NC(=O)N[C@H](C)c1ccccc1] |
| 17 | 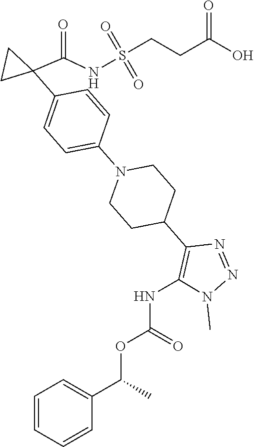 [SMILES: C[C@@H](OC(=O)Nc1c(C2CCN(c3ccc(C4(C(=O)NS(=O)(=O)CCC(=O)O)CC4)cc3)CC2)nnn1C)c1ccccc1] |
| 18 | 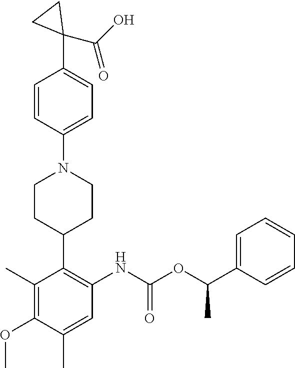 [SMILES: COc1c(C)cc(NC(=O)O[C@H](C)c2ccccc2)c(C2CCN(c3ccc(C4(C(=O)O)CC4)cc3)CC2)c1C] |
| 19 | 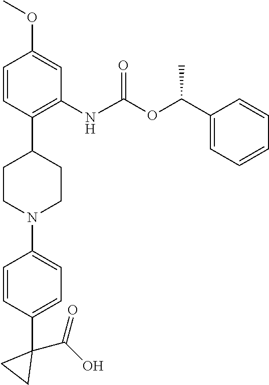 [SMILES: COc1ccc(C2CCN(c3ccc(C4(C(=O)O)CC4)cc3)CC2)c(NC(=O)O[C@H](C)c2ccccc2)c1] |
| 20 | 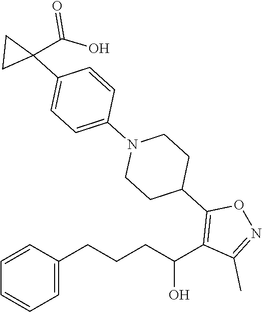 [SMILES: Cc1noc(C2CCN(c3ccc(C4(C(=O)O)CC4)cc3)CC2)c1C(O)CCCc1ccccc1] |
| 21 | 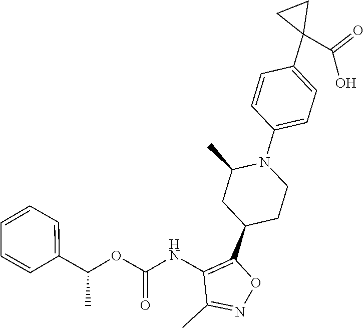 [SMILES: Cc1noc([C@@H]2CCN(c3ccc(C4(C(=O)O)CC4)cc3)[C@H](C)C2)c1NC(=O)O[C@H](C)c1ccccc1] |
| 22 | 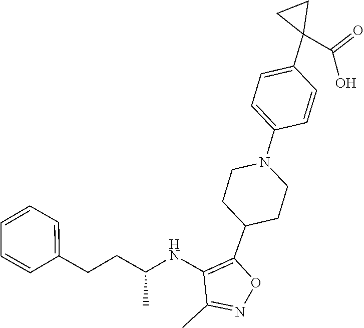 [SMILES: Cc1noc(C2CCN(c3ccc(C4(C(=O)O)CC4)cc3)CC2)c1N[C@H](C)CCc1ccccc1] |
| 23 | 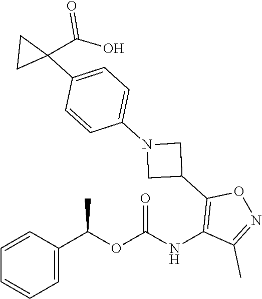 [SMILES: Cc1noc(C2CN(c3ccc(C4(C(=O)O)CC4)cc3)C2)c1NC(=O)O[C@H](C)c1ccccc1] |
| 24 | 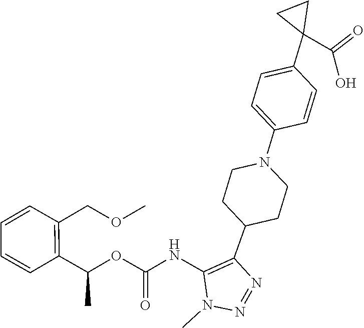 [SMILES: COCc1ccccc1[C@H](C)OC(=O)Nc1c(C2CCN(c3ccc(C4(C(=O)O)CC4)cc3)CC2)nnn1C] |
| 25 | 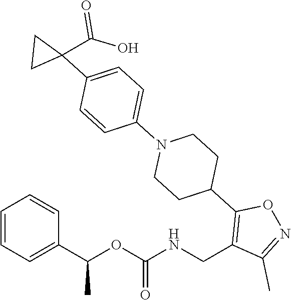 [SMILES: Cc1noc(C2CCN(c3ccc(C4(C(=O)O)CC4)cc3)CC2)c1CNC(=O)O[C@@H](C)c1ccccc1] |
| 26 | 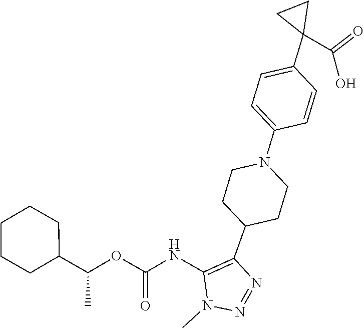 [SMILES: C[C@@H](OC(=O)Nc1c(C2CCN(c3ccc(C4(C(=O)O)CC4)cc3)CC2)nnn1C)C1CCCCC1] |
| 27 | 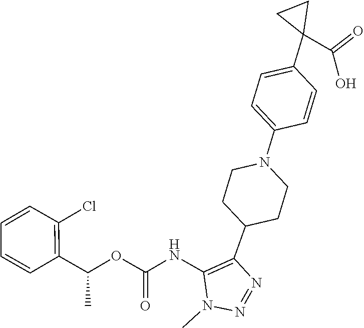 [SMILES: C[C@@H](OC(=O)Nc1c(C2CCN(c3ccc(C4(C(=O)O)CC4)cc3)CC2)nnn1C)c1ccccc1Cl] |
| 28 | 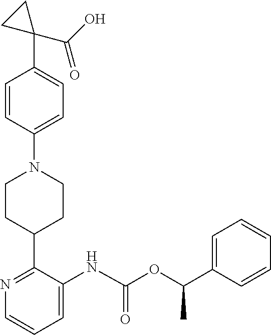 [SMILES: C[C@@H](OC(=O)Nc1cccnc1C1CCN(c2ccc(C3(C(=O)O)CC3)cc2)CC1)c1ccccc1] |
| 29 | 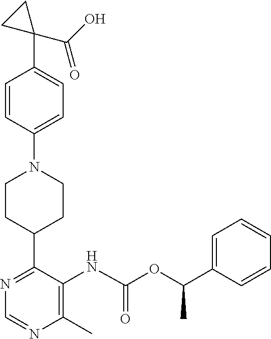 [SMILES: Cc1ncnc(C2CCN(c3ccc(C4(C(=O)O)CC4)cc3)CC2)c1NC(=O)O[C@H](C)c1ccccc1] |
| 30 | 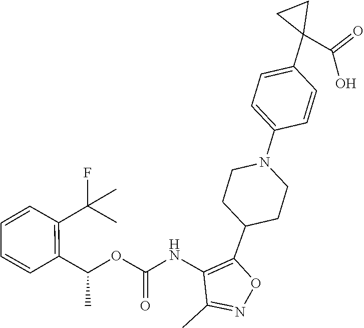 [SMILES: Cc1noc(C2CCN(c3ccc(C4(C(=O)O)CC4)cc3)CC2)c1NC(=O)O[C@H](C)c1ccccc1C(C)(C)F] F |
| 31 | 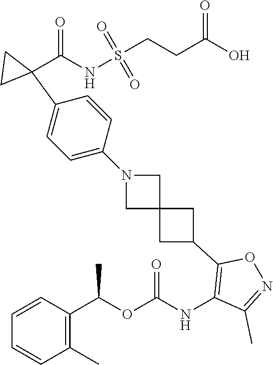 [SMILES: Cc1ccccc1[C@@H](C)OC(=O)Nc1c(C)noc1C1CC2(C1)CN(c1ccc(C3(C(=O)NS(=O)(=O)CCC(=O)O)CC3)cc1)C2] |
| 32 | 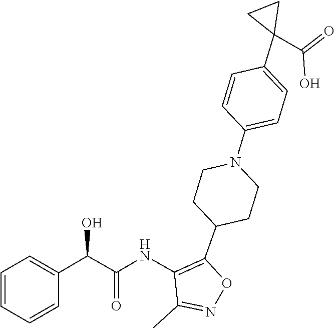 [SMILES: Cc1noc(C2CCN(c3ccc(C4(C(=O)O)CC4)cc3)CC2)c1NC(=O)[C@H](O)c1ccccc1] |
| 33 | 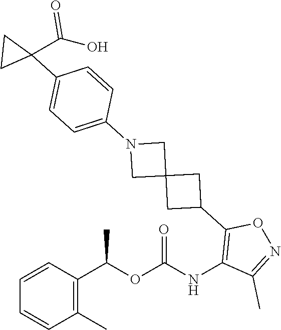 [SMILES: Cc1ccccc1[C@@H](C)OC(=O)Nc1c(C)noc1C1CC2(C1)CN(c1ccc(C3(C(=O)O)CC3)cc1)C2] |
| 34 | 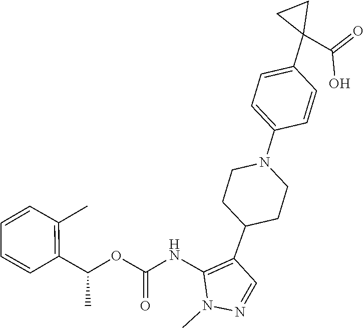 [SMILES: Cc1ccccc1[C@@H](C)OC(=O)Nc1c(C2CCN(c3ccc(C4(C(=O)O)CC4)cc3)CC2)cnn1C] |
| 35 | 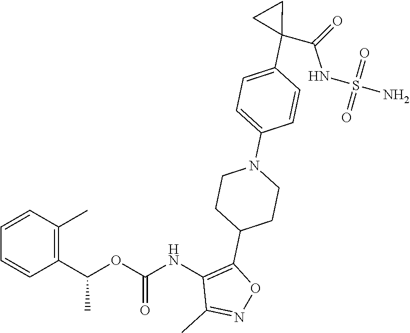 [SMILES: Cc1ccccc1[C@@H](C)OC(=O)Nc1c(C)noc1C1CCN(c2ccc(C3(C(=O)NS(N)(=O)=O)CC3)cc2)CC1] |
| 36 | 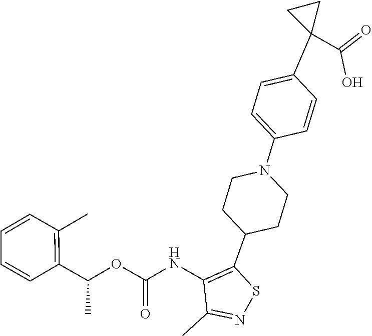 [SMILES: Cc1ccccc1[C@@H](C)OC(=O)Nc1c(C)nsc1C1CCN(c2ccc(C3(C(=O)O)CC3)cc2)CC1] |
| 37 | 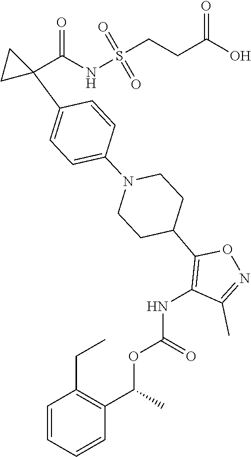 [SMILES: CCc1ccccc1[C@@H](C)OC(=O)Nc1c(C)noc1C1CCN(c2ccc(C3(C(=O)NS(=O)(=O)CCC(=O)O)CC3)cc2)CC1] |
| 38 | 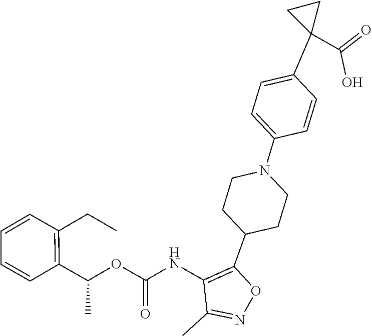 [SMILES: CCc1ccccc1[C@@H](C)OC(=O)Nc1c(C)noc1C1CCN(c2ccc(C3(C(=O)O)CC3)cc2)CC1] |
| 39 | 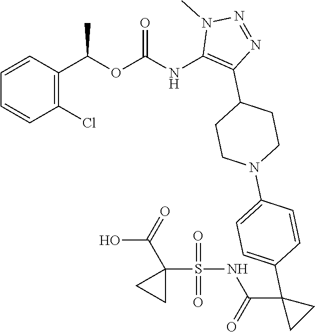 [SMILES: C[C@@H](OC(=O)Nc1c(C2CCN(c3ccc(C4(C(=O)NS(=O)(=O)C5(C(=O)O)CC5)CC4)cc3)CC2)nnn1C)c1ccccc1Cl] |
| 40 | 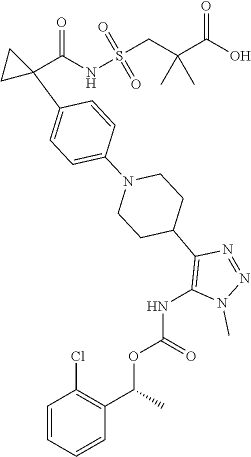 [SMILES: C[C@@H](OC(=O)Nc1c(C2CCN(c3ccc(C4(C(=O)NS(=O)(=O)CC(C)(C)C(=O)O)CC4)cc3)CC2)nnn1C)c1ccccc1Cl] |
| 41 | 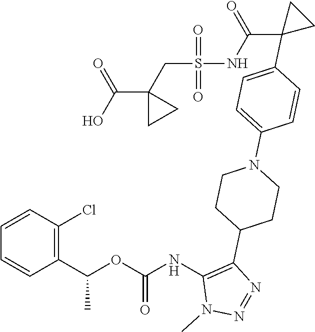 [SMILES: C[C@@H](OC(=O)Nc1c(C2CCN(c3ccc(C4(C(=O)NS(=O)(=O)CC5(C(=O)O)CC5)CC4)cc3)CC2)nnn1C)c1ccccc1Cl] |
| 42 | 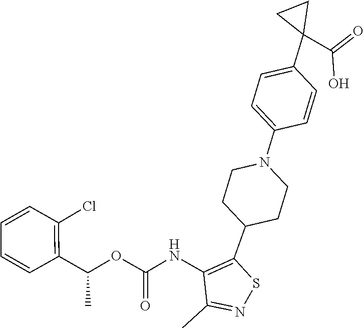 [SMILES: Cc1nsc(C2CCN(c3ccc(C4(C(=O)O)CC4)cc3)CC2)c1NC(=O)O[C@H](C)c1ccccc1Cl] |
| 43 | 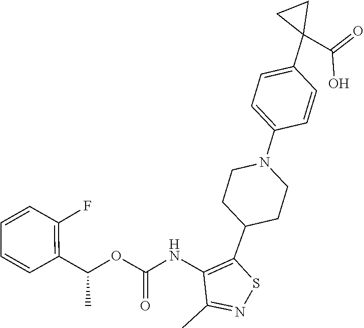 [SMILES: Cc1nsc(C2CCN(c3ccc(C4(C(=O)O)CC4)cc3)CC2)c1NC(=O)O[C@H](C)c1ccccc1F] |
| 44 | 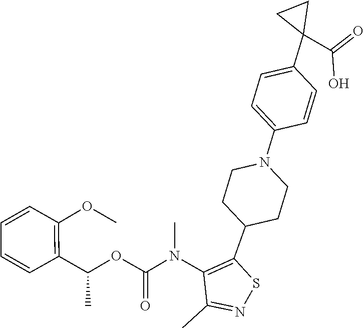 [SMILES: COc1ccccc1[C@@H](C)OC(=O)N(C)c1c(C)nsc1C1CCN(c2ccc(C3(C(=O)O)CC3)cc2)CC1] |
| 45 | 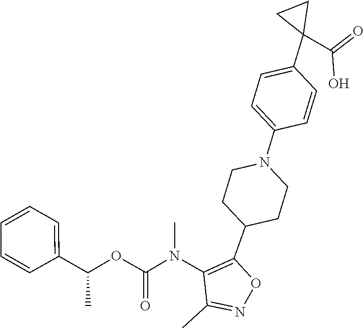 [SMILES: Cc1noc(C2CCN(c3ccc(C4(C(=O)O)CC4)cc3)CC2)c1N(C)C(=O)O[C@H](C)c1ccccc1] |
| 46 | 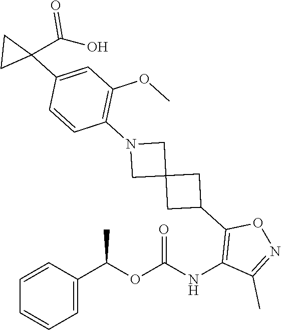 [SMILES: COc1cc(C2(C(=O)O)CC2)ccc1N1CC2(CC(c3onc(C)c3NC(=O)O[C@H](C)c3ccccc3)C2)C1] |
| 47 | 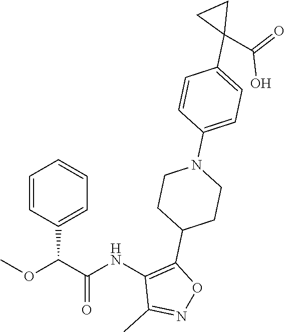 [SMILES: CO[C@@H](C(=O)Nc1c(C)noc1C1CCN(c2ccc(C3(C(=O)O)CC3)cc2)CC1)c1ccccc1] |
| 48 | 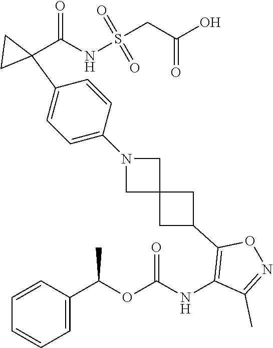 [SMILES: Cc1noc(C2CC3(C2)CN(c2ccc(C4(C(=O)NS(=O)(=O)CC(=O)O)CC4)cc2)C3)c1NC(=O)O[C@H](C)c1ccccc1] |
| 49 | 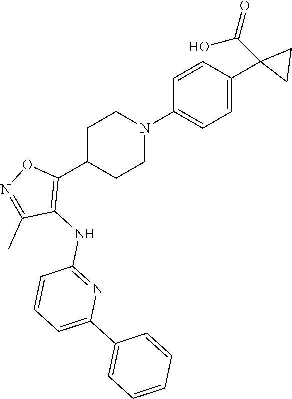 [SMILES: Cc1noc(C2CCN(c3ccc(C4(C(=O)O)CC4)cc3)CC2)c1Nc1cccc(-c2ccccc2)n1] |
| 50 | 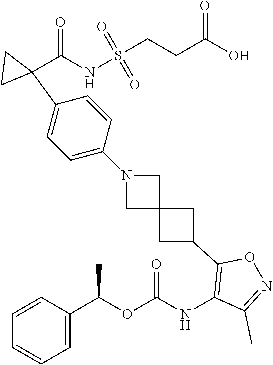 [SMILES: Cc1noc(C2CC3(C2)CN(c2ccc(C4(C(=O)NS(=O)(=O)CCC(=O)O)CC4)cc2)C3)c1NC(=O)O[C@H](C)c1ccccc1] |
| 51 | 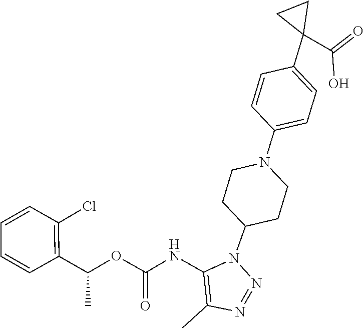 [SMILES: Cc1nnn(C2CCN(c3ccc(C4(C(=O)O)CC4)cc3)CC2)c1NC(=O)O[C@H](C)c1ccccc1Cl] |
| 52 | 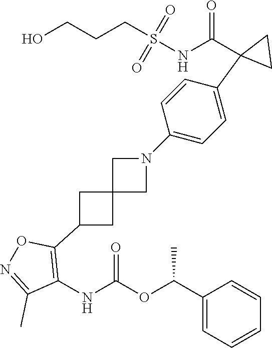 [SMILES: Cc1noc(C2CC3(C2)CN(c2ccc(C4(C(=O)NS(=O)(=O)CCCO)CC4)cc2)C3)c1NC(=O)O[C@H](C)c1ccccc1] |
| 53 | 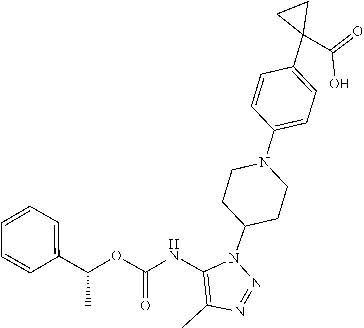 [SMILES: Cc1nnn(C2CCN(c3ccc(C4(C(=O)O)CC4)cc3)CC2)c1NC(=O)O[C@H](C)c1ccccc1] |
| 54 | 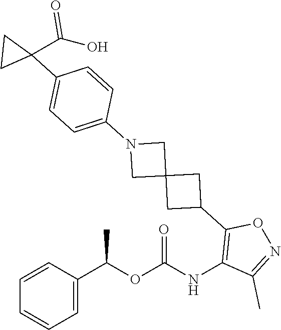 [SMILES: Cc1noc(C2CC3(C2)CN(c2ccc(C4(C(=O)O)CC4)cc2)C3)c1NC(=O)O[C@H](C)c1ccccc1] |
| 55 | 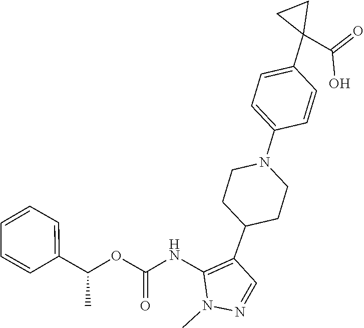 [SMILES: C[C@@H](OC(=O)Nc1c(C2CCN(c3ccc(C4(C(=O)O)CC4)cc3)CC2)cnn1C)c1ccccc1] |
| 56 | 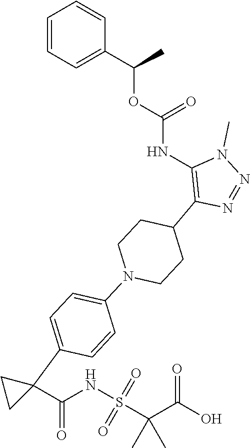 [SMILES: C[C@@H](OC(=O)Nc1c(C2CCN(c3ccc(C4(C(=O)NS(=O)(=O)C(C)(C)C(=O)O)CC4)cc3)CC2)nnn1C)c1ccccc1] |
| 57 | 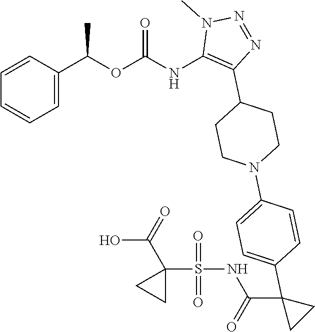 [SMILES: C[C@@H](OC(=O)Nc1c(C2CCN(c3ccc(C4(C(=O)NS(=O)(=O)C5(C(=O)O)CC5)CC4)cc3)CC2)nnn1C)c1ccccc1] |
| 58 | 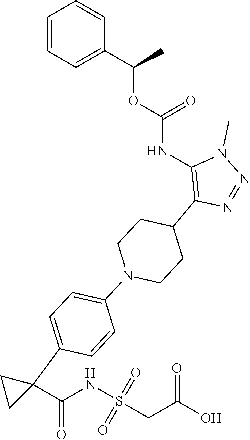 [SMILES: C[C@@H](OC(=O)Nc1c(C2CCN(c3ccc(C4(C(=O)NS(=O)(=O)CC(=O)O)CC4)cc3)CC2)nnn1C)c1ccccc1] |
| 59 | 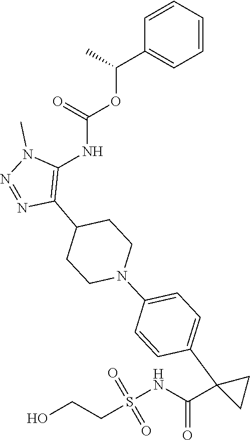 [SMILES: C[C@@H](OC(=O)Nc1c(C2CCN(c3ccc(C4(C(=O)NS(=O)(=O)CCO)CC4)cc3)CC2)nnn1C)c1ccccc1] |
| 60 | 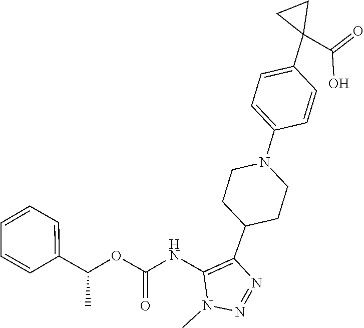 [SMILES: C[C@@H](OC(=O)Nc1c(C2CCN(c3ccc(C4(C(=O)O)CC4)cc3)CC2)nnn1C)c1ccccc1] |
| 61 | 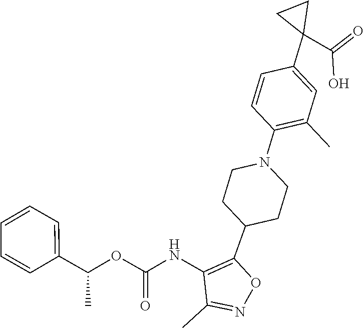 [SMILES: Cc1cc(C2(C(=O)O)CC2)ccc1N1CCC(c2onc(C)c2NC(=O)O[C@H](C)c2ccccc2)CC1] |
| 62 | 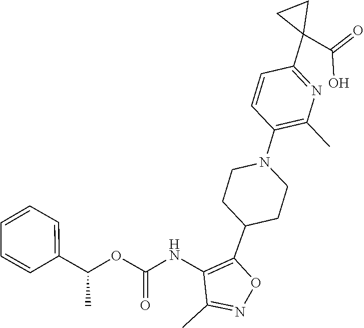 [SMILES: Cc1nc(C2(C(=O)O)CC2)ccc1N1CCC(c2onc(C)c2NC(=O)O[C@H](C)c2ccccc2)CC1] |
| 63 | 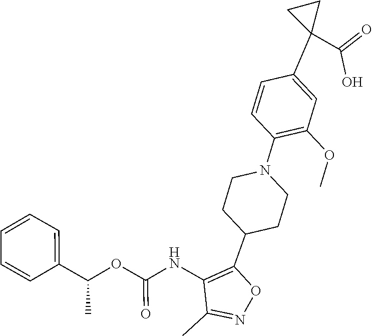 [SMILES: COc1cc(C2(C(=O)O)CC2)ccc1N1CCC(c2onc(C)c2NC(=O)O[C@H](C)c2ccccc2)CC1] |
| 64 | 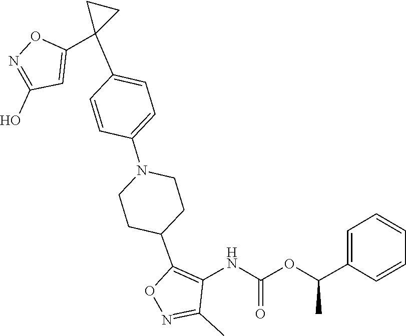 [SMILES: Cc1noc(C2CCN(c3ccc(C4(c5cc(O)no5)CC4)cc3)CC2)c1NC(=O)O[C@H](C)c1ccccc1] |
| 65 | 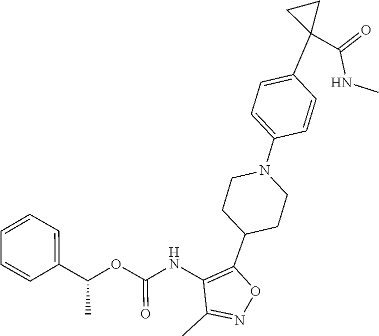 [SMILES: CNC(=O)C1(c2ccc(N3CCC(c4onc(C)c4NC(=O)O[C@H](C)c4ccccc4)CC3)cc2)CC1] |
| 66 | 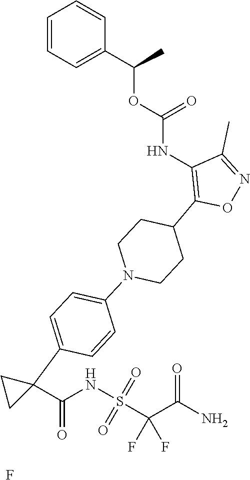 |
| 67 | 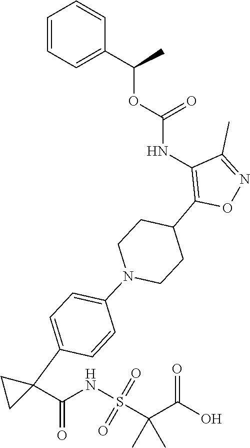 [SMILES: Cc1noc(C2CCN(c3ccc(C4(C(=O)NS(=O)(=O)C(C)(C)C(=O)O)CC4)cc3)CC2)c1NC(=O)O[C@H](C)c1ccccc1] |
| 68 | 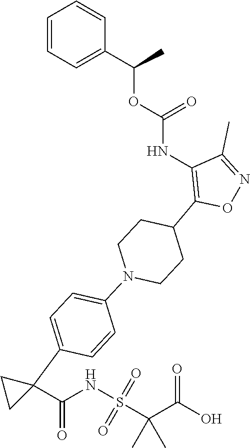 [SMILES: Cc1noc(C2CCN(c3ccc(C4(C(=O)NS(=O)(=O)C(C)(C)C(=O)O)CC4)cc3)CC2)c1NC(=O)O[C@H](C)c1ccccc1] |
| 69 | 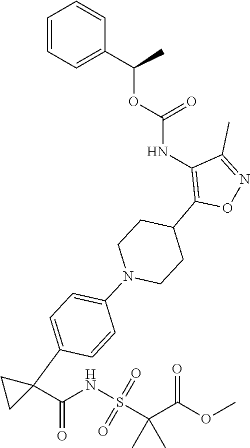 [SMILES: COC(=O)C(C)(C)S(=O)(=O)NC(=O)C1(c2ccc(N3CCC(c4onc(C)c4NC(=O)O[C@H](C)c4ccccc4)CC3)cc2)CC1] |
| 70 | 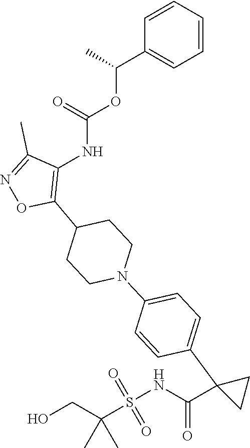 [SMILES: Cc1noc(C2CCN(c3ccc(C4(C(=O)NS(=O)(=O)C(C)(C)CO)CC4)cc3)CC2)c1NC(=O)O[C@H](C)c1ccccc1] |
| 71 | 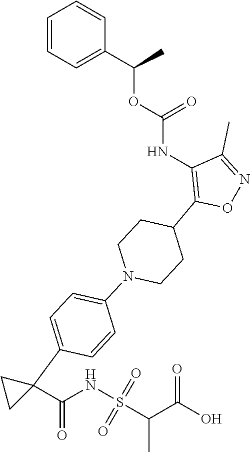 [SMILES: Cc1noc(C2CCN(c3ccc(C4(C(=O)NS(=O)(=O)C(C)C(=O)O)CC4)cc3)CC2)c1NC(=O)O[C@H](C)c1ccccc1] |
| 72 | 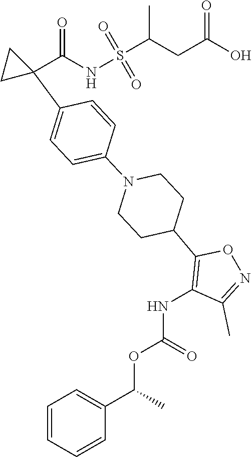 [SMILES: Cc1noc(C2CCN(c3ccc(C4(C(=O)NS(=O)(=O)C(C)CC(=O)O)CC4)cc3)CC2)c1NC(=O)O[C@H](C)c1ccccc1] |
| 73 | 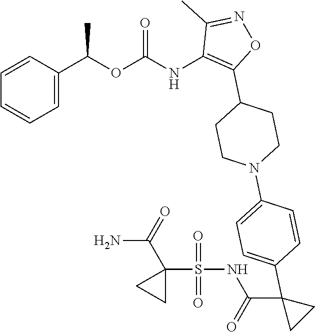 [SMILES: Cc1noc(C2CCN(c3ccc(C4(C(=O)NS(=O)(=O)C5(C(N)=O)CC5)CC4)cc3)CC2)c1NC(=O)O[C@H](C)c1ccccc1] |
| 74 | 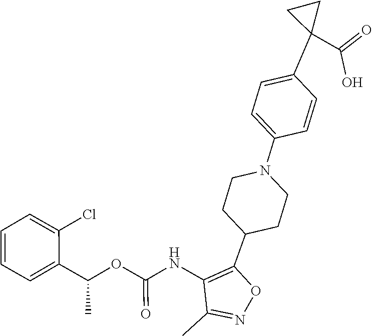 [SMILES: Cc1noc(C2CCN(c3ccc(C4(C(=O)O)CC4)cc3)CC2)c1NC(=O)O[C@H](C)c1ccccc1Cl] |
| 75 | 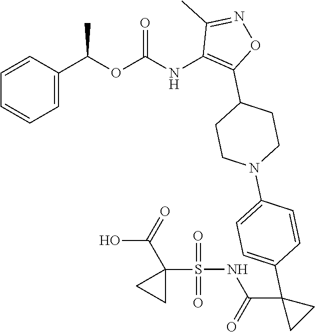 [SMILES: Cc1noc(C2CCN(c3ccc(C4(C(=O)NS(=O)(=O)C5(C(=O)O)CC5)CC4)cc3)CC2)c1NC(=O)O[C@H](C)c1ccccc1] |
| 76 | 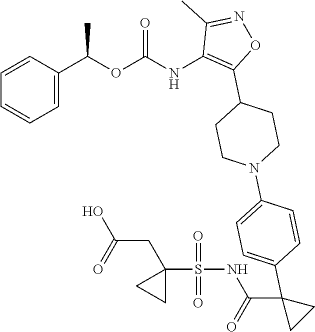 [SMILES: Cc1noc(C2CCN(c3ccc(C4(C(=O)NS(=O)(=O)C5(CC(=O)O)CC5)CC4)cc3)CC2)c1NC(=O)O[C@H](C)c1ccccc1] |
| 77 | 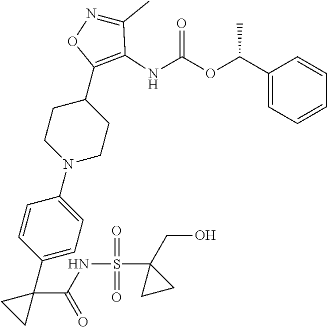 [SMILES: Cc1noc(C2CCN(c3ccc(C4(C(=O)NS(=O)(=O)C5(CO)CC5)CC4)cc3)CC2)c1NC(=O)O[C@H](C)c1ccccc1] |
| 78 | 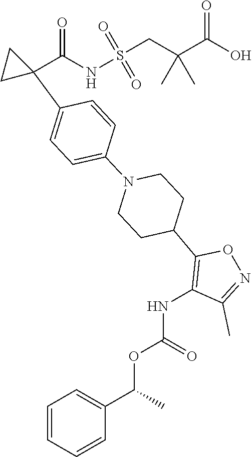 [SMILES: Cc1noc(C2CCN(c3ccc(C4(C(=O)NS(=O)(=O)CC(C)(C)C(=O)O)CC4)cc3)CC2)c1NC(=O)O[C@H](C)c1ccccc1] |
| 79 | 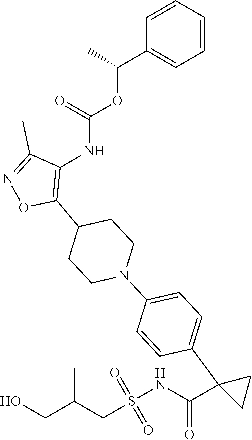 [SMILES: Cc1noc(C2CCN(c3ccc(C4(C(=O)NS(=O)(=O)CC(C)CO)CC4)cc3)CC2)c1NC(=O)O[C@H](C)c1ccccc1] |
| 80 | 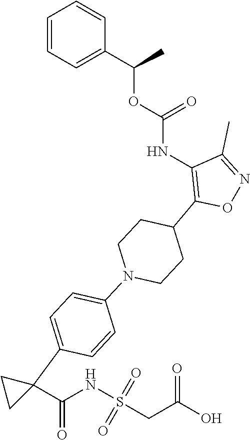 [SMILES: Cc1noc(C2CCN(c3ccc(C4(C(=O)NS(=O)(=O)CC(=O)O)CC4)cc3)CC2)c1NC(=O)O[C@H](C)c1ccccc1] |
| 81 | 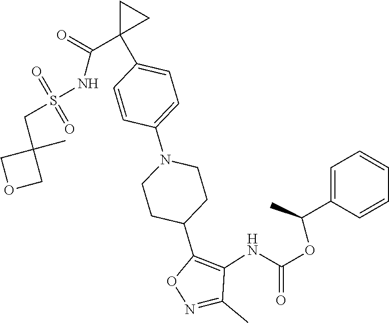 [SMILES: Cc1noc(C2CCN(c3ccc(C4(C(=O)NS(=O)(=O)CC5(C)COC5)CC4)cc3)CC2)c1NC(=O)O[C@@H](C)c1ccccc1] |
| 82 | 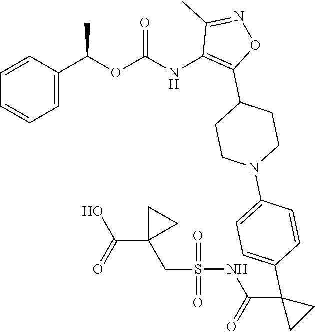 [SMILES: Cc1noc(C2CCN(c3ccc(C4(C(=O)NS(=O)(=O)CC5(C(=O)O)CC5)CC4)cc3)CC2)c1NC(=O)O[C@H](C)c1ccccc1] |
| 83 | 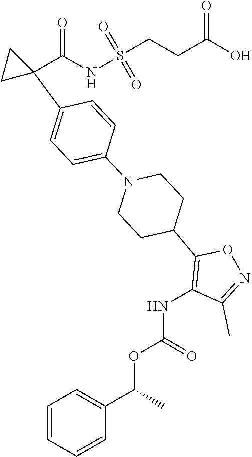 [SMILES: Cc1noc(C2CCN(c3ccc(C4(C(=O)NS(=O)(=O)CCC(=O)O)CC4)cc3)CC2)c1NC(=O)O[C@H](C)c1ccccc1] |
| 84 | 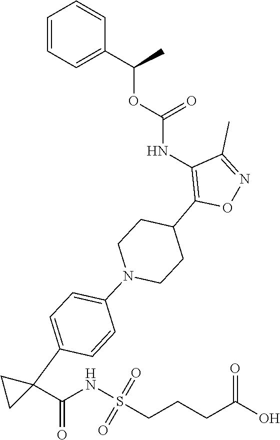 [SMILES: Cc1noc(C2CCN(c3ccc(C4(C(=O)NS(=O)(=O)CCCC(=O)O)CC4)cc3)CC2)c1NC(=O)O[C@H](C)c1ccccc1] |
| 85 | 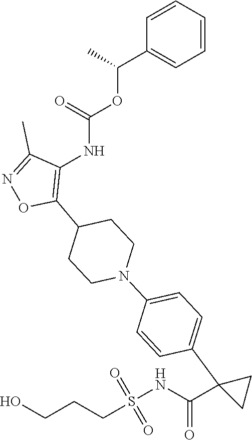 [SMILES: Cc1noc(C2CCN(c3ccc(C4(C(=O)NS(=O)(=O)CCCO)CC4)cc3)CC2)c1NC(=O)O[C@H](C)c1ccccc1] |
| 86 | 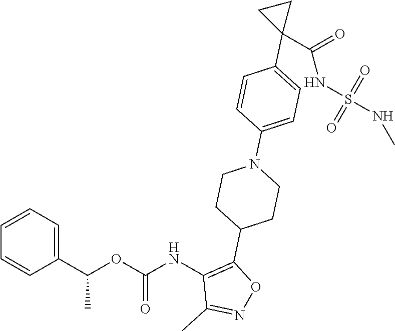 [SMILES: CNS(=O)(=O)NC(=O)C1(c2ccc(N3CCC(c4onc(C)c4NC(=O)O[C@H](C)c4ccccc4)CC3)cc2)CC1] |
| 87 | 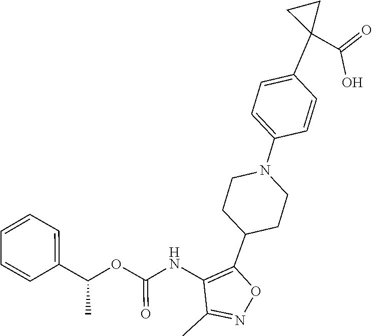 [SMILES: Cc1noc(C2CCN(c3ccc(C4(C(=O)O)CC4)cc3)CC2)c1NC(=O)O[C@H](C)c1ccccc1] |
| 88 | 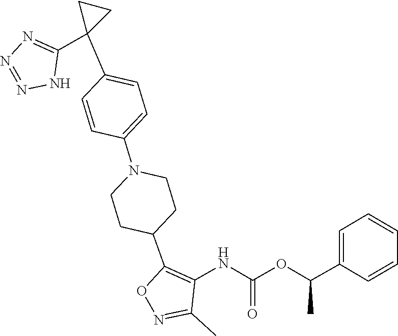 [SMILES: Cc1noc(C2CCN(c3ccc(C4(c5nnn[nH]5)CC4)cc3)CC2)c1NC(=O)O[C@H](C)c1ccccc1] |
| 89 | 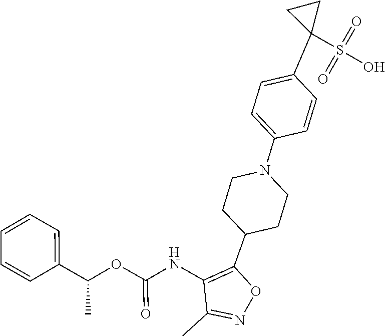 [SMILES: Cc1noc(C2CCN(c3ccc(C4(S(=O)(=O)O)CC4)cc3)CC2)c1NC(=O)O[C@H](C)c1ccccc1] |
| 90 | 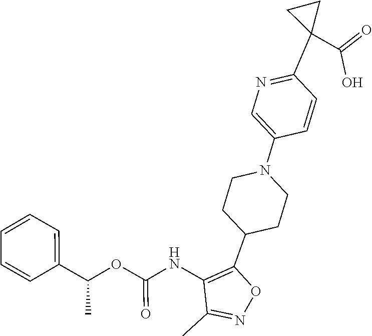 [SMILES: Cc1noc(C2CCN(c3ccc(C4(C(=O)O)CC4)nc3)CC2)c1NC(=O)O[C@H](C)c1ccccc1] |
| 91 | 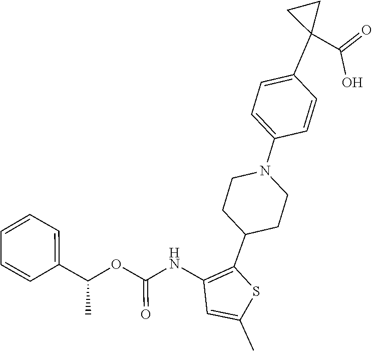 [SMILES: Cc1cc(NC(=O)O[C@H](C)c2ccccc2)c(C2CCN(c3ccc(C4(C(=O)O)CC4)cc3)CC2)s1] |
| 92 | 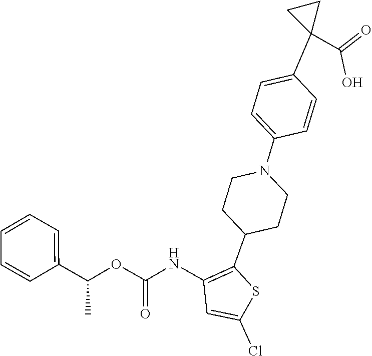 [SMILES: C[C@@H](OC(=O)Nc1cc(Cl)sc1C1CCN(c2ccc(C3(C(=O)O)CC3)cc2)CC1)c1ccccc1] |
| 93 | 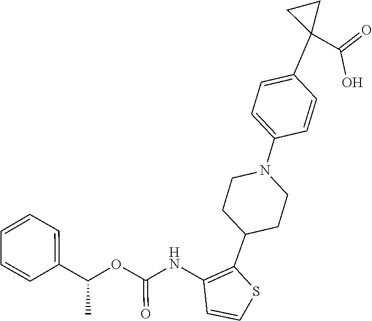 [SMILES: C[C@@H](OC(=O)Nc1ccsc1C1CCN(c2ccc(C3(C(=O)O)CC3)cc2)CC1)c1ccccc1] |
| 94 | 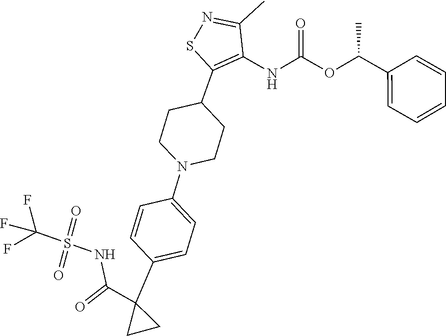 [SMILES: Cc1nsc(C2CCN(c3ccc(C4(C(=O)NS(=O)(=O)C(F)(F)F)CC4)cc3)CC2)c1NC(=O)O[C@H](C)c1ccccc1] |
| 95 | 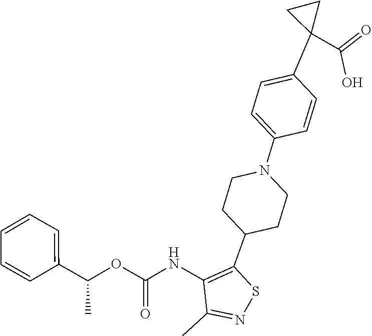 [SMILES: Cc1nsc(C2CCN(c3ccc(C4(C(=O)O)CC4)cc3)CC2)c1NC(=O)O[C@H](C)c1ccccc1] |
| 96 | 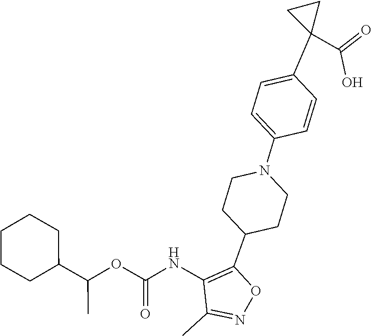 [SMILES: Cc1noc(C2CCN(c3ccc(C4(C(=O)O)CC4)cc3)CC2)c1NC(=O)OC(C)C1CCCCC1] |
| 97 | 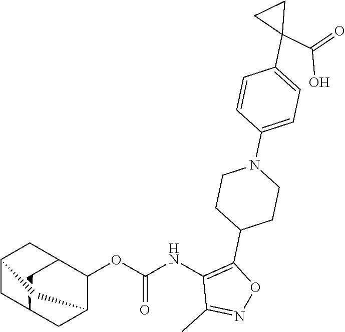 [SMILES: Cc1noc(C2CCN(c3ccc(C4(C(=O)O)CC4)cc3)CC2)c1NC(=O)OC1C2C[C@H]3C[C@@H](C2)C[C@@H]1C3] |

### Pharmaceutical Compositions

Also provided herein is a pharmaceutical composition comprising a compound of Formula (I), or a pharmaceutically acceptable salt thereof, and a pharmaceutically acceptable carrier.

### Methods of Treatment

### Indications

Provided herein are methods for antagonizing the activation of the lysophosphatidic acid receptor 1 (LPA1). For example, provided herein are LPA1 antagonists useful for treating a fibrotic in a subject.

Some embodiments provide a method of treating a fibrotic disease in a subject in need thereof, comprising administering to the subject a therapeutically effective amount of a compound of Formula (I), or a pharmaceutically acceptable salt thereof, or a pharmaceutical composition as described herein.

Some embodiments provide a method of treating a fibrotic disease in a subject previously determined to have a fibrotic disease, comprising administering to the subject a therapeutically effective amount of a compound of Formula (I), or a pharmaceutically acceptable salt thereof, or a pharmaceutical composition as described herein.

Some embodiments provide a method of treating a fibrotic disease in a subject comprising
(a) determining that the subject has a fibrotic disease; and
(b) administering to the subject a therapeutically effective amount of a compound of Formula (I), or a pharmaceutically acceptable salt thereof, or a pharmaceutical composition as described herein.

In some embodiments, the subject is suspected of having a fibrotic disease.

In some embodiments, the subject is at risk of developing a fibrotic disease.

In some embodiments, the fibrotic disease is associated with one or more of the liver, lung, kidney, heart, eyes, skin, pancreas, intestines, or bladder. In some embodiments, the fibrotic disease is associated with cell proliferative disease such as cancer. In some embodiments, the fibrotic disease is associated with nerve injury. In some embodiments, the fibrotic disease is associated with tissue repair.

In some embodiments, the fibrotic disease comprises liver fibrosis. In some embodiments, the liver fibrosis is associated with, for example, acute hepatitis, chronic hepatitis, liver cirrhosis, alcohol and drug induced liver fibrosis, fatty liver (NAFLD), non-alcoholic steatohepatitis (NASH), viral hepatitis, hepatic blood flow disorder, and/or portal hypertension. In some embodiments, the fibrotic disease is liver fibrosis.

In some embodiments, the fibrotic disease comprises lung fibrosis. In some embodiments, the lung fibrosis is associated with, for example, idiopathic pulmonary fibrosis, interstitial lung disease (ILD), radiation induced lung injury, and/or acute respiratory distress syndrome (ARDS). In some embodiments, the fibrotic disease is lung fibrosis.

In some embodiments, the fibrotic disease comprises skin fibrosis. In some embodiments, the skin fibrosis is associated with, for example, scleroderma, keloids, and/or Dupuytren's contracture. In some embodiments, the fibrotic disease is skin fibrosis.

In some embodiments, the fibrotic disease comprises kidney fibrosis. In some embodiments, the kidney fibrosis is associated with, for example, chronic kidney disease, Alport syndrome, and/or hypertension. In some embodiments, the fibrotic disease is kidney fibrosis.

In some embodiments, the fibrotic disease comprises nerve injury. In some embodiments, the nerve injury is associated with, for example, neuropathic pain and/or multiple sclerosis, demyelination, or fetal hydrocephalus. In some embodiments, the fibrotic disease is nerve injury.

In some embodiments, the fibrotic disease comprises a cell proliferative disorder. In some embodiments, the cell proliferative disorder is, for example, a hematological or solid tumor, including cancer metastases. In some embodiments, the fibrotic disease is a cell proliferative disorder.

In some embodiments, the fibrotic disease comprises intestinal fibrosis. In some embodiments, the fibrotic disease comprises head and neck fibrosis. In some embodiments, the fibrotic disease comprises bladder fibrosis. In some embodiments, the fibrotic disease comprises biliary cirrhosis. In some embodiments, the fibrotic disease is intestinal fibrosis. In some embodiments, the fibrotic disease is head and neck fibrosis. In some embodiments, the fibrotic disease is bladder fibrosis. In some embodiments, the fibrotic disease is biliary cirrhosis.

Some embodiments provide a method for reducing LPA1 activation in a subject, comprising administering to the subject a therapeutically effective amount of a compound of Formula (I), or a pharmaceutically acceptable salt thereof.

Some embodiments provide a method for reducing LPA1 activation in a cell comprising LPA1, the method comprising contacting the cell with a compound of Formula (I), or a pharmaceutically acceptable salt thereof. In some embodiments, the contacting is in vitro. In some embodiments, the contacting is in vivo. In some embodiments, the contacting comprises administering to a subject having the cell a therapeutically effective amount of a compound of Formula (I), or a pharmaceutically acceptable salt thereof.

The ability of test compounds to act as LPA1 antagonists may be demonstrated by assays known in the art. The activity of the compounds and compositions provided herein as LPA1 antagonists can be assayed in vitro, in vivo, or in a cell line.

Potency of an LPA1 antagonist as provided herein can be determined by EC₅₀ or IC₅₀ values. A compound with a lower EC₅₀ or IC₅₀ value, as determined under substantially similar conditions, is a more potent antagonist relative to a compound with a higher EC₅₀ or IC₅₀ value.

When employed as pharmaceuticals, the compounds of Formula (I), including pharmaceutically acceptable salts thereof, can be administered in the form of pharmaceutical compositions as described herein.

### Examples

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the schemes provided herein, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

### Synthetic Examples

### Example 1. Preparation of 1-[4-[4-[3-methyl-4-[[(1R)-1-phenylethoxy]carbonylamino]isoxazol-5-yl]-1-piperidyl]phenyl]cyclopropanesulfonic acid (Compound 89)

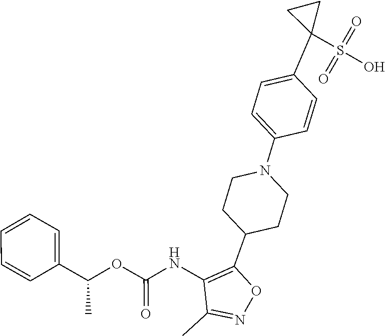 [SMILES: Cc1noc(C2CCN(c3ccc(C4(S(=O)(=O)O)CC4)cc3)CC2)c1NC(=O)O[C@H](C)c1ccccc1]

### Step 1. Preparation of Ethyl (4-bromophenyl) methanesulfonate

To a solution of (4-bromophenyl)methanesulfonyl chloride (2 g, 7.42 mmol, 1 eq) in DCM (20 mL) was added sodium; ethanolate (3.79 g, 11.13 mmol, 20% purity, 1.5 eq) at 0 °C, The reaction solution was stirred at 0 °C for 1 hr. TLC (PE:EA=5:1, Rf=0.3) showed starting material consumed and a new major spot was detected. The residue was dissolved in water (50 mL) and subsequently extracted with CH₂Cl₂ (3×50 mL). The CH₂Cl₂ layer was dried (Na₂SO₄), and evaporated under vacuum. The residue was purified by silical gel column chromatography (PE:EA=5:1) to get ethyl (4-bromophenyl) methanesulfonate (1.8 g, 6.45 mmol, 86.90% yield) as a white solid. ¹H NMR (400 MHz, chloroform-d) δ=7.59-7.53 (m, 2H), 7.34-7.29 (m, 2H), 4.31 (s, 2H), 4.18 (q, J=7.2 Hz, 2H), 1.34 (t, J=7.2 Hz, 3H).

### Step 2. Preparation of Ethyl 1-(4-bronophenyl) cyclopropanesulfonate

To a solution of ethyl (4-bromophenyl) methanesulfonate (1 g, 3.58 mmol, 1 eq) in THF (30 mL) was added NaHMDS (1 M, 10 mL, 2.79 eq) at −40 °C and stirred for 1 hr, then the resulting mixture was added 1,3,2-dioxathiolane 2,2-dioxide(2A) (1.00 g, 8.06 mmol, 2.25 eq) at −40 °C and then stirred for 1 hr. TLC (PE/EA=5:1, Rf=0.4) showed a new spot was detected. The reaction mixture was partitioned between EtOAc (130 mL) and NH₄Cl (80 mL), washed with brine (90 mL), dried over Na₂SO₄, filtered and evaporated under reduced pressure to give the crude product. The crude product was purified by column chromatography on silica gel and eluted with PE/EA=50:1 to 5:1 and evaporated under recuded pressure to give the ethyl 1-(4-bromophenyl) cyclopropanesulfonate (400 mg, 1.31 mmol, 36.59% yield) as a white solid.

### Step 3. Preparation of 1-(4-bromophenyl) cyclopropanesulfonic acid

To a solution of ethyl 1-(4-bromophenyl) cyclopropanesulfonate (250 mg, 819.17 mol, 1 eq) in EtOH (5 mL)/H₂O (2.5 mL) was added KOH (140 mg, 2.50 mmol, 3.05 eq). The reaction solution was stirred at 50 °C for 2 hr. TLC (PE:EA=2:1, Rf=0.02) showed starting material consumed and a new major spot was detected. LCMS showed desired MS was detected. The pH of the residue was adjusted to 6 by adding AcOH. The reaction mixture was partitioned between EtOAc (20 mL) and water (10 mL), washed with brine (10 mL), dried over Na₂SO₄, filtered and evaporated under reduced pressure to give the crude product. The crude product was lyophilized to give 1-(4-bromophenyl) cyclopropanesulfonic acid (100 mg, 353.62 umol, 43.17% yield, 98% purity) as a brown solid. The crude product was used for next step without further purification. MS m/z: 274.9 ({⁷⁹Br})[M−H]⁻

### Step 4. Preparation of 1-[4-[4-[3-methyl-4-[[(1R)-1-phenylethoxy]carbonylamino]isoxazol-5-yl]-1-piperidyl]phenyl]cyclopropanesulfonic acid

To a mixture of 1-(4-bromophenyl) cyclopropanesulfonic acid (100 mg, 360.84 mol, 1 eq) in dioxane (1 mL) was added RuPhos Pd G₃ (100 mg, 119.57 umol, 3.31e−1 eq), Cs₂CO₃ (200 mg, 613.84 umol, 1.70 eq) and [(1R)-1-phenylethyl]N-[3-methyl-5-(4-piperidyl) isoxazol-4-yl]carbamate (160 mg, 485.74 mol, 1.35 eq), The reaction solution was degassed and purged under N₂ for three times, The reaction solution was stirred at 80 °C for 1 hr. LCMS showed desired MS was detected. The pH of the residue was adjusted to 6 by adding AcOH. The residue was purified by Prep-HPLC (column: Shim-pack C₁₈ 150*25*10 um; mobile phase: [water (FA)-ACN]; B %: 25%-55%, 10 min), After Prep-HPLC purification, the eluent was concentrated to remove organic solvents. The residual aqueous solution was lyophilized to give 1-[4-[4-[3-methyl-4-[[(1R)-1-phenylethoxy]carbonylamino]isoxazol-5-yl]-1-piperidyl]phenyl]cyclopropanesulfonic acid (2 mg, 3.81 mol, 1.05% yield, 100% purity) as a white solid. ¹H NMR (400 MHz, methanol-d₄) δ=7.54 (d, J=7.6 Hz, 2H), 7.43-7.28 (m, 5H), 7.07-6.95 (m, 2H), 5.91-5.71 (m, 1H), 3.70 (m, 2H), 3.01-2.77 (m, 3H), 2.13 (s, 3H), 1.98 (s, 4H), 1.60-1.52 (m, 5H), 1.04-0.98 (m, 2H). MS m/z: 524.1[M−H]⁻

### Example 2. Preparation of [(1R)-1-phenylethyl]N-[3-methyl-5-[1-[4-[1-(1H-tetrazol-5-yl) cyclopropyl]phenyl]-4-piperidyl]isoxazol-4-yl]carbamate (Compound 88)

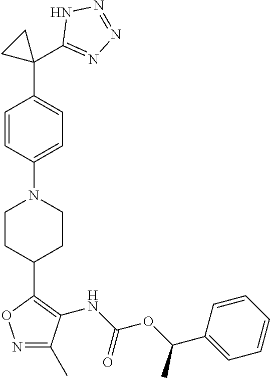 [SMILES: Cc1noc(C2CCN(c3ccc(C4(c5nnn[nH]5)CC4)cc3)CC2)c1NC(=O)O[C@H](C)c1ccccc1]

### Step 1. Preparation of 1-(4-bromophenyl) cyclopropanecarbonitrile

To a solution of 2-(4-bromophenyl) acetonitrile (10 g, 51.01 mmol, 1 eq) and 1-bromo-2-chloro-ethane (10 g, 69.73 mmol, 5.78 mL, 1.37 eq) in DMF (200 mL) was added Cs₂CO₃ (50 g, 153.46 mmol, 3.01 eq) and benzyl (triethyl) ammonium; chloride (6 g, 26.34 mmol, 5.16e−1 eq), The reaction solution was stirred at 110 °C for 12 hr. TLC (PE:EA=10:1, Rf=0.52) showed starting material consumed and a new major spot was detected. The reaction mixture was partitioned between H₂O (500 mL) and EtOAc (500 mL). The organic phase was separated, washed with sat. NaCl (500 mL*3), dried over anhydrous Na₂SO₄, filtered and concentrated under reduced pressure to give a residue. 1-(4-bromophenyl) cyclopropanecarbonitrile (10 g, 45.03 mmol, 88.28% yield) as a red oil was used for next step without further purification. ¹H NMR (400 MHz, CDCl₃) δ (ppm): 7.50-7.43 (m, 2H), 7.20-7.11 (m, 2H), 1.77-1.70 (m, 2H), 1.41-1.35 (m, 2H).

### Step 2. Preparation of [(1R)-1-phenylethyl]N-[5-[I-[4-(1-cyanocyclopropyl) phenyl]-4-piperidyl]-3-methyl-isoxazol-4-yl]carbamate

To a solution of [(1R)-1-phenylethyl]N-[3-methyl-5-(4-piperidyl) isoxazol-4-yl]carbamate (200 mg, 607.18 mol, 1 eq) and 1-(4-bromophenyl) cyclopropanecarbonitrile (170 mg, 765.49 mol, 1.26 eq) in dioxane (5 mL) was added RuPhos Pd G₃ (200 mg, 239.13 mol, 3.94e−1 eq) and Cs₂CO₃ (400 mg, 1.23 mmol, 2.02 eq), The reaction solution was degassed and purged under N₂ for three times, the reaction mixture was stirred at 80 °C for 1 hr. LCMS showed desired MS was detected. TLC (PE/EtOAc=3/1, Rf=0.20) showed one major spot was observed The reaction was extracted with EtOAc (100 mL*3). The combined organic phase was washed with sodium bicarbonate (100 mL*3) and saturated brine (100 mL*2), dried over anhydrous Na₂SO₄, filtered and concentrated in vacuo to give a residual. The crude product was purified by prep-TLC with PE:EtOAc=3:1 to give [(1R)-1-phenylethyl]-N-[5-[1-[4-(1-cyanocyclopropyl)phenyl]-4-piperidyl]-3-methyl-isoxazol-4-yl]carbamate (260 mg, 363.01 umol, 59.79% yield, 65.7% purity) as yellow oil.

### Step 3. Preparation of[(1R)-1-phenylethyl]N-[3-methyl-5-[1-[4-[1-(1H-tetrazol-5-yl) cyclopropyl]phenyl]-4-piperidyl]isoxazol-4-yl]carbamate

To a solution of [(1R)-1-phenylethyl]-N-[5-[1-[4-(1-cyanocyclopropyl) phenyl]-4-piperidyl]-3-methyl-isoxazol-4-yl]carbamate (200 mg, 425.02 umol, 1 eq) and NH₄Cl (100 mg, 1.87 mmol, 4.40 eq) in DMSO (2 mL) was added NaN₃ (217 mg, 3.34 mmol, 7.85 eq), The reaction solution was stirred at 110 °C for 2 hr. LCMS showed desired MS was detected. The pH of the reaction solution was adjusted to 9 by adding sat NaHCO₃. The residue was partitioned between H₂O (10 mL) and EtOAc (10*3 mL). The organic layer was dried over anhydrous Na₂SO₄, filtered and concentrated under reduced pressure to give a residue. The residue was purified by prep-HPLC (column: Unisil 3-100 C₁₈ Ultra 150*50 mm*3 um; mobile phase: [water (0.225% FA)-ACN]; B %: 43%-63%, 10 min). After Prep-HPLC purification, the eluent was concentrated to remove organic solvents. The residual aqueous solution was lyophilized to give [(1R)-1-phenylethyl]N-[3-methyl-5-[1-[4-[1-(1H-tetrazol-5-yl)cyclopropyl]phenyl]-4-piperidyl]isoxazol-4-yl]carbamate (10 mg, 19.47 mol, 4.58% yield, 100% purity) as a white solid. ¹H NMR (400 MHz, methanol-d₄) δ (ppm): 7.51-7.30 (m, 4H), 7.30-7.26 (m, 3H), 6.98 (d, J=8.6 Hz, 2H), 5.84-5.73 (m, 1H), 3.72 (d, J=12.8 Hz, 2H), 2.97-2.68 (m, 3H), 2.10 (s, 3H), 1.98-1.83 (m, 4H), 1.61-1.53 (m, 5H), 1.47-1.41 (m, 2H). MS m/z: 512.2[M−H]⁻.

### Example 3. Preparation of methyl 2-methyl-2-[[1-[4-[4-[3-methyl-4-[[(1R)-1-phenylethoxy]carbonylamino]isoxazol-5-yl]-1-piperidyl]phenyl]cyclopropanecarbonyl]sulfamoyl]propanoate (Compound 69)

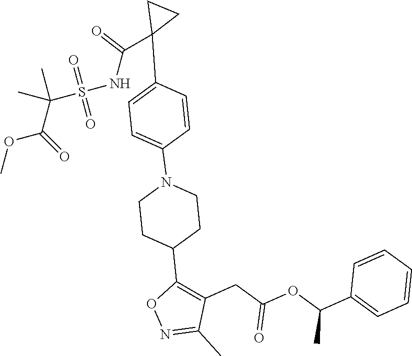 [SMILES: COC(=O)C(C)(C)S(=O)(=O)NC(=O)C1(c2ccc(N3CCC(c4onc(C)c4CC(=O)O[C@H](C)c4ccccc4)CC3)cc2)CC1]

### Step 1. Preparation of methyl 2-[bis[(4-methoxyphenyl)methyl]sulfamoyl]acetate

To a solution of methyl 2-chlorosulfonylacetate (500.00 mg, 2.90 mmol, 7.45e−1 eq) in DCM (15 mL) was added 1-(4-methoxyphenyl)-N-[(4-methoxyphenyl)methyl]methanamine (1 g, 3.89 mmol, 1 eq), DIPEA (1.48 g, 11.48 mmol, 2.00 mL, 2.95 eq) at 0 °C, the resulting mixture was stirred at 25 °C for 12 hr. TLC (PE/EA=3:1, Rf=0.4) showed a new spot was detected and the 1-(4-methoxyphenyl)-N-[(4-methoxyphenyl)methyl]methanamine consumed. The reaction mixture was partitioned between DCM (70 mL) and sat·NaHCO₃ (50 mL), washed with brine (90 mL), dried over Na₂SO₄, filtered and evaporated under reduced pressure to give the crude product. The crude product was purified by silica gel column chromatography and eluted with PE/EA=50:1 to 3:1 to give the methyl 2-[bis[(4-methoxyphenyl)methyl]sulfamoyl]acetate (500 mg, 1.27 mmol, 32.70% yield) as a yellow solid.

¹H NMR (400 MHz, DMSO-d₆) δ=7.13 (d, J=8.8 Hz, 4H), 6.85 (d, J=8.8 Hz, 4H), 4.33-4.27 (m, 2H), 4.24 (s, 4H), 3.72 (s, 6H), 3.69 (s, 3H).

### Step 2. Preparation of methyl2-[bis[(4-methoxyphenyl)methyl]sulfamoyl]-2-methyl-propanoate

To a solution of methyl 2-[bis[(4-methoxyphenyl) methyl]sulfamoyl]acetate (400 mg, 1.02 mmol, 1 eq) in DMF (10 mL) was added K₂CO₃ (480.00 mg, 3.47 mmol, 3.42 eq), MeI (684.00 mg, 4.82 mmol, 0.3 mL, 4.74 eq), then the resulting mixture was stirred at 40 °C for 48 hr. LCMS showed the desired MS was detected and the methyl 2-[bis[(4-methoxyphenyl) methyl]sulfamoyl]acetate consumed. TLC (PE/EA=3:1, Rf=0.7) showed a new spot was detected and the methyl 2-[bis[(4-methoxyphenyl) methyl]sulfamoyl]acetate consumed. The reaction mixture was partitioned between EtOAc (100 mL) and water (70 mL), washed with brine (60 mL), dried over Na₂SO₄, filtered and evaporated under reduced pressure to give the crude product. The crude product was purified by prep-TLC (PE/EA=3:1, Rf=0.7) to give the methyl 2-[bis[(4-methoxyphenyl) methyl]sulfamoyl]-2-methyl-propanoate (250 mg, 593.11 mol, 58.34% yield) as a white solid.

¹H NMR (400 MHz, chloroform-d) δ=7.10 (d, J=8.6 Hz, 4H), 6.79 (d, J=8.6 Hz, 4H), 4.29 (s, 4H), 3.79 (s, 9H), 3.78 (s, 6H), 1.75 (s, 6H).

### Step 3. Preparation of methyl 2-methyl-2-sulfamoyl-propanoate

To a solution of methyl 2-[bis[(4-methoxyphenyl) methyl]sulfamoyl]-2-methyl-propanoate (100 mg, 237.24 umol, 1 eq) in DCM (2 mL) was added TFA (0.5 mL), then the resulting mixture was stirred at 25 °C for 1 hr. TLC (PE/EA=3:1, Rf=0.1, I₂) showed a new spot was detected and the methyl 2-[bis[(4-methoxyphenyl) methyl]sulfamoyl]-2-methyl-propanoate consumed. The reaction mixture was filtered and the filtrate was evaporated under reduced pressure to give the crude methyl 2-methyl-2-sulfamoyl-propanoate (80 mg, crude) as white solid which was used into the step without further purification.

### Step 4. Preparation of methyl 2-methyl-2-[[1-[4-[4-[3-methyl-4-[[(1R)-1-phenylethoxy]carbonylamino]isoxazol-5-yl]-1-piperidyl]phenyl]cyclopropanecarbonyl]sulfamoyl]propanoate

To a solution of 1-[4-[4-[3-methyl-4-[[(1R)-1-phenylethoxy]carbonylamino]isoxazol-5-yl]-1-piperidyl]phenyl]cyclopropane carboxylic acid (80 mg, 163.41 mol, 1 eq) in DCM (2 mL) was added methyl 2-methyl-2-sulfamoyl-propanoate (70 mg, 386.29 umol, 2.36 eq), DMAP (50 mg, 409.27 umol, 2.50 eq) and EDCI (50 mg, 260.82 mol, 1.60 eq), then the resulting mixture was stirred at 25 °C for 12 hr. LCMS showed the desired MS was detected and the 1-[4-[4-[3-methyl-4-[[(1R)-1-phenylethoxy]carbonylamino]isoxazol-5-yl]-1-piperidyl]phenyl]cyclopropanecarboxylic acid consumed. The reaction mixture was partitioned between EtOAc (70 mL) and water (50 mL), washed with brine (50 mL), dried over Na₂SO₄, filtered and evaporated under reduced pressure to give the crude product. The crude product was purified by Prep-HPLC (UniSil 3-100 C₁₈ Ultra (150*25 mm*3 um); mobile phase: [water (FA)-ACN]; B %: 47%-77%, 7 min) and lyophilized to give the methyl 2-methyl-2-[[1-[4-[4-[3-methyl-4-[[(1R)-1-phenylethoxy]carbonylamino]isoxazol-5-yl]-1-piperidyl]phenyl]cyclopropanecarbonyl]sulfamoyl]propanoate (5.7 mg, 8.56 umol, 5.24% yield, 98% purity) as a yellow solid.

¹H NMR (400 MHz, methanol-d₄) δ=7.43-7.26 (m, 7H), 7.01 (d, J=8.6 Hz, 2H), 5.80-5.75 (m, 1H), 3.76-3.73 (m, 2H), 3.70 (s, 3H), 2.98-2.86 (m, 1H), 2.82-2.71 (m, 2H), 2.11 (s, 3H), 1.97-1.85 (m, 4H), 1.64-1.55 (m, 9H), 1.53-1.50 (m, 2H), 1.20-1.17 (m, 2H). MS m/z: 653.2 [M+H]⁺

### Example 4. Preparation of (R)-1-(3-methoxy-4-(4-(3-methyl-4-(((1-phenylethoxy) carbonyl)amino)isoxazol-5-yl)piperidin-1-yl)phenyl)cyclopropane-1-carboxylic acid (Compound 63)

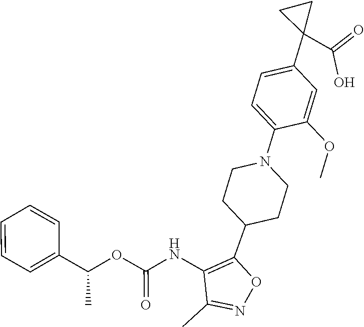 [SMILES: COc1cc(C2(C(=O)O)CC2)ccc1N1CCC(c2onc(C)c2NC(=O)O[C@H](C)c2ccccc2)CC1]

### Step 1. Preparation of methyl 1-(4-bromo-3-methoxyphenyl)cyclopropane-1-carhoxylate

To a stirred solution of 1-(4-bromo-3-methoxyphenyl)cyclopropane-1-carboxylic acid (0.500 g, 1.84 mmol) in methanol (0.1 mL) added sulfuric acid (0.1 mL, 1.84 mmol) at 0 °C, stirred for 10 minutes, maintained reaction mixture at 70 °C for 8h. Progress of the reaction was monitored by TLC and LCMS. After completion of reaction, reaction mixture was concentrated, diluted with water (15 mL) extracted with ethyl acetate (2×15 mL). The combined organic layer was washed with water (2×10 mL), 10% NaHCO₃ solution (2×7 mL) and brine solution (5 mL), dried over anhydrous sodium sulphate, filtered and evaporated under reduced pressure to afford methyl 1-(4-bromo-3-methoxyphenyl) cyclopropane-1-carboxylate (0.5 g and 95% Yield) as a colorless liquid; MS (ES) m/z 287.0 (M+H)⁺.

### Step 2. Preparation of 5-(1-(2-methoxy-4-(1-(methoxycarbonyl) cyclopropyl)phenyl) piperidin-4-yl)-3-methylisoxazole-4-carboxylic acid

To a stirred solution of 3-methyl-5-(piperidin-4-yl)-1,2-oxazole-4-carboxylic acid hydrochloride (0.4 g, 1.62 mmol) in dimethylformamide (13.2 mL, 170 mmol) were added cesium carbonate (2.11 g, 4 eq., 6.49 mmol), methyl 1-(4-bromo-3-methoxyphenyl) cyclopropane-1-carboxylate (509 mg, 1.1 eq., 1.62 mmol) and X-phos (77.3 mg, 0.1 eq., 162 umol), purged under argon gas for 15 minutes. Finally added Pd₂(dba)₃ (74.2 mg, 0.05 eq., 81.1 umol) and heated reaction mixture to 100 °C for 16 hours in sealed tube. Progress of the reaction was monitored by TLC and LCMS. After completion of reaction, reaction mass was cooled to room temperature and filtered through celite, and concentrated then diluted with water (20 mL) and washed with ethyl acetate, then Aq. layer was acidified with 10% citric acid until pH was acidic, then extracted with ethyl acetate (2×100 mL), washed with water (50 mL), brine solution (50 mL), dried over anhydrous sodium sulphate, filtered and evaporated under reduced pressure to afford 5-(1-{4-[1-(methoxycarbonyl)cyclopropyl]phenyl}piperidin-4-yl)-3-methyl-1,2-oxazole-4-carboxylic acid (150 mg, Yield 22%%) as a dark yellow solid; MS (ES) m/z 415.0 (M+1H)⁺

### Step 3. Preparation of methyl (R)-1-(3-methoxy-4-(4-(3-methyl-4-(((1-phenylethoxy) carbonyl)amino)isoxazol-5-yl)piperidin-1-yl)phenyl)cyclopropane-1-carboxylate

To a stirred solution of 5-(1-{4-[1-(methoxycarbonyl)cyclopropyl]phenyl}piperidin-4-yl)-3-methyl-1,2-oxazole-4-carboxylic acid (0.100 g, 241 umol) in toluene (10 mL) were added triethylamine (0.1 mL, 3 eq., 719 umol) and ((1R)-1-phenylethan-1-ol (29.5 mg, 1 eq. 241 umol) heated reaction mixture to 50 °C, for 20 minutes. Then added DPPA (57.5 μL, 1.1 eq., 265 umol), heated to 85 °C, maintained for 4 hours. Progress of the reaction was monitored by TLC and LCMS. After completion of reaction, reaction mass was diluted with water (20 mL) extracted with ethyl acetate (2×25 mL). The combined organic layer was washed with water (2×20 mL), brine solution (20 mL), dried over anhydrous sodium sulphate, filtered and evaporated under reduced pressure to get crude product. The crude product was purified by combiflash MPLC using ethyl acetate in n-hexane (1% to 28%) to afford methyl (R)-1-(3-methoxy-4-(4-(3-methyl-4-(((1-phenylethoxy) carbonyl)amino)isoxazol-5-yl)piperidin-1-yl)phenyl)cyclopropane-1-carboxylate (50 mg, Yield: 39%) as a pale yellow gum; MS (ES) m/z 533.3 (M+H)⁺.

### Step 4. Preparation of (R)-1-(3-methoxy-4-(4-(3-methyl-4-(((1-phenylethoxy) carbonyl)amino)isoxazol-5-yl)piperidin-1-yl)phenyl)cyclopropane-1-carboxylic acid

To a stirred solution of methyl 1-(3-methoxy-4-{4-[3-methyl-4-({[(1R)-1-phenylethoxy]carbonyl}amino)-1,2-oxazol-5-yl]piperidin-1-yl}phenyl)cyclopropane-1-carboxylate (50 mg, 93.7 umol) in THF (3.0 mL), MeOH (3.0 mL) solution was added lithium(1+) hydrate hydroxide (15.7 mg, 375 umol) in water (3.0 mL) and the mixture was stirred at RT for 18 hours. The progress of the reaction was monitored by LCMS and TLC. After completion of the reaction, the reaction mixture was concentrated, diluted with water (10 mL), washed with diethyl ether (2×5 mL), pH was adjusted with 10% Aq. citric acid up to 3 to 4, white solid was precipitated, filtered on Buchner funnel, solid was washed with water (2×5 mL), pentane (2×5 mL) and dried under vacuum to get crude product (30 mg). It was purified by Prep HPLC to afford 1-(3-methoxy-4-{4-[3-methyl-4-({[(1R)-1-phenylethoxy]carbonyl}amino)-1,2-oxazol-5-yl]piperidin-1-yl}phenyl)cyclopropane-1-carboxylic acid as a white solid (10 mg, 20% yield); MS (ES) m/z 520.3 (M+1H)⁺. LC purity 98.72% @254 nm. ¹H NMR (400 MHz, DMSO-d₆): δ 12.2 (s, 1H), 8.92 (s, 1H), 7.39-7.30 (m, 5H), 6.85 (d, J=7.6 Hz, 1H), 6.80 (d, J=8.4 Hz, 2H), 5.75 (q, J=6.8 Hz, 1H), 3.78 (s, 3H), 3.38-3.35 (m, 2H), 2.84-2.82 (m, 1H), 2.58-2.56 (m, 2H), 2.04 (s, 3H), 1.85-1.83 (m, 4H), 1.53-1.51 (m, 2H), 1.41-1.39 (m, 2H), 1.11 (d, J=2.8 Hz, 2H).

### Example 5. Preparation of 1-(3-methoxy-4-{6-[3-methyl-4-({[(1R)-1-phenylethoxy]carbonyl}amino)-1,2-oxazol-5-yl]-2-azaspiro[3.3]heptan-2-yl}phenyl)cyclopropane-1-carboxylic acid (Compound 46)

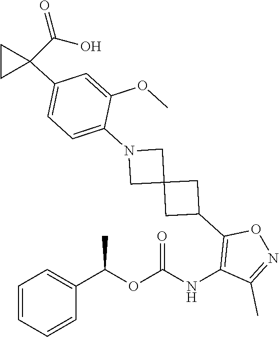 [SMILES: COc1cc(C2(C(=O)O)CC2)ccc1N1CC2(CC(c3onc(C)c3NC(=O)O[C@H](C)c3ccccc3)C2)C1]

### Step 1. Preparation of Methyl 1-(4-bromo-3-methoxyphenyl)cyclopropane-1-carboxylate

To a stirred solution of 1-(4-bromo-3-methoxyphenyl)cyclopropane-1-carboxylic acid (1 g, 3.69 mmol) in methanol (1 mL) added sulfuric acid (217 uL, 3.69 mmol) at 0 °C, stirred for 10 minutes, maintained reaction mixture at 70 °C for 8h. Progress of the reaction was monitored by TLC and LCMS. After completion of reaction, reaction mixture was concentrated, diluted with water (15 mL) extracted with ethyl acetate (2×15 mL). The combined organic layer was washed with water (2×10 mL), 10% NaHCO₃ solution (2×7 mL) and brine solution (5 mL), dried over anhydrous sodium sulphate, filtered and evaporated under reduced pressure to afford methyl 1-(4-bromo-3-methoxyphenyl)cyclopropane-1-carboxylate (0.8 g and 76% Yield) as colorless liquid MS (ES) m/z 285.0 (M+H)⁺.

### Step 2. Preparation of 5-(2-{2-methoxy-4-[1-(methoxycarbonyl)cyclopropyl]phenyl}-2-azaspiro[3.3]heptan-6-yl)-3-methyl-1,2-oxazole-4-carboxylic acid

To a stirred solution of 5-{2-azaspiro[3.3]heptan-6-yl}-3-methyl-1,2-oxazole-4-carboxylic acid hydrochloride (350 mg, 1.35 mmol) in dimethylformamide (8.75 mL, 113 mmol) were added Cesium carbonate (1.76 g, 4 eq., 5.41 mmol), methyl 1-(4-bromo-3-methoxyphenyl)cyclopropane-1-carboxylate (386 mg, 1.35 mmol) and X-phos (64.5 mg, 0.1 eq., 135 umol), purged under argon gas for 15 minutes. Finally added Pd₂(dba)₃ (61.9 mg, 0.05 eq., 67.6 umol) and heated reaction mixture to 100 °C for 16 hours inside sealed tube. Progress of the reaction was monitored by TLC and LCMS. After completion of reaction, reaction mass was cooled to room temperature and filtered through celite, and concentrated then diluted with water (20 mL) and washed with ether, then Aq. layer was acidified with 10% citric acid until pH is acidic, then extracted with ethyl acetate (2×20 mL), washed with water (20 mL), brine solution (20 mL), dried over anhydrous sodium sulphate, filtered and evaporated under reduced pressure to get crude. The crude product was co-distilled with toluene and given ether and pentane wash to afford 5-(2-{2-methoxy-4-[1-(methoxycarbonyl)cyclopropyl]phenyl}-2-azaspiro[3.3]heptan-6-yl)-3-methyl-1,2-oxazole-4-carboxylic acid (300 mg, 52%; Yield) as dark yellow solid; MS (ES) m/z 427.2 (M+1H)⁺.

### Step 3. Preparation of methyl 1-(3-methoxy-4-{6-[3-methyl-4-({[(1R)-1-phenylethoxy]carbonyl}amino)-1,2-oxazol-5-yl]-2-azaspiro[3.3]heptan-2-yl}phenyl)cyclopropane-1-carboxylate

To a solution of 5-(2-{2-methoxy-4-[1-(methoxycarbonyl)cyclopropyl]phenyl}-2-azaspiro[3.3]heptan-6-yl)-3-methyl-1,2-oxazole-4-carboxylic acid (0.3 g, 703 umol) in toluene (10 mL, 3.26 mmol) were added, triethylamine (196 μL, 2 eq., 1.41 mmol), (1R)-1-phenylethan-1-ol (258 μL, 3 eq., 2.11 mmol) stirred reaction mixture at 50 °C. Then added {[azido(phenoxy)phosphoryl]oxy}benzene (303 μL, 2 eq., 1.41 mmol). The mixture was stirred at 85 °C for 3 h. Reaction was progressed by TLC, rm was diluted with water (20 mL) extracted with ethyl acetate (2×20 mL). The combined organic layer was washed with water (2×20 mL), brine solution (10 mL), dried over anhydrous sodium sulphate, filtered and evaporated under reduced pressure to get crude product. The crude product was purified by combiflash MPLC using ethyl acetate in n-hexane (1% to 50%) to afford methyl 1-(3-methoxy-4-{6-[3-methyl-4-({[(1R)-1-phenylethoxy]carbonyl}amino)-1,2-oxazol-5-yl]-2-azaspiro[3.3]heptan-2-yl}phenyl)cyclopropane-1-carboxylate as yellowish liquid (120 mg, 31%; Yield) MS (ES) m z 546.0 (M+H)⁺.

### Step 4. Preparation of 1-(3-methoxy-4-{6-[3-methyl-4-({[(R)-1-phenylethoxy]carbonyl}amino)-1,2-oxazol-5-yl]-2-azaspiro[3.3]heptan-2-yl}phenyl)cyclopropane-1-carboxylic acid

To a stirred solution of methyl 1-(3-methoxy-4-{6-[3-methyl-4-({[(1R)-1-phenylethoxy]carbonyl}amino)-1,2-oxazol-5-yl]-2-azaspiro[3.3]heptan-2-yl}phenyl)cyclopropane-1-carboxylate (120 mg, 220 umol) in oxolane (2 mL), methanol (2 mL, 156 mmol) solution was added lithium hydrate hydroxide (36.9 mg, 4 eq., 880 umol) in water (2 mL) and the mixture was stirred at RT for 18 hours. Reaction was progressed by LCMS and TLC, rm was concentrated, diluted with water (10 mL), washed with diethyl ether (2×10 mL), PH was adjusted with 10% Aq. citric acid up to 3 to 4, white solid was precipitated, filtered on buchnel funnel, solid was washed with water (2×10 mL), pentane (2×10 mL) and dried under vacuum to get crude, crude was given for prep HPLC Mobile phase(A): 0.1% Ammonia in water, Mobile phase(B): Acetonitrile purification to afford 1-(3-methoxy-4-{6-[3-methyl-4-({[(1R)-1-phenylethoxy]carbonyl}amino)-1,2-oxazol-5-yl]-2-azaspiro[3.3]heptan-2-yl}phenyl)cyclopropane-1-carboxylic acid as off white solid (30 mg, 26%; yield) MS (ES) m/z 532.4 (M+1H)⁻. LC purity 99.62% @240 nm.

¹H NMR (400 MHz, DMSO-d₆): δ 12.02 (s, 1H), 8.86 (S, 7=1H), 7.39-7.27 (m, 5H), 6.76-6.71 (m, 2H), 6.24 (d, J=8 Hz, 1H), 5.75 (q, J=6.4 Hz, 1H), 3.84 (s, 2H), 3.71 (s, 5H), 3.48-3.40 (m, 1H), 2.46-2.32 (m, 4H), 2.07 (s, 3H), 1.52 (s, 3H), 1.41-1.34 (m, 2H), 1.05-1.03 (m, 2H).

### Example 6. Preparation of (R)-1-(4-(3-(3-methyl-4-(((1-phenylethoxy)carbonyl) amino)isoxazol-5-yl)azetidin-1-yl)phenyl)cyclopropane-1-carboxylic acid (Compound 23)

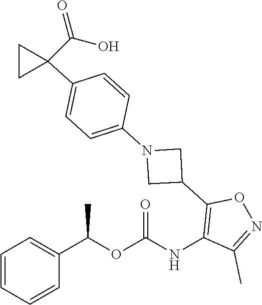 [SMILES: Cc1noc(C2CN(c3ccc(C4(C(=O)O)CC4)cc3)C2)c1NC(=O)O[C@H](C)c1ccccc1]

### Step 1. Preparation of methyl 3-(methylamino) but-2-enoate

To a mixture of methyl 3-oxobutanoate (25 g, 215.30 mmol, 23.15 mL, 1 eq) in MeOH (300 mL) was added 33% aq. MeNH₂ (31.57 g, 335.45 mmol, 1.56 eq) dropwise over 30 min at 0 °C. The mixture was stirred at 25 °C for 1 hr. The reaction solution was concentrated in vacuo at 25 °C to give methyl 3-(methylamino) but-2-enoate (27.5 g, crude) as a white solid.

### Step 2. Preparation of tert-butyl 3-chlorocarbonylazetidine-1-carboxylate

To a mixture of 1-tert-butoxycarbonylazetidine-3-carboxylic acid (10 g, 49.70 mmol, 1 eq) and DMF (182 mg, 2.48 mmol, 19 μL, 0.05 eq) in DCM (100 mL) was added oxalyl dichloride (6.31 g, 49.70 mmol, 4.35 mL, 1 eq) at 0 °C dropwise, the mixture was stirred at 25 °C for 16 hr. The mixture was concentrated to dryness in vacuo to give tert-butyl 3-chlorocarbonylazetidine-1-carboxylate (5.1 g, 23.22 mmol, 46.72% yield) as a yellow oil.

### Step 3. Preparation of tert-butyl 3-[(E)-2-methoxycarbonyl-3-(methylamino) but-2-enoyl]azetidine-1-carboxylate

To a mixture of methyl 3-(methylamino)but-2-enoate (2.5 g, 19.36 mmol, 1 eq) and pyridine (4.90 g, 61.94 mmol, 5.00 mL, 3.2 eq) in THF (30 mL) was added tert-butyl 3-chlorocarbonylazetidine-1-carboxylate (5.10 g, 23.23 mmol, 1.2 eq) drop-wise at 0 °C, the mixture was stirred at 25 °C for 16 hr. The mixture was poured into H₂O (30 mL), the resulting mixture was extracted with EtOAc (40 mL*3). The combined organic layer was dried over Na₂SO₄, filtered, and concentrated in vacuo. The residue was purified by silica gel chromatography (Petroleum ether:Ethyl acetate=10:1 to 3:1) to give tert-butyl 3-[(E)-2-methoxycarbonyl-3-(methylamino) but-2-enoyl]azetidine-1-carboxylate (1.5 g, 4.80 mmol, 24.81% yield) as a yellow oil.

### Step 4. Preparation of methyl 5-(1-tert-butoxycarbonylazetidin-3-yl)-3-methyl-isoxazole-4-carboxylate

To a mixture of tert-butyl 3-[(E)-2-methoxycarbonyl-3-(methylamino) but-2-enoyl]azetidine-1-carboxylate (1.3 g, 4.16 mmol, 1 eq) in HOAc (5 mL) was added NH₂OH·HCl (347 mg, 4.99 mmol, 1.2 eq). The mixture was stirred at 60 °C for 0.5 hr. The mixture was concentrated, the residue was dissolved in EtOAc (10 mL), treated with sat. NaHCO₃ until pH 8, and extracted with EtOAc (10 mL*3). The combined organic layer was dried over Na₂SO₄, filtered and concentrated in vacuo. The residue was purified by reversed-phase MPLC (0.1% FA condition) to give methyl 5-(1-tert-butoxycarbonylazetidin-3-yl)-3-methyl-isoxazole-4-carboxylate (585 mg, 1.97 mmol, 47.44% yield) as yellow oil.

### Step 5. Preparation of 5-(1-tert-butoxycarbonylazetidin-3-yl)-3-methyl-isoxazole-4-carboxylic acid

To a mixture of methyl 5-(1-tert-butoxycarbonylazetidin-3-yl)-3-methyl-isoxazole-4-carboxylate (585 mg, 1.97 mmol, 1 eq) in MeOH (15 mL) and H₂O (1.5 mL) was added LiOH (118 mg, 4.94 mmol, 2.5 eq). The mixture was stirred at 60 °C for 1 hr. The reaction mixture was cooled to rt, treated with HCl (1 M) until pH 6, and extracted with EtOAc (5 mL*3). The combined organic layer was dried over Na₂SO₄, filtered and concentrated in vacuo to give 5-(1-tert-butoxycarbonylazetidin-3-yl)-3-methyl-isoxazole-4-carboxylic acid (480 mg, crude) as a yellow solid.

### Step 6. Preparation of tert-butyl 3-[3-methyl-4-[[(R)-1-phenylethoxy]carbonylamino]isoxazol-5-yl]azetidine-1-carboxylate

To a mixture of 5-(1-tert-butoxycarbonylazetidin-3-yl)-3-methyl-isoxazole-4-carboxylic acid (250 mg, 886 umol, 1 eq) and (1R)-1-phenylethanol (325 mg, 2.66 mmol, 321 μL, 3 eq) in toluene (3 mL) were added DPPA (268 mg, 974 mol, 211 μL, 1.1 eq) and TEA (179 mg, 1.77 mmol, 247 μL, 2 eq). The mixture was stirred at 80 °C for 4 hr under N₂ atmosphere. The mixture was concentrated. The residue was purified by silica gel chromatography (Petroleum ether:Ethyl acetate=10:1 to 3:1) to give tert-butyl 3-[3-methyl-4-[[(1R)-1-phenylethoxy]carbonylamino]isoxazol-5-yl]azetidine-1-carboxylate (185 mg, 460.82 mol, 52.03% yield) as a colorless oil.

### Step 7. Preparation of [(1R)-1-phenylethyl]N-[5-(azetidin-3-yl)-3-methyl-isoxazol-4-yl]carbamate

To a mixture of tert-butyl 3-[3-methyl-4-[[(1R)-1-phenylethoxy]carbonylamino]isoxazol-5-yl]azetidine-1-carboxylate (150 mg, 373.64 umol, 1 eq) in i-PrOH (5 mL) was added HCl/dioxane (4 M, 1 mL). The mixture was stirred at 25 °C for 17 hr. The mixture was concentrated. The residue was purified by reversed-phase MPLC (0.1% FA condition), followed by lyophilization to give [(1R)-1-phenylethyl]N-[5-(azetidin-3-yl)-3-methyl-isoxazol-4-yl]carbamate (112 mg, 372 umol, 99.47% yield) as a colorless oil.

### Step 8. Preparation of methyl 1-[4-[3-[3-methyl-4-[[(1R)-1-phenylethoxy]carbonylamino]isoxazol-5-yl]azetidin-1-yl]phenyl]cyclopropanecarboxylate

To a mixture of [(1R)-1-phenylethyl]N-[5-(azetidin-3-yl)-3-methyl-isoxazol-4-yl]carbamate (72 mg, 239 umol, 1 eq) in DCM (5 mL) were added Cu(OAc)₂ (88 mg, 478 umol, 2 eq), TEA (48 mg, 478 umol, 67 uL, 2 eq), and [4-(1-methoxy carbonylcyclopropyl)phenyl]boronic acid (105.14 mg, 477.87 umol, 2 eq). The mixture was stirred at 20 °C for 18 hr under O₂ atmosphere. The mixture was filtered, the filtrate was concentrated. The residue was purified by prep-TLC (Petroleum ether:Ethyl acetate=3:1) to give methyl 1-[4-[3-[3-methyl-4-[[(1R)-1-phenylethoxy]carbonylamino]isoxazol-5-yl]azetidin-1-yl]phenyl]cyclopropanecarboxylate (41 mg, 86 umol, 36% yield) as a yellow oil.

### Step 9. Preparation of 1-[4-[3-[3-methyl-4-[[(1R)-1-phenylethoxy]carbonylamino]isoxazol-5-yl]azetidin-1-yl]phenyl]cyclopropanecarboxylic acid

To a mixture of methyl 1-[4-[3-[3-methyl-4-[[(1R)-1-phenylethoxy]carbonylamino]isoxazol-5-yl]azetidin-1-yl]phenyl]cyclopropanecarboxylate (41 mg, 86 umol, 1 eq) in THF (5 mL) and H₂O (1 mL) was added LiOH (6 mg, 259 umol, 3 eq). The mixture was stirred at 25 °C for 16 hr and at reflux for 32 hr. The reaction mixture was cooled to rt, treated with 1 N HCl until pH 6, and extracted with EtOAc (10 mL*3). The combined organic layer was dried over Na₂SO₄, filtered and concentrated in vacuo. The residue was purified by prep-HPLC (column: Unisil 3-100 C18 Ultra 150*50 mm*3 um; mobile phase: [water(0.225% FA)-ACN]; B %: 40%-70%, 10 min) to give 1-[4-[3-[3-methyl-4-[[(1R)-1-phenylethoxy]carbonylamino]isoxazol-5-yl]azetidin-1-yl]phenyl]cyclopropanecarboxylic acid (7.1 mg, 13.85 umol, 16.06% yield, 90% purity) as an off-white solid. [M+H]⁺=462.2. ¹H NMR (400 MHz, MeOD-d4) δ=7.49-7.26 (m, 5H), 7.17 (d, J=8.4 Hz, 2H), 6.40 (d, J=8.4 Hz, 2H), 5.81-5.67 (m, 1H), 4.15-3.97 (m, 3H), 3.96-3.85 (m, 2H), 2.13 (s, 3H), 1.59-1.46 (m, 5H), 1.15-1.08 (m, 2H).

### Example 7. Preparation of (R)-1-(4-(1-(3-methyl-4-(((1-phenylethoxy)carbonyl)amino) isoxazol-5-yl)piperidin-4-yl)phenyl)cyclopropane-1-carboxylic acid (Compound 8)

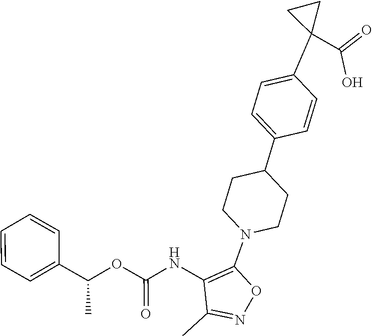 [SMILES: Cc1noc(N2CCC(c3ccc(C4(C(=O)O)CC4)cc3)CC2)c1NC(=O)O[C@H](C)c1ccccc1]

### Step 1. Preparation of tert-butyl 4-(p-tolylsufonylhydrazono)piperidine-1-carboxylate

To a mixture of 4-methylbenzenesulfonohydrazide (10.84 g, 58.22 mmol, 1 eq) in MeOH (100 mL) was added tert-butyl 4-oxopiperidine-1-carboxylate (11.6 g, 58.22 mmol, 1 eq) at 25 °C under N₂. The mixture was stirred at 25 °C for 16 hr. The mixture was filtered, the filter cake was dried in vacuo to give tert-butyl 4-(p-tolylsulfonylhydrazono) piperidine-1-carboxylate (17 g, crude) as an off-white solid.

### Step 2. Preparation of tert-butyl 4-[4-(1-ethoxycarbonylcyclopropyl)phenyl]piperidine-1-carboxylate

To a mixture of tert-butyl 4-(p-tolylsulfonylhydrazono) piperidine-1-carboxylate (5 g, 13.61 mmol, 1 eq) and in dioxane (50 mL) were added Cs₂CO₃ (13.30 g, 40.83 mmol, 3 eq) and ethyl 1-[4-(4,4,5,5-tetramethyl-1,3,2-dioxaborolan-2-yl)phenyl]cyclopropanecarboxylate (2.5 g, 7.91 mmol, 0.58 eq). The mixture was stirred at 110 °C for 3 hr. Then tert-butyl 4-(p-tolylsulfonylhydrazono) piperidine-1-carboxylate (5.00 g, 13.61 mmol, 1 eq) was added, the mixture was stirred at 110 °C for 32 hr. The mixture was poured into water (50 mL), the resulting mixture was extracted with EtOAc (50 mL*3). The combined organic was dried over Na₂SO₄, filtered and concentrated in vacuo. The residue was purified by silica gel chromatography (Petroleum ether:Ethyl acetate=20:1 to 10:1) to give tert-butyl 4-[4-(1-ethoxycarbonylcyclopropyl)phenyl]piperidine-1-carboxylate (2.3 g, 6.16 mmol, 45.25% yield) as a yellow oil.

### Step 3. Preparation of ethyl 1-[4-(4-piperidyl) phenyl]cyclopropanecarboxylate

To a mixture of tert-butyl 4-[4-(1-ethoxycarbonylcyclopropyl)phenyl]piperidine-1-carboxylate (2.3 g, 6.16 mmol, 1 eq) in DCM (9 mL) was added TFA (3 mL). The mixture was stirred at 20 °C for 3.5 hr. The mixture was concentrated to dryness in vacuo, and the residue was re-dissolved in DCM (30 mL). K₂CO₃ (10 g) was added, and the resulting mixture was stirred at 25 °C for 0.5 h before being filtered, and the filtrate was concentrated to give ethyl 1-[4-(4-piperidyl) phenyl]cyclopropanecarboxylate (1.6 g, crude) as a yellow solid.

### Step 4. Preparation of ethyl 1-[4-[1-(4-carbamoyl-3-methyl-isoxazol-5-yl)-4-piperidyl]phenyl]cyclopropanecarboxylate

A mixture of ethyl 1-[4-(4-piperidyl)phenyl]cyclopropanecarboxylate (500 mg, 1.83 mmol, 1 eq), 5-chloro-3-methyl-isoxazole-4-carboxylic acid (295.47 mg, 1.83 mmol, 1 eq) and TEA (925.40 mg, 9.15 mmol, 1.27 mL, 5 eq) in DMF (10 mL) was stirred at 40 °C for 16 hr. The mixture was cooled to rt before the addition of HATU (835 mg, 2.19 mmol, 1.2 eq). The resulting mixture was stirred at 25 °C for 10 min before being treated with NH₄Cl (196 mg, 3.66 mmol, 2 eq), and the stirring was continued at 25 °C for 1 hr. The mixture was poured into water (10 mL) and extracted with EtOAc (10 mL*3). The combined organic layer was dried over Na₂SO₄, filtered and concentrated in vacuo. The residue was triturated with EtOAc (5 mL), followed by filtration, the filter cake was dried in vacuo to give ethyl 1-[4-[1-(4-carbamoyl-3-methyl-isoxazol-5-yl)-4-piperidyl]phenyl]cyclopropanecarboxylate (260 mg, 654 mol, 35.76% yield) as a white solid.

### Step 5. Preparation of ethyl 1-[4-[1-[3-methyl-4-[[(1R)-1-phenylethoxy]carbonylanmino]isoxazol-5-yl]-4-piperidyl]phenyl]cyclopropanecarboxylate

To a mixture of ethyl 1-[4-[1-(4-carbamoyl-3-methyl-isoxazol-5-yl)-4-piperidyl]phenyl]cyclopropanecarboxylate (180 mg, 453 mol, 1 eq) in toluene (5 mL) were added bis(trifluoroacetoxy)iodo]benzene (390 mg, 905.74 mol, 2 eq) and pyridine (107 mg, 1.36 mmol, 110 μL, 3 eq). The mixture was stirred at 30 °C for 1 hr under N₂ atmosphere. Then (1R)-1-phenylethanol (277 mg, 2.26 mmol, 274 μL, 5 eq) was added, and the stirring was continued at 70 °C for 2 hr. The mixture was poured into water (10 mL), the resulting mixture was extracted with EtOAc (10 mL*3). The combined organic extracts were dried over Na₂SO₄, filtered and concentrated in vacuo. The residue was purified by silica gel chromatography (Petroleum ether:Ethyl acetate=10:1 to 3:1) to give ethyl 1-[4-[1-[3-methyl-4-[[(1R)-1-phenylethoxy]carbonylamino]isoxazol-5-yl]-4-piperidyl]phenyl]cyclopropanecarboxylate (45 mg, 86.94 mol, 19.20% yield) as a yellow oil.

### Step 6. Preparation of 1-[4-[1-[3-methyl-4-[[(1R)-1-phenylethoxy]carbonylamino]isoxazol-5-yl]-4-piperidyl]phenyl]cyclopropanecarboxylic acid

To a mixture of ethyl 1-[4-[1-[3-methyl-4-[[(1R)-1-phenylethoxy]carbonylamino]isoxazol-5-yl]-4-piperidyl]phenyl]cyclopropanecarboxylate (45 mg, 87 mol, 1 eq) in THF (5 mL) and H₂O (1 mL) was added LiOH (6 mg, 261 umol, 3 eq). The mixture was stirred at 65 °C for 64 hr. HCl aqueous solution (1 M, 3 mL) was added into the mixture, the mixture was extracted with EtOAc (10 mL*3). The combined organic was dried over Na₂SO₄, filtered and concentrated in vacuo. The residue was purified by prep-HPLC (column: Unisil 3-100 C18 Ultra 150*50 mm*3 um; mobile phase: [water (0.225% FA)-ACN]; B %: 42%-72%, 10 min), followed by lyophilization to give 1-[4-[1-[3-methyl-4-[[(1R)-1-phenylethoxy]carbonylamino]isoxazol-5-yl]-4-piperidyl]phenyl]cyclopropanecarboxylic acid (11.5 mg, 23.26 umol, 26.75% yield, 99% purity) as an off-white solid. [M+H⁺]=490.3. ¹H NMR (400 MHz, CD₃OD) δ=7.44-7.19 (m, 7H), 7.17-7.05 (m, 2H), 5.87-5.70 (m, 1H), 4.15-3.95 (m, 2H), 3.10-2.90 (m, 2H), 2.77-2.56 (m, 1H), 2.03 (s, 3H), 1.81-1.41 (m, 9H), 1.20-1.05 (m, 2H).

### Example 8. Preparation of 1-(4-{6-[3-methyl-4-({[(1R)-1-phenylethoxy]carbonyl}amino)-1,2-oxazol-5-yl]-2-azaspiro[3.3]heptan-2-yl}phenyl)cyclopropane-1-carboxylic acid (Compound 54)

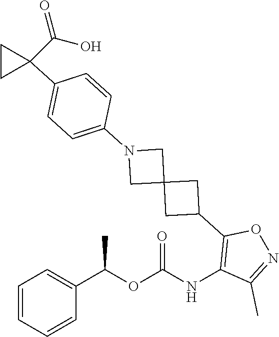 [SMILES: Cc1noc(C2CC3(C2)CN(c2ccc(C4(C(=O)O)CC4)cc2)C3)c1NC(=O)O[C@H](C)c1ccccc1]

### Step 1. Preparation of tert-butyl 6-(3-ethoxy-3-oxopropanoyl)-2-azaspiro[3.3]heptane-2-carboxylate

To a stirred solution of 2-[(tert-butoxy)carbonyl]-2-azaspiro[3.3]heptane-6-carboxylic acid (2 g, 8.29 mmol) in Acetonitrile (50 mL) was added 2-(1H-imidazole-2-carbonyl)-1H-imidazole (2.02 g, 1.5 eq., 12.4 mmol) at rt. The reaction mixture was stirred at rt for 16 hours. To a solution potassium 3-ethoxy-3-oxopropanoate (3.1 g, 2.2 eq., 18.2 mmol) in Acetonitrile (50 mL) were added triethylamine (3.49 mL, 3 eq., 24.9 mmol) and magnesium(2+) dichloride (1.74 g, 2.2 eq., 18.2 mmol), N,N-dimethylpyridin-4-amine (203 mg, 0.2 eq., 1.66 mmol) at 0 °C and stirred at rt for 8 hours, after that above solution (CDI mixture solution) was added to reaction mixture at 0 °C and stirred at 70 °C for 6 hours. Reaction was progressed by TLC & LCMS. Reaction mixture was quenched with 1 mol/L hydrochloric acid (50 mL) under ice cooling, extracted with ethyl acetate (2×150 mL). The combined organic layer was washed with water (2×100 mL), sodium bicarbonate solution (50 mL) and brine solution (50 mL), dried over anhydrous sodium sulphate, filtered and evaporated under reduced pressure to get crude product. The crude product was purified by combiflash MPLC using ethyl acetate in n-hexane (1% to 20%) to afford tert-butyl 6-(3-ethoxy-3-oxopropanoyl)-2-azaspiro[3.3]heptane-2-carboxylate as viscous liquid (2.4 g, 93% yield); MS (ES) m/z 256.1 (M+1H)⁺, −56 De-BOC mass was observed.

### Step 2. Preparation of (Z)-N-hydroxyethanecarbonimidoyl chloride

To a solution of (E)-N-ethylidenehydroxylamine (3 g, 50.8 mmol) in N,N-dimethylformamide (60.0 mL) was added 1-chloropyrrolidine-2,5-dione (7.46 g, 1.1 eq., 55.9 mmol) at 0 °C and the resulting mixture was stirred at room temperature for 3 hr. The reaction mass was diluted with water (80 mL) and extracted with diethyl ether (2×100 mL), combined organic layer was washed with brine solution dried over sodium sulfate and concentrated to afford 2-methylbut-2-ene. (Yield: 2.8 g, 59%).

### Step 3. Preparation of tert-butyl 4-[4-(ethoxycarbonyl)-3-methyl-1,2-oxazol-5-yl]piperidine-1-carboxylate

To a solution of tert-butyl 6-(3-ethoxy-3-oxopropanoyl)-2-azaspiro[3.3]heptane-2-carboxylate (1 g, 3.21 mmol) in ethanol (20 mL) and THF (20 mL) was added Sodium ethoxide (21% solution in ethanol) 21% w/w 21% v/v (6.08 mL, 5 eq., 16.1 mmol) was added at 0 °C, maintained for 30 minutes, then added (Z)-N-hydroxyethanecarbonimidoyl chloride (2.4 g, 8 eq., 25.7 mmol) and the resulting mixture was stirred at room temperature for 16 hr. Progress of the reaction was monitored by TLC and LCMS. After completion of reaction, reaction mass was diluted with water (80 mL) extracted with ethyl acetate (3×50 mL). The combined organic layer was washed with water (2×50 mL), brine solution (50 mL), dried over anhydrous sodium sulphate, filtered and evaporated to get crude. The crude product was purified by combiflash MPLC using ethyl acetate in n-hexane (1% to 10%) to afford tert-butyl 6-[4-(ethoxycarbonyl)-3-methyl-1,2-oxazol-5-yl]-2-azaspiro[3.3]heptane-2-carboxylate as yellowish liquid (Yield; 550 mg, 49%) MS (ES) m % z 351 (M+H)⁺. but m/z 295.1 (−56 De-Boc mass is observed).

### Step 4. Preparation of 5-{2-[(tert-butoxy)carbonyl]-2-azaspiro[3.3]heptan-6-yl}-3-methyl-1,2-oxazole-4-carboxylic acid

To a solution of tert-butyl 6-[4-(ethoxycarbonyl)-3-methyl-1,2-oxazol-5-yl]-2-azaspiro[3.3]heptane-2-carboxylate (550 mg, 1.57 mmol) in methanol (10 mL) was added sodium hydroxide (188 mg, 3 eq., 4.71 mmol) in water (6 mL) at room temperature, and stirred at the same temperature for 2 hours. Reaction was progressed by LCMS and TLC, rm was concentrated, diluted with water (20 mL), PH was adjusted with 1N HCL up to 3 to 4, white solid was precipitated, extracted with DCM (2×30 ml), washed with water (2×15 mL), and dried under vacuum to afford 5-{2-[(tert-butoxy)carbonyl]-2-azaspiro[3.3]heptan-6-yl}-3-methyl-1,2-oxazole-4-carboxylic acid as gummy liquid, (Yield: 450 mg, 89%); MS (ES) m/z 321.2 (M−H)⁻.

### Step 5. Preparation of 5-{2-azaspiro[3.3]heptan-6-yl}-3-methyl-1,2-oxazole-4-carboxylic acid hydrochloride

To a stirred solution of 5-{2-[(tert-butoxy)carbonyl]-2-azaspiro[3.3]heptan-6-yl}-3-methyl-1,2-oxazole-4-carboxylic acid (410 mg, 1.27 mmol) in dichloromethane (20 mL) were added Dioxane in HCL (4 mL, 8 eq., 10.2 mmol) at 0 °C, and stirred reaction mixture at room temperature for 12 hours. Progress of the reaction was monitored by TLC and LCMS. After completion of reaction, reaction mass was concentrated, washed with pentane, dried under vacuum to obtain 5-{2-azaspiro[3.3]heptan-6-yl}-3-methyl-1,2-oxazole-4-carboxylic acid hydrochloride (Yield: 275 mg, 84%) as white solid, MS (ES) m/z 221.1 (M+H)⁺.

### Step 6. Preparation of 5-(2-{4-[1-(methoxycarbonyl)cyclopropyl]phenyl}-2-azaspiro[3.3]heptan-6-yl)-3-methyl-1,2-oxazole-4-carboxylic acid

To a stirred solution of 5-{2-azaspiro[3.3]heptan-6-yl}-3-methyl-1,2-oxazole-4-carboxylic acid hydrochloride (275 mg, 1.06 mmol) in dimethylformamide (15 mL, 194 mmol) were added Cesium carbonate (1.39 g, 4 eq., 4.25 mmol), methyl 1-(4-bromophenyl)cyclopropane-1-carboxylate (325 mg, 1.2 eq., 1.28 mmol) and X-phos (50.7 mg, 0.1 eq., 106 umol), purged under argon gas for 15 minutes. Finally added Pd₂(dba)₃ (48.7 mg, 0.05 eq., 53.1 umol) and heated reaction mixture to 100 °C for 16 hours inside sealed tube. Progress of the reaction was monitored by TLC and LCMS. After completion of reaction, reaction mass was cooled to room temperature and filtered through celite, and concentrated then diluted with water (20 mL) and washed with ethyl acetate, then Aq. layer was acidified with 10% citric acid until pH is acidic, then extracted with ethyl acetate (2×100 mL), washed with water (50 mL), brine solution (50 mL), dried over anhydrous sodium sulphate, filtered and evaporated under reduced pressure to afford 5-(2-{4-[1-(methoxycarbonyl)cyclopropyl]phenyl}-2-azaspiro[3.3]heptan-6-yl)-3-methyl-1,2-oxazole-4-carboxylic acid (Yield: 325 mg, 77%) as dark yellow solid; MS (ES) m/z 397.2 (M+1H)⁺.

### Step 7. Preparation of methyl 1-(4-{6-[3-methyl-4-({[(NR)-1-phenylethoxy]carbonyl}amino)-1,2-oxazol-5-yl]-2-azaspiro[3.3]heptan-2-yl}phenyl)cyclopropane-1-carboxylate

{[azido(phenoxy)phosphoryl]oxy}benzene (163 μL, 2 eq., 757 μmol) and triethylamine (105 μL, 2 eq., 757 μmol) were added to a solution of 5-(2-{4-[1-(methoxycarbonyl)cyclopropyl]phenyl}-2-azaspiro[3.3]heptan-6-yl)-3-methyl-1,2-oxazole-4-carboxylic acid (150 mg, 378 umol) at 50 °C. The mixture was stirred at 50 °C for 30 minutes. then added (1R)-1-phenylethan-1-ol (139 mg, 3 eq., 1.14 mmol). The mixture was stirred at 80 °C for 3 h. Reaction was progressed by TLC, rm was diluted with water (20 mL) extracted with ethyl acetate (2×50 mL). The combined organic layer was washed with water (2×20 mL), brine solution (20 mL), dried over anhydrous sodium sulphate, filtered and evaporated under reduced pressure to get crude product. The crude product was purified by combiflash MPLC using ethyl acetate in n-hexane (1% to 50%) to afford methyl 1-(4-{6-[3-methyl-4-({[(1R)-1-phenylethoxy]carbonyl}amino)-1,2-oxazol-5-yl]-2-azaspiro[3.3]heptan-2-yl}phenyl)cyclopropane-1-carboxylate (30 mg, 58.2 μmol) as pale yellow solid (30 mg, 16% yield); MS (ES) m/z 516.3 (M+H)⁺.

### Step 8. Preparation of 1-(4-{6-[3-methyl-4-({[(1R)-1-phenylethoxy]carbonyl}amino)-1,2-oxazol-5-yl]-2-azaspiro[3.3]heptan-2-yl}phenyl)cyclopropane-1-carboxylic acid

To a solution of methyl 1-(4-{6-[3-methyl-4-({[(1R)-1-phenylethoxy]carbonyl}amino)-1,2-oxazol-5-yl]-2-azaspiro[3.3]heptan-2-yl}phenyl)cyclopropane-1-carboxylate (32 mg, 62.1 umol) in oxolane (4.00 mL), methanol (4.00 mL) was added lithium(1+) hydrate hydroxide (13 mg, 5 eq., 310 umol) in water (4.00 mL) at room temperature and stirred at the same temperature for 18 hours. Reaction was progressed by LCMS and TLC, rm was concentrated, diluted with water (20 mL), PH was adjusted with 10% Aq. citric acid up to 3 to 4, white solid was precipitated, filtered on Buchner funnel, solid was washed with water (2×25 mL), pentane (2×25 mL) and dried under vacuum to afford 1-(4-{6-[3-methyl-4-({[(1R)-1-phenylethoxy]carbonyl}amino)-1,2-oxazol-5-yl]-2-azaspiro[3.3]heptan-2-yl}phenyl)cyclopropane-1-carboxylic acid as white solid. (Yield: 15 mg, 48%) MS (ES) m/z 502.4 (M+H)⁺.

¹H NMR (400 MHz, DMSO-d₆): δ 12.1 (s, 1H), 8.87 (s, 1H), 7.37-7.28 (m, 5H), 7.08 (d, J 5=8.4 Hz, 2H, 6.30 (d, J=8.4 Hz, 2H), 5.72 (t, J=6.8 Hz, 1H), 3.81 (s, 2H), 3.67 (s, 2H), 3.47-3.29 (m, 1H), 2.48-2.37 (m, 4H), 2.02 (s, 3H), 1.49 (s, 3H), 1.35 (s, 2H), 1.00 (s, 2H).

### Example 9. Preparation of 1-(4-((2S,4S)-2-methyl-4-(3-methyl-4-((((R)-1-phenylethoxy)carbonyl)amino)isoxazol-5-yl)piperidin-1-yl)phenyl)cyclopropane-1-carboxylic acid (Compound 10) & 1-(4-((2R,4R)-2-methyl-4-(3-methyl-4-((((R)-1-phenylethoxy) carbonyl)amino)isoxazol-5-yl)piperidin-1-yl)phenyl)cyclopropane-1-carboxylic acid (Compound 21)

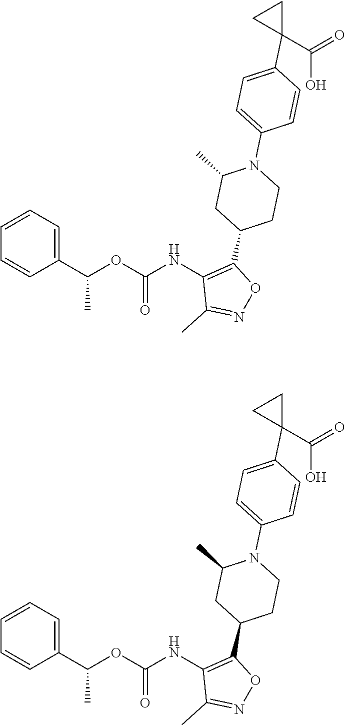 [SMILES: Cc1noc([C@H]2CCN(c3ccc(C4(C(=O)O)CC4)cc3)[C@@H](C)C2)c1NC(=O)O[C@H](C)c1ccccc1 ; Cc1noc([C@@H]2CCN(c3ccc(C4(C(=O)O)CC4)cc3)[C@H](C)C2)c1NC(=O)O[C@H](C)c1ccccc1]

### Step 1. Preparation of 1-(tert-butyl) 4-methyl 2-methylpiperidine-1,4-dicarboxylate

To a mixture of methyl 2-methylpiperidine-4-carboxylate (6.5 g, 33.56 mmol, 1 eq, HCl) in DCM (100 mL) were added TEA (10.19 g, 100.69 mmol, 14.01 mL, 3 eq) and Boc₂O (10.99 g, 50.34 mmol, 11.57 mL, 1.5 eq), the mixture was stirred at 25 °C for 1 hr. The mixture was concentrated in vacuo. The residue was purified by flash silica gel chromatography (ISCO®; 80 g SepaFlash® Silica Flash Column, Eluent of 0~10% Ethyl acetate/Petroleum ether) to give 1-(tert-butyl) 4-methyl 2-methylpiperidine-1,4-dicarboxylate (6.9 g, 26.81 mmol, 79.89% yield) as a yellow oil.

### Step 2. Preparation of 1-tert-butoxycarbonyl-2-methyl-piperidine-4-carboxylic acid

To a mixture of 01-tert-butyl 04-methyl 2-methylpiperidine-1,4-dicarboxylate (6.8 g, 26.43 mmol, 1 eq) in THF (70 mL) and H₂O (35 mL) was added LiOH (2.53 g, 105.70 mmol, 4 eq), the mixture was stirred at 25 °C for 16 hr. The mixture was concentrated in vacuo to remove THF, diluted with EtOAc (40 mL) and H₂O (40 mL), treated with HCl (1 M) aqueous solution until pH 5, and extracted with EtOAc (40 mL*3). The combined organic layer was dried over Na₂SO₄, filtered, and concentrated in vacuo to give 1-tert-butoxycarbonyl-2-methyl-piperidine-4-carboxylic acid (5.75 g, 23.63 mmol, 89.43% yield) as a yellow solid.

### Step 3. Preparation of tert-butyl 4-chlorocarbonyl-2-methyl-piperidine-1-carboxylate

To a mixture of 1-tert-butoxycarbonyl-2-methyl-piperidine-4-carboxylic acid (2 g, 8.22 mmol, 1 eq) and DMF (60.00 mg, 820.86 umol, 63.16 μL, 0.1 eq) in DCM (40 mL) was added (COCl)₂ (1.57 g, 12.33 mmol, 1.08 mL, 1.5 eq) drop-wise at 0 °C, and the resulting mixture was stirred at 25 °C for 2 hr. The mixture was concentrated to give tert-butyl 4-chlorocarbonyl-2-methyl-piperidine-1-carboxylate (4 g, crude) as a yellow oil.

### Step 4. Preparation of tert-butyl 4-[(E)-2-methoxycarbonyl-3-(methylamino) but-2-enoyl]-2-methyl-piperidine-1-carboxylate

To a mixture of methyl (Z)-3-(methylamino)but-2-enoate (1.18 g, 9.17 mmol, 1.2 eq) and pyridine (1.81 g, 22.92 mmol, 1.85 mL, 3 eq) in THF (20 mL) was added tert-butyl 4-chlorocarbonyl-2-methyl-piperidine-1-carboxylate (2 g, 7.64 mmol, 1 eq) drop-wise, and the resulting mixture was stirred at 25 °C for 17 hr. The mixture was poured into water (40 mL) and extracted with EtOAc (40 mL*3). The combined organic layer was dried over Na₂SO₄, filtered and concentrated in vacuo. The residue was purified by flash silica gel chromatography (ISCO®; 12 g SepaFlash® Silica Flash Column, Eluent of 0~25% Ethyl acetate/Petroleum ether) to give tert-butyl 4-[(E)-2-methoxycarbonyl-3-(methylamino) but-2-enoyl]-2-methyl-piperidine-1-carboxylate (2 g, 5.64 mmol, 36.92% yield) as a yellow oil.

### Step 5. Preparation of methyl 5-(1-tert-butoxycarbonyl-2-methyl-4-piperidyl)-3-methyl-isoxazole-4-carboxylate

To a mixture of tert-butyl 4-[(E)-2-methoxycarbonyl-3-(methylamino) but-2-enoyl]-2-methyl-piperidine-1-carboxylate (2 g, 5.64 mmol, 1 eq) in MeOH (20 mL) was added aq.hydroxylamine (1.12 g, 16.93 mmol, 50% purity, 3 eq). The mixture was stirred at 60 °C for 2 hr. The mixture was concentrated. The residue was purified by flash silica gel chromatography (ISCO®; 12 g SepaFlash® Silica Flash Column, Eluent of 0~10% Ethyl acetate/Petroleum ether) to give methyl 5-(1-tert-butoxycarbonyl-2-methyl-4-piperidyl)-3-methyl-isoxazole-4-carboxylate (760 mg, 2.25 mmol, 39.80% yield) as a yellow oil.

### Step 6. Preparation of methyl 3-methyl-5-(2-methyl-4-piperidyl) isoxazole-4-carboxylate HCl salt

To a mixture of methyl 5-(1-tert-butoxycarbonyl-2-methyl-4-piperidyl)-3-methyl-isoxazole-4-carboxylate (710 mg, 2.10 mmol, 1 eq) in dioxane (5 mL) was added HCl/dioxane (4 M, 5 mL), and the resulting mixture was stirred at 25 °C for 1 hr. The mixture was concentrated to give methyl 3-methyl-5-(2-methyl-4-piperidyl)isoxazole-4-carboxylate (520 mg, 1.89 mmol, 90.21% yield, HCl) as a white solid.

### Step 7. Preparation of methyl 5-[1-[4-(1-ethoxycarbonylcyclopropyl) phenyl]-2-methyl-4-piperidyl]-3-methyl-isoxazole-4-carboxylate

To a mixture of methyl 3-methyl-5-(2-methyl-4-piperidyl) isoxazole-4-carboxylate (500 mg, 1.82 mmol, 1 eq, HCl) in dioxane (10 mL) were added Cs₂CO₃ (1.78 g, 5.46 mmol, 3 eq), RuPhos Pd G₃ (152.21 mg, 181.99 mol, 0.1 eq) and ethyl 1-(4-bromophenyl)cyclopropanecarboxylate (979.58 mg, 3.64 mmol, 2 eq) under N₂, the mixture was stirred at 110 °C for 16 hr. The mixture was cooled to rt and concentrated. The residue was purified by flash silica gel chromatography (ISCO®; 40 g SepaFlash® Silica Flash Column, Eluent of 0~20% Ethyl acetate/Petroleum ether) to give methyl 5-[1-[4-(1-ethoxycarbonylcyclopropyl)phenyl]-2-methyl-4-piperidyl]-3-methyl-isoxazole-4-carboxylate (420 mg, 984.75 mol, 54.11% yield) as a yellow oil.

### Step 8. Preparation of 5-[1-[4-(1-ethoxycarbonylcyclopropyl) phenyl]-2-methyl-4-piperidyl]-3-methyl-isoxazole-4-carboxylic acid

To a mixture of methyl 5-[1-[4-(1-ethoxycarbonylcyclopropyl) phenyl]-2-methyl-4-piperidyl]-3-methyl-isoxazole-4-carboxylate (350 mg, 820.62 mol, 1 eq) in THF (5 mL) and H₂O (2.5 mL) was added LiOH (39.31 mg, 1.64 mmol, 2 eq), the mixture was stirred at 25 °C for 17 hr. HCl aqueous solution (1M) was added to adjust pH to 4, the resulting mixture was extracted with EtOAc (10 mL*3). The combined organic layer was dried over Na₂SO₄, filtered and concentrated in vacuo to give 5-[1-[4-(1-ethoxycarbonylcyclopropyl) phenyl]-2-methyl-4-piperidyl]-3-methyl-isoxazole-4-carboxylic acid (270 mg, crude) as a yellow solid.

### Step 9. Preparation of 1-[4-[4-[4-(1-hydroxy-4-phenyl-butyl)-3-methyl-isoxazol-5-yl]-1-piperidyl]phenyl]cyclopropanecarboxylic acid

To a mixture of 5-[1-[4-(1-ethoxycarbonylcyclopropyl) phenyl]-2-methyl-4-piperidyl]-3-methyl-isoxazole-4-carboxylic acid (250.00 mg, 606.09 umol, 1 eq) and (1R)-1-phenylethanol (222.13 mg, 1.82 mmol, 219.93 μL, 3 eq) in toluene (4 mL) were added DPPA (250.20 mg, 909.14 mol, 197.00 μL, 1.5 eq) and TEA (122.66 mg, 1.21 mmol, 168.72 μL, 2 eq), and the mixture was stirred at 80 °C for 1 hr. The reaction mixture was cooled to rt and concentrated in vacuo. The residue was purified by flash silica gel chromatography (ISCO®; 4 g SepaFlash® Silica Flash Column, Eluent of 0~30% Ethyl acetate/Petroleum ether) to give ethyl 1-[4-[2-methyl-4-[3-methyl-4-[[(1R)-1-phenylethoxy]carbonylamino]isoxazol-5-yl]-1-piperidyl]phenyl]cyclopropanecarboxylate (120 mg, 225.72 μmol, 37.24% yield) as a yellow oil.

### Step 10. Preparation of 1-[4-[(2S,4S)-2-methyl-4-[3-methyl-4-[[(1R)-1-phenylethoxy]carbonylamino]isoxazol-5-yl]-1-piperidyl]phenyl]cyclopropanecarboxylic acid and 1-[4-[(2R,4R)-2-methyl-4-[3-methyl-4-[[(1R)-1-phenylethoxy]carbonylamino]isoxazol-5-yl]-1-piperidyl]phenyl]cyclopropanecarboxylic acid

To a mixture of ethyl 1-[4-[2-methyl-4-[3-methyl-4-[[(1R)-1-phenylethoxy]carbonylamino]isoxazol-5-yl]-1-piperidyl]phenyl]cyclopropanecarboxylate (110 mg, 206.91 mol, 1 eq) in THF (2 mL), EtOH (1 mL) and H₂O (1 mL) was added LiOH (24.78 mg, 1.03 mmol, 5 eq). The mixture was stirred at 60 °C for 3 hr. HCl aqueous solution (1M) was added to adjust pH to 5, the resulting mixture was extracted with EtOAc (10 mL*3). The combined organic layer was dried over Na₂SO₄, filtered and concentrated in vacuo. The residue was purified by prep-HPLC (column: Unisil 3-100 C18 Ultra 150*50 mm*3 um; mobile phase: [water (FA)-ACN], B %: 12%-42%, 10 min) to give the off-white solid which was further separated by SFC (column: DAICEL CHIRALCEL OJ (250 mm*30 mm, 10 um); mobile phase: [0.1% NH₃H₂O in MEOH]; B %: 30%-30%, 3.5 min) to successively give 1-[4-[(2S,4S)-2-methyl-4-[3-methyl-4-[[(1R)-1-phenylethoxy]carbonylamino]isoxazol-5-yl]-1-piperidyl]phenyl]cyclopropanecarboxylic acid (12.56 mg, 24.94 umol, 12.05% yield, 100% purity) and 1-[4-[(2R,4R)-2-methyl-4-[3-methyl-4-[[(1R)-1-phenylethoxy]carbonylamino]isoxazol-5-yl]-1-piperidyl]phenyl]cyclopropanecarboxylic acid (13.1 mg, 26.01 umol, 12.57% yield, 100% purity), both off-white solids. The absolute configurations of the two chiral centers on the piperidine ring were unknown, and the relative configuration was cis.

1-[4-[(2S,4S)-2-methyl-4-[3-methyl-4-[[(1R)-1-phenylethoxy]carbonylamino]isoxazol-5-yl]-1-piperidyl]phenyl]cyclopropanecarboxylic acid (Compound 10): ¹H NMR (400 MHz, MeOD-d₄) δ=7.48-7.26 (m, 7H), 7.14 (d, J=8.3 Hz, 2H), 5.84-5.73 (m, 1H), 3.23-3.10 (m, 1H), 3.06-2.90 (m, 2H), 2.85-2.75 (m, 1H), 2.11 (s, 3H), 2.05-1.87 (m, 3H), 1.74-1.63 (m, 1H), 1.61-1.51 (m, 5H), 1.21-1.12 (m, 2H), 0.89 (d, J=6.1 Hz, 3H). [M+H]⁺=504.2.

1-[4-[(2R,4R)-2-methyl-4-[3-methyl-4-[[(1R)-1-phenylethoxy]carbonylamino]isoxazol-5-yl]-1-piperidyl]phenyl]cyclopropanecarboxylic acid (Compound 21): ¹H NMR (400 MHz, MeOD-d₄) δ=7.50-7.22 (m, 7H), 7.14 (d, J=8.3 Hz, 2H), 5.86-5.68 (m, 1H), 3.21-3.10 (m, 1H), 3.06-2.90 (m, 2H), 2.85-2.75 (m, 1H), 2.11 (s, 3H), 2.03-1.86 (m, 3H), 1.77-1.63 (m, 1H), 1.62-1.52 (m, 5H), 1.18-1.13 (m, 2H), 0.90 (d, J=5.7 Hz, 3H). [M+H⁺]=504.2.

### Example 10. Preparation of 1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl)cyclopropane-1-carboxylic acid (Compound 33)

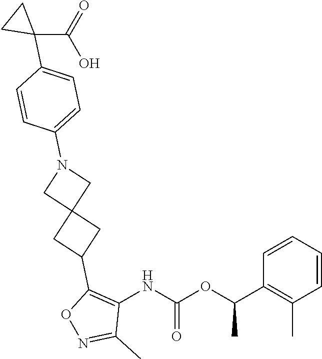 [SMILES: Cc1ccccc1[C@@H](C)OC(=O)Nc1c(C)noc1C1CC2(C1)CN(c1ccc(C3(C(=O)O)CC3)cc1)C2]

### Step 1. Preparation of methyl 1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl)cyclopropane-1-carboxylate

To a solution of 5-(2-{4-[1-(methoxycarbonyl)cyclopropyl]phenyl}-2-azaspiro[3.3]heptan-6-yl)-3-methyl-1,2-oxazole-4-carboxylic acid (0.3 g, 757 umol) in toluene (15 mL) were added triethylamine (211 μL, 2 eq., 1.51 mmol), (1R)-1-(2-methylphenyl)ethan-1-ol (309 mg, 3 eq., 2.27 mmol) stirred reaction mixture at 50 °C. Then added {[azido(phenoxy)phosphoryl]oxy}benzene (196 μL, 1.2 eq., 908 umol). The mixture was stirred at 85 °C for 3 h. Reaction was progressed by TLC, rm was diluted with water (20 mL) extracted with ethyl acetate (2×50 mL). The combined organic layer was washed with water (2×20 mL), brine solution (20 mL), dried over anhydrous sodium sulphate, filtered and evaporated under reduced pressure to get crude product. The crude product was purified by combiflash MPLC using ethyl acetate in n-hexane (1% to 50%) to afford methyl 1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl)cyclopropane-1-carboxylate as yellowish liquid (Yield; 280 mg, 70%) MS (ES) m z 530.3 (M+H)⁺.

### Step 2. Preparation of 1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl)cyclopropane-1-carboxylic acid

To a solution of methyl 1-(4-{6-[3-methyl-4-({[(1R)-1-(2-methylphenyl) ethoxy]carbonyl}amino)-1,2-oxazol-5-yl]-2-azaspiro[3.3]heptan-2-yl}phenyl)cyclopropane-1-carboxylate (280 mg, 529 umol) in oxolane (4.00 mL), methanol (4.00 mL) was added lithium(1+) hydrate hydroxide (111 mg, 5 eq., 2.64 mmol) in water (4.00 mL) at room temperature and stirred at the same temperature for 18 hours. Reaction was progressed by LCMS and TLC, reaction mas was concentrated, diluted with water (20 mL), PH was adjusted with 10% Aq. citric acid up to 3 to 4, white solid was precipitated, filtered on Buchner funnel, solid was washed with water (2×25 mL), pentane (2×25 mL) and dried under vacuum to afford 1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl)cyclopropane-1-carboxylic acid (Yield: 40 mg, 15%) as white solid. (Yield: 15 mg, 15%) MS (ES) m/z 516.3 (M+H)⁺.

¹H NMR (400 MHz, DMSO-d₆): δ 12.01 (s, 1H), 8.87 (s, 1H), 7.39 (d, J=6.4 Hz, 1H), 7.21-7.15 (m, 3H), 7.06 (d, J=8.4 Hz, 2H), 6.29 (d, J=8.8 Hz, 2H), 5.88-5.83 (m, 1H), 3.80 (s, 2H), 3.65 (s, 2H), 3.44-3.31 (m, 1H), 2.48-2.38 (m, 4H), 2.35 (s, 3H), 2.05 (s, 3H), 1.47 (d, J=5.6 Hz, 3H), 1.32 (d, J=2.8 Hz, 2H), 0.96 (d, J=1.6 Hz, 2H).

### Example 11. Preparation of 1-(4-{4-[5-chloro-3-({[(1R)-1-phenylethoxy]carbonyl}amino)thiophen-2-yl]piperidin-1-yl}phenyl)cyclopropane-1-carboxylic acid (Compound 92)

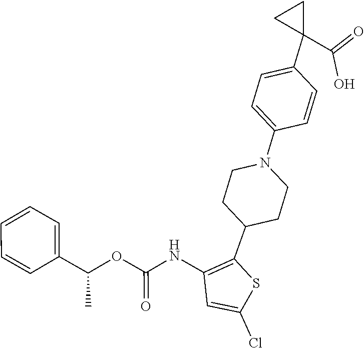 [SMILES: C[C@@H](OC(=O)Nc1cc(Cl)sc1C1CCN(c2ccc(C3(C(=O)O)CC3)cc2)CC1)c1ccccc1]

### Step 1. Preparation of tert-butyl 4-(5-chloro-3-(ethoxycarbonyl) thiophen-2-yl) piperidine-1-carboxylate

To a stirred solution of tert-butyl 4-[3-(ethoxycarbonyl) thiophen-2-yl]piperidine-1-carboxylate (0.2 g, 0.29 mmol) in DMF (2 mL) was added NCS (177 mg, 1.18 mmol) at rt in sealed tube. The reaction mixture was stirred at rt for 3 hours. The progress of the reaction was monitored by TLC & LCMS. After completion of the reaction, the reaction mixture was quenched with ice (20 mL), extracted with ethyl acetate (2×30 mL). The combined organic layer was washed with water (2×20 mL), brine solution (20 mL), dried over anhydrous sodium sulphate, filtered and evaporated under reduced pressure to get crude product. The crude product was purified by combiflash MPLC using ethyl acetate in n-hexane (1% to 20%) to afford tert-butyl 4-[5-chloro-3-(ethoxycarbonyl)thiophen-2-yl]piperidine-1-carboxylate as a viscous liquid (125 mg, 57% yield); MS (ES) m/z 274.1 (M+1H)+ (−100 mass, boc cleavage mass was observed in LCMS). ¹H NMR (400 MHz, CDCl₃): δ 7.20 (s, 1H), 4.28 (q, J=7.2 Hz, 2H), 4.14-4.11 (m, 2H), 3.93-3.87 (m, 1H), 2.81-2.61 (m, 2H), 1.50-1.47 (m, 2H), 1.49 (s, 9H), 1.35 (t, J=7.0 Hz, 3H).

### Step 2. Preparation of 2-(1-(tert-butoxycarbonyl)piperidin-4-yl)-5-chlorothiophene-3-carboxylic acid

To a solution of tert-butyl 4-[5-chloro-3-(ethoxycarbonyl)thiophen-2-yl]piperidine-1-carboxylate (200 mg, 0.535 mmol) in methanol (20 mL) was added sodium hydroxide (64.2 mg, 1.6 mmol) in water (5 mL) at room temperature and stirred at the same temperature for 18 hours. Progress of the reaction was monitored by LCMS and TLC. After completion of the reaction, it was concentrated, diluted with water (50 mL), pH was adjusted with 10% Aq. citric acid up to 3 to 4, white solid was precipitated, filtered on buchnel funnel, solid was washed with water (2×15 mL), pentane (2×20 mL) and dried under vacuum to afford 2-{1-[(tert-butoxy)carbonyl]piperidin-4-yl}-5-chlorothiophene-3-carboxylic acid as a pale yellow solid (160 mg, 86% yield); MS (ES) m/z 246.1 (M+H)⁺. (−100 mass was observed by LCMS).

### Step 3. Preparation of 5-chloro-2-(piperidin-4-yl)thiophene-3-carboxylic acid hydrochloride

To a solution of 2-{1-[(tert-butoxy)carbonyl]piperidin-4-yl}-5-chlorothiophene-3-carboxylic acid (0.18 g, 0.52 mmol) in Dichloromethane (20 mL) was added 4 M HCl in Dioxane (2.6 mL, 10.4 mmol) at 0 °C and the resulting mixture was stirred at room temperature for 8 hr. The progress of the reaction was monitored by TLC. After completion of the reaction, the reaction mixture was concentrated to afford 2-(piperidin-4-yl) thiophene-3-carboxylic acid hydrochloride as a pale yellow solid (147 mg, crude). MS (ES) m/z 245.8 (M+1H)⁺. (Free amine mass).

### Step 4. Preparation of 5-chloro-2-(1-(4-(1-(methoxycarbonyl) cyclopropyl)phenyl) piperidin-4-yl)thiophene-3-carboxylic acid

To a stirred solution of 5-chloro-2-(piperidin-4-yl)thiophene-3-carboxylic acid hydrochloride (90 mg, 0.31 mmol) and methyl 1-(4-bromophenyl) cyclopropane-1-carboxylate (89.5 mg, 0.35 mmol) in DMF (8 mL) under N₂ atm, was added Cs₂CO₃ (312 mg, 0.95 mmol) then purged with N₂ gas 3 mins, then added dicyclohexyl[2′,4′,6′-tris(propan-2-yl)-[1,1′-biphenyl]-2-yl]phosphane (15.2 mg, 0.032 mmol), tris((1E,4E)-1,5-diphenylpenta-1,4-dien-3-one) dipalladium (14.6 mg, 0.016 mmol) and the mixture was stirred at 100 °C for 8 hours in a sealed tube. The progress of the reaction was monitored by TLC. After completion of the reaction, it was cooled to rt and filtered through celite, and concentrated then diluted with water and washed with diethyl ether (2×25 mL), then aq. layer was acidified with citric acid pH-5 then extracted with ethyl acetate (2×50 mL), washed with water (25 mL), brine solution (20 mL), dried over anhydrous sodium sulphate, filtered and evaporated under reduced pressure to afford 5-chloro-2-(1-{4-[1-(methoxycarbonyl)cyclopropyl]phenyl}piperidin-4-yl)thiophene-3-carboxylic acid as a pale brown gum (130 mg, crude); MS (ES) m/z 420.1 (M+H)⁺.

### Step 5. Preparation of methyl (R)-1-(4-(4-(5-chloro-3-(((1-phenylethoxy) carbonyl)amino)thiophen-2-yl)piperidin-1-yl)phenyl)cyclopropane-1-carboxylate

To a solution of diphenylphosphinoyl azide (0.123 mL, 0.572 mmol) and TEA (0.08 mL, 0.572 mmol) were added to a solution of 5-chloro-2-(1-{4-[1-(methoxycarbonyl) cyclopropyl]phenyl}piperidin-4-yl)thiophene-3-carboxylic acid (200 mg, 0.476 mmol) in Toluene (15 mL) at 50 °C. The mixture was stirred at 50 °C for 30 minutes, then added (1R)-1-phenylethan-1-ol (0.235 mL, 1.91 mmol). The mixture was stirred at 90 °C for 8 h. The progress of the reaction was monitored by TLC. After completion of the reaction, it was diluted with water (30 mL) extracted with ethyl acetate (2×75 mL). The combined organic layer was washed with water (2×20 mL), brine solution (20 mL), dried over anhydrous sodium sulphate, filtered and evaporated under reduced pressure to get crude product. The crude product was purified by combiflash MPLC using ethyl acetate in n-hexane (1% to 20%) to afford methyl 1-(4-{4-[5-chloro-3-({[(1R)-1-phenylethoxy]carbonyl}amino) thiophen-2-yl]piperidin-1-yl}phenyl)cyclopropane-1-carboxylate as a pale yellow gum (120 mg, 46% yield); MS (ES) m/z 539.2 (M+H)⁺.

### Step 6. Preparation of (R)-1-(4-(4-(5-chloro-3-(((1-phenylethoxy)carbonyl)amino) thiophen-2-yl)piperidin-1-yl)phenyl)cyclopropane-1-carboxylic acid

To a solution of methyl 1-(4-{4-[5-chloro-3-({[(1R)-1-phenylethoxy]carbonyl}amino)thiophen-2-yl]piperidin-1-yl}phenyl)cyclopropane-1-carboxylate (100 mg, 0.185 mmol) in THF (7 mL) and methanol (7 mL) was added lithium(1+) hydrate hydroxide (31.1 mg, 0.74 mmol) in water (4 mL) at room temperature, and stirred at the same temperature for 18 hours. The progress of the reaction was monitored by LCMS and TLC. After completion of the reaction, it was concentrated, diluted with water (10 mL), pH was adjusted with 10% Aq. citric acid up to 3 to 4, then extracted with ethyl acetate (2×25 mL), washed with water (20 mL), brine solution (10 mL), dried over anhydrous sodium sulphate, filtered and evaporated under reduced pressure to get crude product, it was purified by combiflash MPLC using ethyl acetate in n-hexane (1% to 70%) to afford 1-(4-{4-[5-chloro-3-({[(1R)-1-phenylethoxy]carbonyl}amino)thiophen-2-yl]piperidin-1-yl}phenyl)cyclopropane-1-carboxylic acid as an off white solid (55 mg, 56% yield); MS (ES) m/z 525.1 (M+H)⁺. ¹H NMR (400 MHz, DMSO-d₆): δ 12.12 (s, 1H), 9.35 (s, 1H), 7.37-7.22 (m, 5H), 7.13-7.05 (m, 3H), 6.84 (d, J=8.8 Hz, 2H), 5.74 (q, J=6.8 Hz, 1H), 3.68-3.67 (m, 2H), 3.30-3.15 (m, 1H), 2.65-2.60 (m, 2H), 1.96-1.86 (m, 2H), 1.54-1.46 (m, 5H), 1.37 (d, J=4.0 Hz, 2H), 1.04 (d, J=3.6 Hz, 2H).

### Example 12. Preparation of (R)-1-(4-(4-(4-methoxy-2-(((1-phenylethoxy)carbonyl) amino)phenyl)piperidin-1-yl)phenyl)cyclopropane-1-carboxylic acid (Compound 19)

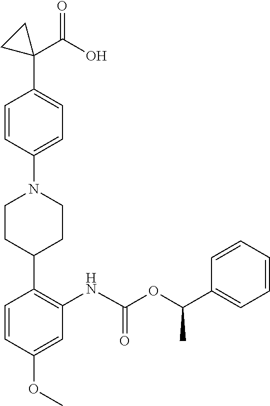 [SMILES: COc1ccc(C2CCN(c3ccc(C4(C(=O)O)CC4)cc3)CC2)c(NC(=O)O[C@H](C)c2ccccc2)c1]

### Step 1. Preparation of tert-butyl 4-(2-amino-4-methoxy-phenyl)-3,6-dihydro-2H-pyridine-1-carboxylate

To a mixture of 2-bromo-5-methoxy-aniline (1.30 g, 6.43 mmol, 1 eq) and tert-butyl 4-(4, 4,5,5-tetramethyl-1,3,2-dioxaborolan-2-yl)-3,6-dihydro-2H-pyridine-1-carboxylate (1.99 g, 6.43 mmol, 1 eq) in DMF (20 mL) were added 2 M aq. NaOH (6.43 mL, 12.86 mmol, 2 eq) and Pd(dppf)Cl₂ (471 mg, 643 umol, 0.1 eq), the mixture was stirred at 15 °C for 1 hr under N₂. H₂O (50 mL) was added, and the resulting mixture was extracted with EtOAc (50 mL*3), the combined organic phase was washed by NaCl saturated solution (30 mL*3), dried over Na₂SO₄, filtered and concentrated in vacuo. The residue was purified by flash silica gel chromatography (ISCO®; 80 g SepaFlash® Silica Flash Column, Eluent of 0~50% Ethyl acetate/Petroleum ether) to give tert-butyl 4-(2-amino-4-methoxy-phenyl)-3,6-dihydro-2H-pyridine-1-carboxylate (1.7 g, 5.59 mmol, 86.80% yield) as a yellow oil.

### Step 2. Preparation of 4-(2-amino-4-methoxy-phenyl) iperidine-1-carboxylate

To a mixture of tert-butyl 4-(2-amino-4-methoxy-phenyl)-3,6-dihydro-2H-pyridine-1-carboxylate (1.7 g, 5.59 mmol, 1 eq) in MeOH (20 mL) was added Pd/C (500 mg, 5% purity) under N₂ atmosphere. The suspension was degassed and purged with H₂ for 3 times. The mixture was stirred at 40 °C for 36 hr under H₂ (15 Psi). The mixture was filtered, the filtrate was concentrated to give 4-(2-amino-4-methoxy-phenyl) iperidine-1-carboxylate (1.41 g, crude) as a black oil.

### Step 3. Preparation of tert-butyl 4-[4-methoxy-2-(phenoxycarbonylamino) phenyl]piperidine-1-carboxylate

To a mixture of tert-butyl 4-(2-amino-4-methoxy-phenyl) iperidine-1-carboxylate (1.4 g, 4.57 mmol, 1 eq) in DCM (20 mL) were added pyridine (723 mg, 9.14 mmol, 737.60 uL, 2 eq) and phenyl carbonochloridate (1.07 g, 6.85 mmol, 859 μL, 1.5 eq), the mixture was stirred at 15 °C for 1 hr. The mixture was poured into water (10 mL) and extracted with EtOAc (40 mL*3). The combined organic layer was dried over Na₂SO₄, filtered and concentrated in vacuo. The residue was purified by flash silica gel chromatography (ISCO®; 40 g SepaFlash® Silica Flash Column, Eluent of 0~20% Ethyl acetate/Petroleum ether) to give tert-butyl 4-[4-methoxy-2-(phenoxycarbonylamino)phenyl]piperidine-1-carboxylate (1.6 g, 3.75 mmol, 82.10% yield) as a yellow oil.

### Step 4. Preparation of tert-butyl 4-[4-methoxy-2-[[(1R)-1-phenylethoxy]carbonylamino]phenyl]piperidine-1-carboxylate

To a mixture of tert-butyl 4-[4-methoxy-2-(phenoxycarbonylamino)phenyl]piperidine-1-carboxylate (1.33 g, 3.11 mmol, 1 eq) in DMF (5 mL) was added NaH (187 mg, 4.67 mmol, 60% purity, 1.5 eq) batch-wise, the mixture was stirred at 0 °C for 0.5 hr, then (1R)-1-phenylethanol (0.38 g, 3.11 mmol, 376 μL, 1 eq) was added drop-wise at 15 °C, the mixture was stirred at 15 °C for 0.5 hr. The mixture was poured into NH₄Cl saturated solution (40 mL) and extracted with EtOAc (40 mL*3). The combined organic layer was dried over Na₂SO₄, filtered and concentrated in vacuo. The residue was purified by flash silica gel chromatography (ISCO®; 40 g SepaFlash® Silica Flash Column, Eluent of 0-25% Ethyl acetate/Petroleum ether) to afford tert-butyl 4-[4-methoxy-2-[[(1R)-1-phenylethoxy]carbonylamino]phenyl]piperidine-1-carboxylate (780 mg, 1.72 mmol, 55.17% yield) as a pink oil.

### Step 5. Preparation of [(1R)-1-phenylethyl]N-[5-methoxy-2-(4-piperidyl) phenyl]carbamate

To a mixture of tert-butyl 4-[4-methoxy-2-[[(1R)-1-phenylethoxy]carbonylamino]phenyl]piperidine-1-carboxylate (780 mg, 1.72 mmol, 1 eq) in i-PrOH (5 mL) was added HCl/dioxane (4 M, 1 mL), the mixture was stirred at 15 °C for 16 hr. The mixture was poured into NaHCO₃ saturated solution (20 mL), and extracted with EtOAc (40 mL*3). The combined organic layer was dried over Na₂SO₄, filtered and concentrated in vacuo to give [(1R)-1-phenylethyl]N-[5-methoxy-2-(4-piperidyl) phenyl]carbamate (524 mg, crude) as a yellow solid.

### Step 6. Preparation of ethyl]-[4-[4-[4-methoxy-2-[[(1R)-1-phenylethoxy]carbonylamino]phenyl]-1-piperidyl]phenyl]cyclopropanecarboxylate

To a mixture of [(1R)-1-phenylethyl]N-[5-methoxy-2-(4-piperidyl)phenyl]carbamate (470 mg, 1.33 mmol, 1 eq) and [4-(1-ethoxycarbonylcyclopropyl)phenyl]boronic acid (621 mg, 2.65 mmol, 2 eq) in DCM (10 mL) were added TEA (269 mg, 2.65 mmol, 369 μL, 2 eq) and Cu(OAc)₂ (240.85 mg, 1.33 mmol, 1 eq), and the mixture was stirred at 40 °C for 16 hr under O₂ atmosphere. The mixture was filtered, the filtrate was concentrated. The residue was purified by reversed-phase MPLC (0.1% FA condition) to give ethyl 1-[4-[4-[4-methoxy-2-[[(1R)-1-phenylethoxy]carbonylamino]phenyl]-1-piperidyl]phenyl]cyclopropanecarboxylate (400 mg, 737.10 umol, 55.59% yield) as a yellow oil.

### Step 7. Preparation of 1-[4-[4-[4-methoxy-2-[[(1R)-1-phenylethoxy]carbonylamino]phenyl]-1-piperidyl]phenyl]cyclopropanecarboxylic acid

To a mixture of ethyl 1-[4-[4-[4-methoxy-2-[[(1R)-1-phenylethoxy]carbonylamino]phenyl]-1-piperidyl]phenyl]cyclopropanecarboxylate (400 mg, 737.10 umol, 1 eq) in THF (5 mL) and H₂O (5 mL) was added LiOH (53 mg, 2.21 mmol, 3 eq), the mixture was stirred at 70 °C for 32 hr. HCl aqueous solution (1 M) was added into the mixture to adjust the pH to 4, the resulting mixture was extracted with EtOAc (40 mL*3). The combined organic layer was dried over Na₂SO₄, filtered and concentrated in vacuo. The residue was purified by prep-HPLC (column: Waters Xbridge 150*25 mm*5 um; mobile phase: [water (10 mM NH₄HCO₃)-ACN]; B %: 27%-57%, 10 min) and prep-HPLC (column: Unisil 3-100 C18 Ultra 150*50 mm*3 um; mobile phase: [water(0.225% FA)-ACN]; B %: 35%-65%, 10 min), followed by lyophilization to give 1-[4-[4-[4-methoxy-2-[[(1R)-1-phenylethoxy]carbonyl amino]phenyl]-1-piperidyl]phenyl]cyclopropanecarboxylic acid (93.69 mg, 182.06 umol, 24.70% yield, 100% purity) as a white solid. [M+H]⁺=515.2 ¹H NMR (400 MHz, MeOD-d4) δ=7.47-7.37 (m, 2H), 7.34 (t, J=7.5 Hz, 2H), 7.27-7.16 (m, 4H), 6.95 (d, J=8.8 Hz, 2H), 6.88-6.75 (m, 2H), 5.80 (q, J=6.6 Hz, 1H), 3.74 (s, 3H), 3.73-3.61 (m, 2H), 2.83-2.65 (m, 2H), 2.64-2.52 (m, 1H), 1.84-1.69 (m, 4H), 1.60-1.51 (m, 5H), 1.19-1.11 (m, 2H).

### Example 13. Preparation of (R)-1-(4-(4-(1-methyl-5-(((1-phenylethoxy)carbonyl)amino)-1H-1,2,3-triazol-4-yl)piperidin-1-yl)phenyl)cyclopropane-1-carboxylic acid (Compound 60)

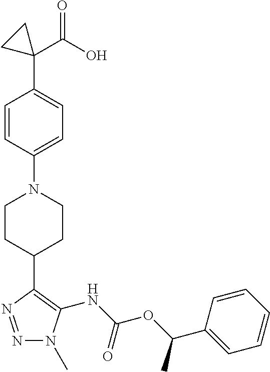 [SMILES: C[C@@H](OC(=O)Nc1c(C2CCN(c3ccc(C4(C(=O)O)CC4)cc3)CC2)nnn1C)c1ccccc1]

### Step 1. Preparation of tert-butyl 4-(2,2-dibromovinyl)piperidine-1-carboxylate

To a stirred solution of tetrabromomethane (15.5 g, 2 eq., 46.9 mmol) in dichloromethane (50 mL) was added dropwise a solution of triphenylphosphane (24.6 g, 4 eq., 93.8 mmol) in dichloromethane (50 mL) at 0 °C, and the reaction temperature maintained 20 mins, was added dropwise a solution of tert-butyl 4-formylpiperidine-1-carboxylate (5 g, 23.4 mmol) in dichloromethane (50 mL) at 0 °C, and the mixture was stirred at RT for 12 h. Progress of the reaction was monitored by TLC and LCMS. This solution was diluted with diethyl ether (200 ml), and the precipitate was filtered off. The filtrate was concentrated in vacuo to get crude. The crude product was purified by combiflash MPLC using ethyl acetate in n-hexane (1% to 10%) to afford tert-butyl 4-(2,2-dibromoethenyl)piperidine-1-carboxylate (4.5 g, 46% yield) as off white solid, MS (ES) m/z 269.0 (M+H)⁺, (M−100, boc mass cleaved in LCMS).

¹H NMR (400 MHz, CDCl₃-d): δ 6.20 (d, J=9.2 Hz, 1H), 4.05 (s, 2H), 2.76 (t, J=12.4 Hz, 2H), 2.40-2.03 (m, 1H), 1.70 (d, J=12.4 Hz, 2H), 1.60 (s, 1H), 1.44 (s, 9H), 1.30 (q, J=3.6 Hz, 2H).

### Step 2. Preparation of tert-butyl 4-(3-ethoxy-3-oxoprop-1-yn-1-yl)piperidine-1-carboxylate

To a stirred solution of tert-butyl 4-(2,2-dibromoethenyl)piperidine-1-carboxylate (5 g, 13.5 mmol) in THF (100 mL) was added butyllithium (19 mL, 2.1 eq., 28.4 mmol) at −78 °C under a nitrogen atmosphere, and the mixture was stirred at −78 °C for 1 h and at 0 °C for 1 h. ethyl carbonochloridate (4.77 mL, 3.7 eq., 50.1 mmol) in THF (50 mL) was added at −78 °C, and the reaction mixture was allowed to room temperature and continued to stir for 8 h. Progress of the reaction was monitored by TLC and LCMS. After completion of reaction, the resulting mixture was quenched ice cold water (100 mL), and extracted with Ethyl acetate (2×100 mL). The extract was washed with brine, dried and evaporated. The crude product was purified by combiflash MPLC using ethyl acetate in n-hexane (1% to 15%) to afford tert-butyl 4-(3-ethoxy-3-oxoprop-1-yn-1-yl)piperidine-1-carboxylate (288 mL, 81% yield) as pale yellow liquid; MS (ES) m/z 182.1 (M+H)⁺, (M−100, boc mass cleaved in LCMS).

¹H NMR (400 MHz, CDCl₃-d): δ 4.27-4.21 (m, 2H), 3.76-3.70 (m, 2H), 3.23-3.17 (m, 2H), 2.76-2.70 (m, 1H), 1.88-1.81 (m, 2H), 1.74-1.62 (m, 2H), 1.54 (s, 9H), 1.33-1.23 (m, 3H).

### Step 3. Preparation of tert-butyl 4-(5-(ethoxycarbonyl)-1-((trimethylsilyl)methyl)-1H-1,2,3-triazol-4-yl)piperidine-1-carboxylate

To a stirred solution of tert-butyl 4-(3-ethoxy-3-oxoprop-1-yn-1-yl)piperidine-1-carboxylate (1.4 g, 4.98 mmol) in toluene (7.98 mL, 67.5 mmol) was added (azidomethyl)trimethylsilane (2.93 mL, 4 eq., 19.9 mmol), heated to 110 °C for 8 hours. Progress of the reaction was monitored by TLC and LCMS. After completion of reaction mixture was cooled to room temperature and concentrated under reduced pressure. Crude mass was purified by normal silica gel column chromatography using ethyl acetate-hexane (10% to 15%) as eluting solvent to get two major fractions. The regio-chemistry was assigned by the 1H NOE study. One fraction is tert-butyl 4-[5-(ethoxycarbonyl)-1-[(trimethylsilyl)methyl]-1H-1,2,3-triazol-4-yl]piperidine-1-carboxylate (1.2 g, yield 58%) as light yellow liquid and other major fraction gave tert-butyl 4-[4-(ethoxycarbonyl)-1-[(trimethylsilyl)methyl]-1H-1,2,3-triazol-5-yl]piperidine-1-carboxylate (1 g, yield 48%) as white solid; LC-MS (M+H)⁺411.3 (m/e);

### Step 4. Preparation of tert-butyl 4-(5-(ethoxycarbonyl)-1-methyl-1H-1,2,3-triazol-4-yl)piperidine-1-carboxylate

To a stirred solution of tert-butyl 4-[5-(ethoxycarbonyl)-1-[(trimethylsilyl)methyl]-1H-1,2,3-triazol-4-yl]piperidine-1-carboxylate (2.2 g, 5.36 mmol) in tetrahydrofuran (34.4 mL, 422 mmol) were added water (193 mg, 2 eq., 10.7 mmol) and TBAF 1M solution in THF (1.68 g, 1.2 eq., 6.43 mmol) at 0 °C, maintained at same temperature for 1 hour. Progress of the reaction was monitored by TLC and LCMS. After completion of reaction, reaction mixture was concentrated under reduced pressure to yield crude residue. Residue was purified by combiflash MPLC using ethyl acetate in n-hexane (1% to 25%) to afford tert-butyl 4-[5-(ethoxycarbonyl)-1-methyl-1H-1,2,3-triazol-4-yl]piperidine-1-carboxylate (666 mg, 36% yield) as yellow gummy solid; MS (ES) m/z 339 (M+H)¹ but deboc mass MS (ES) m/z 283.2 (M−56) was observed in LCMS.

¹H NMR (400 MHz, CDCl₃-d): δ 4.43 (q, J=6.8 Hz, 2H), 4.29 (s, 3H), 4.26-4.24 (m, 2H), 3.34-3.36 (m, 1H), 2.88-2.82 (m, 2H), 1.92-1.79 (m, 4H), 1.54 (s, 9H), 1.40 (t, J=7.2 Hz, 2H).

### Step 5. Preparation of 4-(1-(tert-butoxycarbonyl)piperidin-4-yl)-1-methyl-1H-1,2,3-triazole-5-carboxylic acid

To a stirred solution of tert-butyl 4-[5-(ethoxycarbonyl)-1-methyl-1H-1,2,3-triazol-4-yl]piperidine-1-carboxylate (0.6 g, 1.77 mmol) in ethanol (5 mL, 171 mmol) was added sodium hydroxide (284 mg, 4 eq., 1.77 mmol) by dissolving in water (5 mL, 278 mmol) at 0 °C, maintained reaction mass at room temperature for 16 hours. Progress of the reaction was monitored by TLC and LCMS. After completion of reaction, reaction mass was concentrated to get the crude product. Obtained crude was acidified with 1 N HCl solution and stirred for 10 minutes. Product got precipitated, precipitated product was filtered, washed with dichloromethane and dried over sodium sulphate, concentrated to yield crude product 4-{1-[(tert-butoxy)carbonyl]piperidin-4-yl}-1-methyl-1H-1,2,3-triazole-5-carboxylic acid (520 mg, 94% yield) as off white solid; MS (ES) m/z 309.1 (M−1H)⁺.

### Step 6. Preparation of 1-methyl-4-(piperidin-4-yl)-1H-1,2,3-triazole-5-carboxylic acid hydrochloride

To a stirred solution of 4-{1-[(tert-butoxy)carbonyl]piperidin-4-yl}-1-methyl-1H-1,2,3-triazole-5-carboxylic acid (0.6 g, 1.93 mmol) in dichloromethane (20 mL, 312 mmol) were added 1,4-dioxane hydrochloride (1.44 g, 6 eq., 11.6 mmol) at 0 °C, and stirred reaction mixture at room temperature for 12 hours. Progress of the reaction was monitored by TLC and LCMS. After completion of reaction, reaction mass was concentrated, washed with pentane, dried under vacuum to obtain product 1-methyl-4-(piperidin-4-yl)-1H-1,2,3-triazole-5-carboxylic acid hydrochloride (450 mg, crude) as white solid; MS (ES) m/z 211.1 (M+H)⁺.

### Step 7. Preparation of 4-(1-(4-(1-(methoxycarbonyl)cyclopropyl)phenyl)piperidin-4-yl)-1-methyl-1H-1,2,3-triazole-5-carboxylic acid

To a stirred solution of 1-methyl-4-(piperidin-4-yl)-1H-1,2,3-triazole-5-carboxylic acid hydrochloride (510 mg, 2.07 mmol) in dimethylformamide (15 mL, 194 mmol) were added Cesium carbonate (2.69 g, 4 eq., 8.27 mmol), methyl 1-(4-bromophenyl)cyclopropane-1-carboxylate (633 mg, 1.2 eq., 2.48 mmol) and X-phos (98.6 mg, 0.1 eq., 207 umol), purged under argon gas for 15 minutes. Finally added Pd₂(dba)₃ (94.7 mg, 0.05 eq., 103 mol) and heated reaction mixture to 100 °C for 16 hours inside sealed tube. Progress of the reaction was monitored by TLC and LCMS. After completion of reaction, reaction mass was cooled to room temperature and filtered through celite, and concentrated then diluted with water (20 mL) and washed with ethyl acetate, then Aq. layer was acidified with 10% citric acid until pH is acidic, then extracted with ethyl acetate (2×100 mL), washed with water (50 mL), brine solution (50 mL), dried over anhydrous sodium sulphate, filtered and evaporated under reduced pressure to afford 4-(1-{4-[1-(methoxycarbonyl)cyclopropyl]phenyl}piperidin-4-yl)-1-methyl-1H-1,2,3-triazole-5-carboxylic acid (452 mg, yield 56%) as brown solid; MS (ES) m/z 385.2 (M+1H)⁺.

### Step 8. Preparation of methyl (R)-1-(4-(4-(1-methyl-5-(((1-phenylethoxy)carbonyl)amino)-1H-1,2,3-triazol-4-yl)piperidin-1-yl)phenyl)cyclopropane-1-carboxylate

To a stirred solution of 4-(1-{4-[1-(methoxycarbonyl)cyclopropyl]phenyl}piperidin-4-yl)-1-methyl-1H-1,2,3-triazole-5-carboxylic acid (0.2 g, 520 umol) in toluene (5 mL) were added triethylamine (79.5 μL, 1.1 eq., 572 mol) and (1R)-1-phenylethan-1-ol (254 mg, 4 eq., 2.08 mmol), heated reaction mixture to 50 °C, for 20 minutes. Then added DPPA (124 μL, 1.1 eq., 572 μmol), heated to 85 °C, maintained for 3 hours. Progress of the reaction was monitored by TLC and LCMS. After completion of reaction, reaction mass was diluted with water (20 mL) extracted with ethyl acetate (2×25 mL). The combined organic layer was washed with water (2×20 mL), brine solution (20 mL), dried over anhydrous sodium sulphate, filtered and evaporated under reduced pressure to get crude product. The crude product was purified by combiflash MPLC using ethyl acetate in n-hexane (41% to 70%) to afford methyl 1-(4-{4-[1-methyl-5-({[(1R)-1-phenylethoxy]carbonyl}amino)-1H-1,2,3-triazol-4-yl]piperidin-1-yl}phenyl)cyclopropane-1-carboxylate (108 mg, Yield 41%); MS (ES) m/z 504.3 (M+H)⁺.

### Step 9. Preparation of (R)-1-(4-(4-(1-methyl-5-(((1-phenylethoxy)carbonyl)amino)-1H-1,2,3-triazol-4-yl)piperidin-1-yl)phenyl)cyclopropane-1-carboxylic acid

To a stirred solution of methyl 1-(4-{4-[1-methyl-5-({[(1R)-1-phenylethoxy]carbonyl}amino)-1H-1,2,3-triazol-4-yl]piperidin-1-yl}phenyl)cyclopropane-1-carboxylate (0.1 g, 199 umol) in tetrahydrofuran (1.96 mL, 24 mmol) and methanol (1.96 mL, 48.3 mmol) was added LiOH·H2O (33.3 mg, 4 eq., 794 umol) in water (1.96 mL, 109 mmol) at 0 °C, stirred for 10 minutes. Finally maintained reaction at room temperature for 16 hours. Progress of the reaction was monitored by TLC and LCMS. After completion of reaction, reaction mass was concentrated to get the crude product. Obtained crude was acidified with 10% citric acid solution and stirred for 10 minutes. Product was precipitated, filtered and washed with pentane(10 mL) to yield product 1-(4-{4-[1-methyl-5-({[(1R)-1-phenylethoxy]carbonyl}amino)-1H-1,2,3-triazol-4-yl]piperidin-1-yl}phenyl)cyclopropane-1-carboxylic acid (55 mg, crude) as off white solid; MS (ES) m/z 430.3 (M+1H)⁺.

¹H NMR (400 MHz, DMSO-d₆): δ 7.89 (s, 1H), 7.81 (d, J=7.6 Hz, 1H), 7.51 (d, J=7.6 Hz, 1H), 7.41 (d, J=7.6 Hz, 1H), 5.15 (s, 2H), 4.05 (s, 3H), 3.60 (m, 2H), 3.31 (s, 2H), 3.11 (d, J=5.6 Hz, 2H), 2.79 (s, 3H), 2.77-2.68 (m, 1H), 2.06 (t, J=10 Hz, 2H), 1.86-1.80 (m, 2H), 1.79-1.68 (m, 2H), 1.43 (s, 2H), 0.76 (t, J=6 Hz, 3H).

### Example 14. Preparation of 1-(4-{4-[5-({[(1R)-1-(2-chlorophenyl)ethoxy]carbonyl}amino)-1-methyl-1H-1,2,3-triazol-4-yl]piperidin-1-yl}phenyl)cyclopropane-1-carboxylic acid (Compound 27)

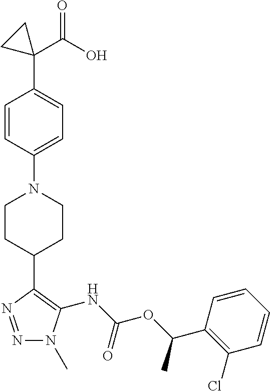 [SMILES: C[C@@H](OC(=O)Nc1c(C2CCN(c3ccc(C4(C(=O)O)CC4)cc3)CC2)nnn1C)c1ccccc1Cl]

### Step 1. Preparation of tert-butyl 4-(2,2-dibromovinyl)piperidine-1-carboxylate

To a stirred solution of tetrabromomethane (15.5 g, 2 eq., 46.9 mmol) in dichloromethane (50 mL) was added dropwise a solution of triphenylphosphane (24.6 g, 4 eq., 93.8 mmol) in dichloromethane (50 mL) at 0 °C, and the same temperature was maintained for 20 min, then dropwise addition of a solution of tert-butyl 4-formylpiperidine-1-carboxylate (5 g, 23.4 mmol) in dichloromethane (50 mL) at 0 °C, and the mixture was stirred at rt for 12 h. Progress of the reaction was monitored by TLC and LCMS. After completion of the reaction, the reaction mixture was diluted with diethyl ether (200 ml), and the precipitate was filtered off. The filtrate was concentrated in vacuo to get crude. The crude product was purified by combiflash MPLC using ethyl acetate in n-hexane (1% to 10%) to afford tert-butyl 4-(2,2-dibromoethenyl)piperidine-1-carboxylate (4.5 g, 52% yield) as an off white solid; MS (ES) m/z 269.0 (M+H)⁺, (M−100, boc mass cleaved in LCMS).

¹H NMR (400 MHz, CDCl₃): δ 6.20 (d, J=9.2 Hz, 1H), 4.05 (s, 2H), 2.76 (t, J=12.4 Hz, 2H), 2.40-2.03 (m, 1H), 1.70 (d, J=12.4 Hz, 2H), 1.60 (s, 1H), 1.44 (s, 9H), 1.30 (q, J=3.6 Hz, 2H).

### Step 2. Preparation of tert-butyl 4-(3-ethoxy-3-oxoprop-1-yn-1-yl)piperidine-1-carboxylate

To a stirred solution of tert-butyl 4-(2,2-dibromoethenyl)piperidine-1-carboxylate (5 g, 13.5 mmol) in THF (100 mL) was added n-butyl lithium 1.6M solution in hexane (19 mL, 2.1 eq., 28.4 mmol) at −78 °C under nitrogen atmosphere, and the mixture was stirred at −78 °C for 1 h and at 0 °C for 1 h. Ethylchloroformate (4.77 mL, 3.7 eq., 50.1 mmol) in THF (50 mL) was added at −78 °C, and the reaction mixture was allowed to warm up at room temperature and continued to stir for 8 h. Progress of the reaction was monitored by TLC and LCMS. After completion of reaction, the resulting mixture was quenched ice cold water (100 mL), and extracted with ethyl acetate (2×50 mL). The extract was washed with brine, dried and evaporated. The crude product was purified by combiflash MPLC using ethyl acetate in n-hexane (1% to 15%) to afford tert-butyl 4-(3-ethoxy-3-oxoprop-1-yn-1-yl)piperidine-1-carboxylate (3.2 g, 83% yield) as a yellow liquid; MS (ES) m z 182.1 (M+H)⁺, (M−100, boc mass cleaved in LCMS).

¹H NMR (400 MHz, CDCl₃): δ 4.21 (q, J=8 Hz, 2H), 3.72-3.69 (m, 2H), 3.17-3.14 (m, 2H), 2.70-2.00 (m, 1H), 1.83-1.80 (m, 2H), 1.67-1.60 (m, 2H), 1.14 (s, 9H), 1.36-1.30 (m, 3H).

### Step 3. Preparation of tert-butyl 4-(5-(ethoxycarbonyl)-1-((trimethysilyl)methyl)-1H-1,2,3-triazol-4-yl)piperidine-1-carboxylate

To a stirred solution of tert-butyl 4-(3-ethoxy-3-oxoprop-1-yn-1-yl)piperidine-1-carboxylate (0.5 g, 1.78 mmol) in toluene (2.85 mL, 24.1 mmol) was added (azidomethyl)trimethylsilane (919 mg, 4 eq., 7.11 mmol) and the reaction mixture was heated to 110 °C for 16 hours. Progress of the reaction was monitored by TLC and LCMS. After completion of reaction, the reaction mixture was cooled to room temperature and concentrated under reduced pressure. Crude mass was purified by normal silica gel column chromatography using ethyl acetate-hexane (10% to 15%) as eluting solvent to get two major fractions. The regio-chemistry was assigned by the ¹H NOE study. One fraction is tert-butyl 4-[5-(ethoxycarbonyl)-1-[(trimethylsilyl)methyl]-1H-1,2,3-triazol-4-yl]piperidine-1-carboxylate (324 mg, 44% yield) as a light yellow liquid and other major fraction gave tert-butyl 4-[4-(ethoxycarbonyl)-1-[(trimethylsilyl)methyl]-1H-1,2,3-triazol-5-yl]piperidine-1-carboxylate (254 mg, 34% yield) as a white solid; LC-MS (M+H)⁺ 411.3 (m/z).

### Step 4. Preparation of tert-butyl 4-(5-(ethoxycarbonyl)-1-methyl-1H-1,2,3-triazol-4-yl)piperidine-1-carboxylate

To a stirred solution of tert-butyl 4-[5-(ethoxycarbonyl)-1-[(trimethylsilyl)methyl]-1H-1,2,3-triazol-4-yl]piperidine-1-carboxylate (320 mg, 779 umol) in tetrahydrofuran (7 mL, 86 mmol) were added water (28.1 mg, 2 eq., 1.56 mmol) and tetrabutylazanium fluoride (245 mg, 1.2 eq., 935 umol) at 0 °C and same temperature was maintained for 1 h. Progress of the reaction was monitored by TLC and LCMS. After completion of the reaction, the reaction mixture was concentrated under reduced pressure to yield crude residue. Residue was purified by combiflash MPLC using ethyl acetate in n-hexane (1% to 25%) to afford tert-butyl 4-[5-(ethoxycarbonyl)-1-methyl-1H-1,2,3-triazol-4-yl]piperidine-1-carboxylate (120 mg, yield 45%) as a yellow gummy solid; MS (ES) m/z 339 (M+H)+ but deboc mass MS (ES) m/z 283.2 (M−56)⁺ was observed in LCMS.

¹H NMR (400 MHz, CDCl₃): δ 4.34 (d, J=7.2 Hz, 2H), 4.20 (s, 3H), 4.17-4.14 (m, 2H), 3.24-3.18 (m, 1H), 3.76 (t, J=13.2 Hz, 2H), 1.85-1.79 (m, 4H), 1.40 (s, 9H), 1.32 (d, J=4 Hz, 3H).

### Step 5. Preparation of 4-(1-(tert-butoxycarbonyl)piperidin-4-yl)-1-methyl-1H-1,2,3-triazole-5-carboxylic acid

To a stirred solution of tert-butyl 4-[5-(ethoxycarbonyl)-1-methyl-1H-1,2,3-triazol-4-yl]piperidine-1-carboxylate (120 mg, 355 umol) in THF (4 mL) was added lithium(1+) hydrate hydroxide (74.4 mg, 5 eq., 1.77 mmol) by dissolving in water (2 mL, 111 mmol) at 0 °C and the reaction mixture was stirred at room temperature for 16 hours. Progress of the reaction was monitored by TLC and LCMS. After completion of reaction, the reaction mass was concentrated to get the crude product. Obtained crude was acidified with 10% citric acid solution and stirred for 10 minutes. Product got precipitated, precipitated product was filtered, washed with water and pentane. Dried under vacuum to obtain product 4-{1-[(tert-butoxy)carbonyl]piperidin-4-yl}-1-methyl-1H-1,2,3-triazole-5-carboxylic acid (20 mg, yield 18%) as a white solid; MS (ES) m/z 309.1 (M−1H)⁺.

¹H NMR (400 MHz, CDCl₃): δ 4.37 (s, 3H), 4.27-4.22 (m, 2H), 3.42-3.34 (m, 1H), 2.91-2.85 (m, 2H), 2.03-1.93 (m, 4H), 1.50 (s, 9H).

### Step 6. Preparation of 1-methyl-4-(piperidin-4-yl)-1H-1,2,3-triazole-5-carboxylic acid hydrochloride

To a stirred solution of 4-{1-[(tert-butoxy)carbonyl]piperidin-4-yl}-1-methyl-1H-1,2,3-triazole-5-carboxylic acid (0.3 g, 967 umol) in dichloromethane (10 mL, 156 mmol) was added 1,4-dioxane hydrochloride (722 mg, 6 eq., 5.8 mmol) at 0 °C, and stirred reaction mixture at room temperature for 12 hours. Progress of the reaction was monitored by TLC and LCMS. After completion of reaction, reaction mass was concentrated, washed with pentane, dried under vacuum to obtain product 1-methyl-4-(piperidin-4-yl)-1H-1,2,3-triazole-5-carboxylic acid hydrochloride (220 mg, yield 92%) as a white solid; MS (ES) m/z 211.1 (M+H)⁺.

¹H NMR (400 MHz, CDCl₃): δ 14.08 (s, 1H), 9.02 (s, 1H), 8.76 (s, 1H), 4.19 (s, 3H), 3.57 (s, 1H), 3.47-3.40 (m, 1H), 3.36-3.32 (m, 2H), 3.06-2.97 (m, 2H), 2.33-1.96 (m, 4H).

### Step 7. Preparation of 4-(1-(4-(1-(methoxycarbonyl)cyclopropyl)phenyl)piperidin-4-yl)-1-methyl-1H-1,2,3-triazole-5-carboxylic acid

To a stirred solution of 1-methyl-4-(piperidin-4-yl)-1H-1,2,3-triazole-5-carboxylic acid hydrochloride (240 mg, 973 mol) in dimethylformamide (6 mL, 62 mmol) were added cesium carbonate (1.27 g, 4 eq., 3.89 mmol), methyl 1-(4-bromophenyl)cyclopropane-1-carboxylate (298 mg, 1.2 eq., 1.17 mmol) and X-phos (46.4 mg, 0.1 eq., 97.3 umol) and purged the mixture under argon gas for 15 minutes. Finally added Pd₂(dba)₃ (44.5 mg, 0.05 eq., 48.6 umol) and heated reaction mixture to 100 °C for 16 hours in a sealed tube. Progress of the reaction was monitored by TLC and LCMS. After completion of the reaction, reaction mass was cooled to room temperature and filtered through celite, and concentrated then diluted with water (20 mL) and washed with ethyl acetate, then Aq. layer was acidified with 10% citric acid until pH is acidic (3-4), then extracted with ethyl acetate (2×20 mL), washed with water (20 mL), brine solution (20 mL), dried over anhydrous sodium sulphate, filtered and evaporated under reduced pressure to afford 4-(1-{4-[1-(methoxycarbonyl)cyclopropyl]phenyl}piperidin-4-yl)-1-methyl-1H-1,2,3-triazole-5-carboxylic acid (232 mg, yield 62%) as a brown gummy solid; MS (ES) m/z 385.2 (M+1H)⁺.

### Step 8. Preparation of methyl 1-(4-{4-[5-({[(1R)-1-(2-chlorophenyl)ethoxy]carbonyl}amino)-1-methyl-1H-1,2,3-triazol-4-yl]piperidin-1-yl}phenyl)cyclopropane-1-carboxylate

To a stirred solution of 4-(1-{4-[1-(methoxycarbonyl)cyclopropyl]phenyl}piperidin-4-yl)-1-methyl-1H-1,2,3-triazole-5-carboxylic acid (230 mg, 598 umol) in toluene (6 mL, 3.26 mmol) were added, TEA (167 μL, 2 eq., 1.2 mmol) and (1R)-1-(2-chlorophenyl)ethan-1-ol (281 mg, 3 eq., 1.79 mmol) heated reaction mixture to 50 °C for 20 minutes. Then added {[azido(phenoxy)phosphoryl]oxy}benzene (329 mg, 2 eq., 1.2 mmol) The mixture was stirred at 85 °C for 4 hours. Progress of the reaction was monitored by TLC and LCMS. After completion of reaction, reaction mass was diluted with water (10 mL) extracted with ethyl acetate (2×20 mL). The combined organic layer was washed with water (2×10 mL), brine solution (10 mL), dried over anhydrous sodium sulphate, filtered and evaporated under reduced pressure to got crude. The crude product was taken into next step without purification to afford methyl 1-(4-{4-[5-({[(1R)-1-(2-chlorophenyl)ethoxy]carbonyl}amino)-1-methyl-1H-1,2,3-triazol-4-yl]piperidin-1-yl}phenyl)cyclopropane-1-carboxylate (480 mg) as a yellow liquid; MS (ES) m % z 538.2 (M+H)⁺.

### Step 9. Preparation of 1-(4-{4-[5-({[(1R)-1-(2-chlorophenyl)ethoxy]carbonyl}amino)-1-methyl-1H-1,2,3-triazol-4-yl]piperidin-1-yl}phenyl)cyclopropane-1-carboxylic acid

To a stirred solution of methyl 1-(4-{4-[5-({[(1R)-1-(2-chlorophenyl)ethoxy]carbonyl}amino)-1-methyl-1H-1,2,3-triazol-4-yl]piperidin-1-yl}phenyl)cyclopropane-1-carboxylate (0.5 g, 9.1 eq., 929 umol) in oxolane (3 mL), methanol (3 mL, 156 mmol) solution was added lithium(1+) hydrate hydroxide (260 mg, 61 eq., 6.2 mmol) in water (3 mL) and the mixture was stirred at rt for 18 hours. The progress of the reaction was monitored by LCMS and TLC. After completion of the reaction, the reaction mixture was concentrated, diluted with water (10 mL), washed with diethyl ether (2×10 mL), pH was adjusted with 10% Aq. citric acid up to 3 to 4, white solid was precipitated, filtered on Buchner funnel, solid was washed with water (2×10 mL), pentane (2×10 mL) and dried under vacuum to get crude, crude was given for prep HPLC Mobile phase(A): 0.1% Formic Acid in water

Mobile phase(B): Acetonitrile purification to afford 1-(4-{4-[5-({[(1R)-1-(2-chlorophenyl)ethoxy]carbonyl}amino)-1-methyl-1H-1,2,3-triazol-4-yl]piperidin-1-yl}phenyl)cyclopropane-1-carboxylic acid (15 mg, 28%) as an off white solid; MS (ES) m/z 524.0 (M+1H)⁺. LC purity 96.84%.

¹H NMR (400 MHz, DMSO-d₆): δ 12.15 (s, 1H), 9.68 (s, 1H), 7.56 (s, 1H), 7.48 (d, J=8.0 Hz, 2H), 7.38-7.22 (m, 2H), 7.14 (d, J=8.0 Hz, 2H), 6.86 (d, J=8.0 Hz, 2H), 6.02 (d, J=8.0 Hz, 1H), 3.71 (t, J=28.0 Hz, 6H), 1.79 (m, 4H), 1.55 (s, 2H), 1.38 (d, J=4.0 Hz, 2H), 1.05 (d, J=4.0 Hz, 2H).

Prep Conditions: Colum: X-Select C18 (19 mm×250 mm×5 mic) Mobile phase(A): 0.1% FA in water Mobile phase(B): Acetonitrile Flow rate: 19 ml/min

### Example 15. Preparation of (S)-1-(4-(4-(5-(((1-(2-(methoxymethyl)phenyl)ethoxy)carbonyl)amino)-1-methyl-1H-1,2,3-triazol-4-yl)piperidin-1-yl)phenyl)cyclopropane-1-carboxylic acid (Compound 24)

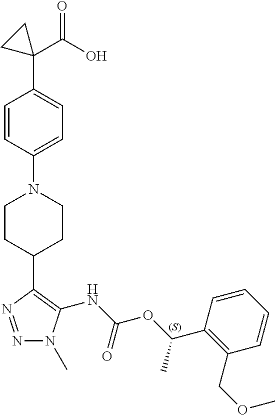

### Step 1. Preparation of 2-(methoxymethyl) benzaldehyde

To a solution of 1-bromo-2-(methoxymethyl) benzene (2 g, 9.95 mmol, 1 eq) in THF (5 mL) was added n-BuLi (2.5 M, 6 mL, 1.51 eq) at −70 °C, stirred for 1 hr, then the resulting mixture was added DMF (1.90 g, 25.99 mmol, 2.00 mL, 2.61 eq) at −70 °C and stirred for 1 hr. TLC (PE/EA=3:1, Rf=0.5) showed a new spot was detected and the 1-bromo-2-(methoxymethyl) benzene consumed. The reaction mixture was quenched by adding sat. NH₄Cl (30 mL), partitioned between EtOAc (60 mL*3) and water (80 mL), washed with brine (30 mL), dried over Na₂SO₄, filtered and evaporated under reduced pressure to give the crude product. The crude product was purified by column chromatography (SiO₂, Petroleum ether/Ethyl acetate=5/1 to 3/1) to give the 2-(methoxymethyl) benzaldehyde (1.2 g, 7.99 mmol, 80.33% yield) as a white solid. ¹H NMR (400 MHz, chloroform-d) δ=10.22 (s, 1H), 7.87 (d, J=7.5 Hz, 1H), 7.66-7.59 (m, 2H), 7.52-7.45 (m, 1H), 4.88 (s, 2H), 3.51-3.48 (m, 3H).

### Step 2. Preparation of 1-[2-(methoxymethyl) phenyl]ethanol

To a solution of 2-(methoxymethyl) benzaldehyde (1.2 g, 7.99 mmol, 1 eq) in THF (20 mL) was added MeMgBr (3 M, 5 mL, 1.88 eq) at 0 °C, then the resulting mixture stirred at 25 °C for 1 hr. TLC (PE/EA=3:1, Rf=0.3) showed a new spot was detected and 2-(methoxymethyl) benzaldehyde consumed. The reaction mixture was quenched by adding sat. NH₄Cl (30 mL), partitioned between EtOAc (120 mL) and water (50 mL), washed with brine (70 mL), dried over Na₂SO₄, filtered and evaporated under reduced pressure to give the crude product. The crude product was purified by column chromatography (SiO₂, Petroleum ether/Ethyl acetate=20/1 to 3/1) to give 1-[2-(methoxymethyl) phenyl]ethanol (0.8 g, 4.81 mmol, 60.23% yield) as the colourless oil. ¹H NMR (400 MHz, chloroform-d) δ=7.55 (d, J=7.6 Hz, 1H), 7.38 (t, J=1.8, 7.4 Hz, 1H), 7.27 (s, 2H), 5.14 (q, J=6.4 Hz, 1H), 4.67 (d, J=11.3 Hz, 1H), 4.44 (d, J=11.3 Hz, 1H), 3.41 (s, 3H), 1.55 (d, J=6.5 Hz, 3H).

### Step 3. Preparation of Ethyl1-[4-[4-[5-[1-[2-(methoxymethyl)phenyl]ethoxycarbonylamino]-1-methyl-triazol-4-yl]-1-piperidyl]phenyl]cyclopropanecarboxylate

To a solution of 5-[1-[4-(1-ethoxycarbonylcyclopropyl) phenyl]-4-piperidyl]-3-methyl-triazole-4-carboxylic acid (200 mg, 501.94 umol, 1 eq) in toluene (10 mL) was added 1-[2-(methoxymethyl) phenyl]ethanol (270 mg, 1.62 mmol, 3.24 eq), TEA (218.10 mg, 2.16 mmol, 0.3 mL, 4.29 eq), DPPA (510.00 mg, 1.85 mmol, 0.4 mL, 3.69 eq) at 0 °C, then the resulting mixture was purged with N₂ for three times and stirred at 80 °C for 12 hr. LCMS showed the desired MS was detected and the 5-[1-[4-(1-ethoxycarbonylcyclopropyl) phenyl]-4-piperidyl]-3-methyl-triazole-4-carboxylic acid consumed. TLC (PE/EA=1:1, Rf=0.4) showed a new spot was detected and the 5-[1-[4-(1-ethoxycarbonylcyclopropyl) phenyl]-4-piperidyl]-3-methyl-triazole-4-carboxylic acid consumed. The reaction mixture was partitioned between EtOAc (150 mL) and water (70 mL), washed with brine (80 mL), dried over Na₂SO₄, filtered and evaporated under reduced pressure to give the crude product. The crude product was purified by flash silica gel chromatography (ISCO; 12 g SepaFlash@ Silica Flash Column, Eluent of 0~60% Ethyl acetate/Petroleum ethergradient @40 mL/min) to give the ethyl 1-[4-[4-[5-[1-[2-(methoxymethyl) phenyl]ethoxycarbonylamino]-1-methyl-triazol-4-yl]-1-piperidyl]phenyl]cyclopropanecarboxylate (180 mg, 320.47 umol, 63.85% yield, 100% purity) as a yellow solid. MS m/z: 562.6 [M+H]+

### Step 4. Preparation of 1-[4-[4-[5-[1-[2-(methoxymethyl)phenyl]ethoxy carbonylamino]-1-methyl-triazol-4-yl]-1-piperidyl]phenyl]cyclopropanecarboxylic acid

To a solution of ethyl 1-[4-[4-[5-[1-[2-(methoxymethyl) phenyl]ethoxycarbonylamino]-1-methyl-triazol-4-yl]-1-piperidyl]phenyl]cyclopropanecarboxylate (150 mg, 267.06 mol, 1 eq) in EtOH (1 mL) was added LiOH (2 M, 1 mL, 7.49 eq), then the resulting mixture was stirred at 25 °C for 12 hr. LCMS showed the desired MS was detected and the ethyl 1-[4-[4-[5-[1-[2-(methoxymethyl)phenyl]ethoxycarbonylamino]-1-methyl-triazol-4-yl]-1-piperidyl]phenyl]cyclopropanecarboxylate consumed. The reaction mixture was acidified to pH=6 by adding AcOH. The reaction mixture was purified by prep-HPLC (Phenomenex luna Cis 150*25 mm*10 um; mobile phase: [water (FA)-ACN]; gradient: 29%-59% B over 10 min) and lyophilized to give the 1-[4-[4-[5-[1-[2-(methoxymethyl)phenyl]ethoxy carbonylamino]-1-methyl-triazol-4-yl]-1-piperidyl]phenyl]cyclopropanecarboxylic acid (90 mg, 165.29 mol, 61.89% yield, 98% purity) as a white solid. MS m/z: 534.4 [M+H]⁺

### Step 5. Preparation of J-[4-[4-[5-[[(1S)-1-[2-(methoxymethyl)phenyl]ethoxy]carbonylamino]-1-methyl-triazol-4-yl]-1-piperidyl]phenyl]cyclopropanecarboxylic acid

1-[4-[4-[5-[1-[2-(methoxymethyl)phenyl]ethoxycarbonylamino]-1-methyl-triazol-4-yl]-1-piperidyl]phenyl]cyclopropanecarboxylic acid(6) (80 mg, 149.92 umol, 1 eq) was separated by SFC (column: DAICEL CHIRALPAK IC (250 mm*30 mm, 10 um); mobile phase: [CO₂-i-PrOH (0.1% NH₃H₂O)]; B %: 50%, isocratic elution mode) to give 1-[4-[4-[5-[[(1R)-1-[2-(methoxymethyl) phenyl]ethoxy]carbonylamino]-1-methyl-triazol-4-yl]-1-piperidyl]phenyl]cyclopropanecarboxylic acid (30.3 mg, 56.78 mol, 37.88% yield, 100% purity) (Rt=1.880 min, 30.3 mg) as a white solid and 1-[4-[4-[5-[[(1S)-1-[2-(methoxymethyl) phenyl]ethoxy]carbonylamino]-1-methyl-triazol-4-yl]-1-piperidyl]phenyl]cyclopropanecarboxylic acid (41.5 mg, 74.66 mol, 49.80% yield, 96% purity)) (Rt=2.540 min, 41.5 mg) as a white solid.

¹H NMR (400 MHz, methanol-d₄) δ=7.57 (d, J=1.1 Hz, 1H), 7.41-7.34 (m, 3H), 7.23 (d, J=8.6 Hz, 2H), 6.90 (d, J=8.4 Hz, 2H), 6.12-6.04 (m, 1H), 4.80-4.75 (m, 1H), 4.40 (d, J=11.5 Hz, 1H), 3.79 (s, 3H), 3.63 (d, J=12.1 Hz, 2H), 3.36 (s, 3H), 2.70-2.60 (m, 3H), 1.90 (s, 4H), 1.58 (s, 3H), 1.48-1.44 (m, 2H), 1.02 (d, J=2.0 Hz, 2H). MS m/z: 534.3 [M+H]⁺

### Example 16. Preparation of (R)-1-(4-(4-(5-(((1-cyclohexylethoxy)carbonyl)amino)-1-methyl-1H-1,2,3-triazol-4-yl)piperidin-1-yl)phenyl)cyclopropane-1-carboxylic acid (Compound 26)

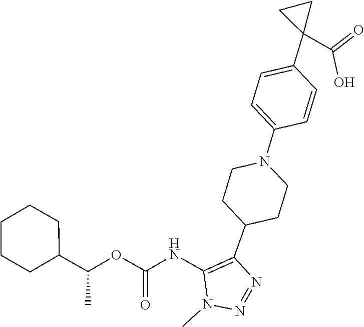 [SMILES: C[C@@H](OC(=O)Nc1c(C2CCN(c3ccc(C4(C(=O)O)CC4)cc3)CC2)nnn1C)C1CCCCC1]

### Step 1. Preparation of 1-methyl-4-(piperidin-4-yl)-1H-1,2,3-triazole-5-carboxylic acid hydrochloride

To a stirred solution of 4-{1-[(tert-butoxy)carbonyl]piperidin-4-yl}-1-methyl-1H-1,2,3-triazole-5-carboxylic acid (490 mg, 1.58 mmol) in dichloromethane (5 mL, 78.1 mmol) were added 1,4-dioxane hydrochloride (1.18 g, 6 eq., 9.47 mmol) at 0 °C, and stirred reaction mixture at room temperature for 12 hours. Progress of the reaction was monitored by TLC and LCMS. After completion of reaction, reaction mass was concentrated, washed with pentane, dried under vacuum to obtain product 1-methyl-4-(piperidin-4-yl)-1H-1,2,3-triazole-5-carboxylic acid hydrochloride (Yield: 380 mg, 98%) as white solid; MS (ES) m/z 211.1 (M+H)⁺.

### Step 2. Preparation of 4-(1-{4-[1-(methoxycarbonyl)cyclopropyl]phenyl}piperidin-4-yl)-1-methyl-1H-1,2,3-triazole-5-carboxylic acid

To a stirred solution of 1-methyl-4-(piperidin-4-yl)-1H-1,2,3-triazole-5-carboxylic acid hydrochloride (410 mg, 1.66 mmol) in dimethylformamide (10 mL, 129 mmol) were added Cesium carbonate (2.17 g, 4 eq., 6.65 mmol), methyl 1-(4-bromophenyl)cyclopropane-1-carboxylate (509 mg, 1.2 eq., 1.99 mmol) and X-phos (79.2 mg, 0.1 eq., 166 mol), purged under argon gas for 15 minutes. Finally added Pd₂(dba)₃ (76.1 mg, 0.05 eq., 83.1 umol) and heated reaction mixture to 100 °C for 16 hours inside sealed tube. Progress of the reaction was monitored by TLC and LCMS. After completion of reaction, reaction mass was cooled to room temperature and filtered through celite, and concentrated then diluted with water (20 mL) and washed with ethyl acetate, then Aq. layer was acidified with 10% citric acid until pH is acidic, then extracted with ethyl acetate (2×100 mL), washed with water (50 mL), brine solution (50 mL), dried over anhydrous sodium sulphate, filtered and evaporated under reduced pressure to afford 4-(1-{4-[1-(methoxycarbonyl)cyclopropyl]phenyl}piperidin-4-yl)-1-methyl-1H-1,2,3-triazole-5-carboxylic acid (yield: 330 mg, 52%) as brown solid; MS (ES) m/z 385.2 (M+1H)⁺.

### Step 3. Preparation of methyl 1-(4-{4-[5-({[(1R)-1-cyclohexylethoxy]carbonyl}amino)-1-methyl-1H-1,2,3-triazol-4-yl]piperidin-1-yl}phenyl)cyclopropane-1-carboxylate

To a solution of 4-(1-{4-[1-(methoxycarbonyl)cyclopropyl]phenyl}piperidin-4-yl)-1-methyl-1H-1,2,3-triazole-5-carboxylic acid (150 mg, 390 umol) in toluene (15 mL) were added triethylamine (109 μL, 2 eq., 780 umol), (1R)-1-cyclohexylethan-1-ol (150 mg, 3 eq., 1.17 mmol) stirred reaction mixture at 50 °C. Then added {[azido(phenoxy)phosphoryl]oxy}benzene (101 μL, 1.2 eq., 468 umol). The mixture was stirred at 85 °C for 3 h. Reaction was progressed by TLC, rm was diluted with water (20 mL) extracted with ethyl acetate (2×50 mL). The combined organic layer was washed with water (2×20 mL), brine solution (20 mL), dried over anhydrous sodium sulphate, filtered and evaporated under reduced pressure to get crude product. The crude product was purified by combiflash MPLC using ethyl acetate in n-hexane (1% to 50%) to afford methyl 1-(4-{4-[5-({[(1R)-1-cyclohexylethoxy]carbonyl}amino)-1-methyl-1H-1,2,3-triazol-4-yl]piperidin-1-yl}phenyl)cyclopropane-1-carboxylate as yellowish liquid (Yield; 80 mg, 40%) MS (ES) m/z 510.3 (M+H)⁺.

### Step 4. Preparation of (R)-1-(4-(4-(5-(((1-cyclohexylethoxy)carbonyl)amino)-1-methyl-1H-1,2,3-triazol-4-yl)piperidin-1-yl)phenyl)cyclopropane-1-carboxylic acid

To a stirred solution of methyl 1-(4-{4-[5-({[(1R)-1-cyclohexylethoxy]carbonyl}amino)-1-methyl-1H-1,2,3-triazol-4-yl]piperidin-1-yl}phenyl)cyclopropane-1-carboxylate (80 mg, 157 umol) in oxolane (2 mL), methanol (2 mL, 156 mmol) solution was added lithium(1+) hydrate hydroxide (26.3 mg, 4 eq., 628 umol) in water (2 mL) and the mixture was stirred at RT for 18 hours. Reaction was progressed by LCMS and TLC, rm was concentrated, diluted with water (20 mL), washed with diethyl ether (2×10 mL), PH was adjusted with 10% Aq. citric acid up to 3 to 4, white solid was precipitated, filtered on buchnel funnel, solid was washed with water (2×10 mL), pentane (2×10 mL) and dried under vacuum to afford 1-(4-{4-[5-({[(1R)-1-cyclohexylethoxy]carbonyl}amino)-1-methyl-1H-1,2,3-triazol-4-yl]piperidin-1-yl}phenyl)cyclopropane-1-carboxylic acid as off white solid (48 mg, 62% yield); MS (ES) m/z 496.4 (M+1H)⁻. LC purity 99.52% @240 nm.

¹H NMR (400 MHz, DMSO-d₆): δ 12.09 (s, 1H), 9.32 (s, 1H), 7.13 (d, J=8.4 Hz, 2H), 6.86 (d, J=8.4 Hz, 2H), 4.63 (q, J=6.4 Hz, 1H), 3.82-3.71 (m, 5H), 2.76-2.67 (m, 3H), 1.83 (d, J=3.6 Hz, 4H), 1.79-1.64 (m, 4H), 1.46-1.43 (m, 1H), 1.39-1.33 (m, 2H), 1.24-1.09 (m, 7H), 1.04 (d, J=2.8 Hz, 4H).

### Example 17. Preparation of (4-{4-[3-methyl-4-({[(1R)-1-phenylethoxy]carbonyl}amino)-1,2-oxazol-5-yl]piperidin-1-yl}phenyl)cyclopropane-1-carboxylic acid (Compound 87)

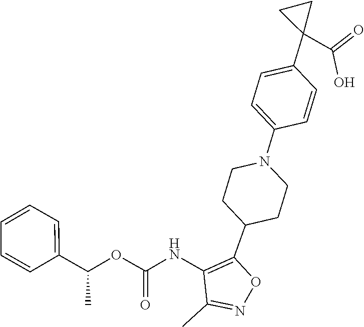 [SMILES: Cc1noc(C2CCN(c3ccc(C4(C(=O)O)CC4)cc3)CC2)c1NC(=O)O[C@H](C)c1ccccc1]

### Step 1. Preparation of methyl (R)-1-(4-(4-(3-methyl-4-(((1-phenylethoxy)carbonyl)amino)isoxazol-5-yl)piperidin-1-yl)phenyl)cyclopropane-1-carboxylate

To a stirred solution of 5-(1-{4-[1-(methoxycarbonyl)cyclopropyl]phenyl}piperidin-4-yl)-3-methyl-1,2-oxazole-4-carboxylic acid (770 mg, 1.82 mmol, Example 18, Step 1) in toluene (10 mL) were added triethylamine (278 μL, 1.1 eq., 2 mmol) and (1R)-1-phenylethan-1-ol (660 μL, 3 eq., 5.45 mmol), heated to 50 °C, maintained for 30 minutes. Then added DPPA (434 μL, 1.1 eq., 2.01 mmol) and heated reaction mixture to 85 °C, for 4 hours. Progress of the reaction was monitored by TLC and LCMS. After completion of reaction, reaction mass was diluted with water (10 mL) extracted with ethyl acetate (2×15 mL). The combined organic layer was washed with water (2×20 mL), brine solution (20 mL), dried over anhydrous sodium sulphate, filtered and evaporated under reduced pressure to get crude product. The crude product was purified by combiflash MPLC using ethyl acetate in n-hexane (1% to 28%) to afford methyl 1-(4-{4-[3-methyl-4-({[(1R)-1-phenylethoxy]carbonyl}amino)-1,2-oxazol-5-yl]piperidin-1-yl}phenyl)cyclopropane-1-carboxylate (418 mg, 45% yield); MS (ES) m/z 504.3 (M+H)⁺.

### Step 2. Preparation of 1-(4-{4-[3-methyl-4-({[(1R)-1-phenylethoxy]carbonyl}amino)-1,2-oxazol-5-yl]piperidin-1-yl}phenyl)cyclopropane-1-carboxylic acid

To a stirred solution of methyl 1-(4-{4-[3-methyl-4-({[(1R)-1-phenylethoxy]carbonyl}amino)-1,2-oxazol-5-yl]piperidin-1-yl}phenyl)cyclopropane-1-carboxylate (55 mg, 0.11 mmol) in THF (2 mL) and methanol (2 mL) was added Lithium hydroxide Monohydrate (30 mg, 6 eq., 0.7 mmol) by dissolving in water (2 mL) at 0 °C, maintained reaction mass at room temperature for 16 hours. Progress of the reaction was monitored by TLC and LCMS. After completion of reaction, reaction, reaction mass was concentrated to get the crude product. Obtained crude was acidified with 10% citric acid solution and stirred for 10 minutes. Product got precipitated, precipitated product was filtered, washed with water and pentane. Dried under vacuum to obtain product 1-(4-{4-[3-methyl-4-({[(1R)-1-phenylethoxy]carbonyl}amino)-1,2-oxazol-5-yl]piperidin-1-yl}phenyl)cyclopropane-1-carboxylic acid (48 mg, 88% yield) as off white solid; MS (ES) m/z 490.0 (M+H)+; LC purity: 99.5%

¹H NMR (400 MHz, DMSO-d₆): δ 12.10 (s, 1H), 8.90 (s, 1H), 7.37-7.45 (m, 5H), 7.12 (d, J=8.0 Hz, 2H), 6.83 (d, J=8.0 Hz, 2H), 5.74-5.69 (q, 1H), 3.65 (d, J=12.0 Hz, 2H), 2.89-2.85 (m, 2H), 2.69-2.65 (m, 2H), 2.01 (s, 3H), 1.80-1.73 (m, 4H), 1.50-1.48 (d, J=8.4 Hz, 3H), 1.37-1.36 (t, J=2.4 Hz, 2H), 1.04-1.01 (t, J=2.8 Hz, 2H);

### Example 18. Preparation of (R)-1-(4-(4-(4-(((1-(2-ethylphenyl)ethoxy)carbonyl)amino)-3-methylisoxazol-5-yl)piperidin-1-yl)phenyl)cyclopropane-1-carboxylic acid (Compound 38)

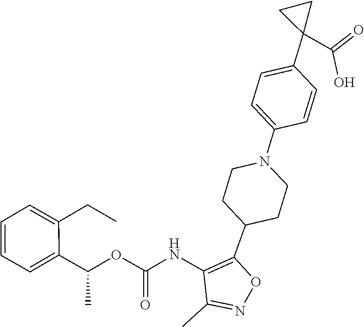 [SMILES: CCc1ccccc1[C@@H](C)OC(=O)Nc1c(C)noc1C1CCN(c2ccc(C3(C(=O)O)CC3)cc2)CC1]

### Step 1. Preparation of 5-(1-(4-(1-(methoxycarbonyl)cyclopropyl)phenyl)piperidin-4-yl)-3-methylisoxazole-4-carboxylic acid

To a stirred solution of 3-methyl-5-(piperidin-4-yl)-1,2-oxazole-4-carboxylic acid hydrochloride (2 g, 8.11 mmol) in dimethylformamide (30 mL, 387 mmol) were added Cesium carbonate (10.6 g, 4 eq., 32.4 mmol), methyl 1-(4-bromophenyl)cyclopropane-1-carboxylate (2.48 g, 1.2 eq., 9.73 mmol) and X-phos (386 mg, 0.1 eq., 811 umol), purged under argon gas for 15 minutes. Finally added Pd₂(dba)₃ (371 mg, 0.05 eq., 405 umol) and heated reaction mixture to 100 °C for 16 hours inside sealed tube. Progress of the reaction was monitored by TLC and LCMS. After completion of reaction, reaction mass was cooled to room temperature and filtered through celite, and concentrated then diluted with water (20 mL) and washed with ethyl acetate, then Aq. layer was acidified with 10% citric acid until pH is acidic, then extracted with ethyl acetate (2×100 mL), washed with water (50 mL), brine solution (50 mL), dried over anhydrous sodium sulphate, filtered and evaporated under reduced pressure to afford 5-(1-{4-[1-(methoxycarbonyl)cyclopropyl]phenyl}piperidin-4-yl)-3-methyl-1,2-oxazole-4-carboxylic acid (2.5 g, 80%) as pale yellow solid; MS (ES) in z 385.2 (M+1H)⁺.

### Step 2. Preparation of methyl (R)-1-(4-(4-(4-(((1-(2-ethylphenyl)ethoxy)carbonyl)amino)-3-methylisoxazol-5-yl)piperidin-1-yl)phenyl)cyclopropane-1-carboxylate

To a solution of 5-(1-{4-[1-(methoxycarbonyl)cyclopropyl]phenyl}piperidin-4-yl)-3-methyl-1,2-oxazole-4-carboxylic acid (150 mg, 390 mol) in toluene (10 mL, 3.26 mmol) were added, TEA(109 μL, 2 eq., 780 umol), (1R)-1-(2-ethylphenyl)ethan-1-ol (117 mg, 2 eq., 780 umol) stirred reaction mixture at 50 °C. Then added DIPEA (168 μL, 2 eq., 780 mol). The mixture was stirred at 85 °C for 3 h. Reaction was progressed by TLC, rm was diluted with water (20 mL) extracted with ethyl acetate (2×20 mL). The combined organic layer was washed with water (2×20 mL), brine solution (10 mL), dried over anhydrous sodium sulphate, filtered and evaporated under reduced pressure to get crude product. The crude product was purified by combiflash MPLC using ethyl acetate in n-hexane (18% to 30%) to afford methyl 1-(4-{4-[4-({[(1R)-1-(2-ethylphenyl)ethoxy]carbonyl}amino)-3-methyl-1,2-oxazol-5-yl]piperidin-1-yl}phenyl)cyclopropane-1-carboxylate (76 mg, 36% yield) yellowish liquid. MS (ES) m/z 532.3 (M+H)⁺.

### Step 3. Preparation of (R)-1-(4-(4-(4-(((1-(2-ethylphenyl)ethoxy)carbonyl)amino)-3-methylisoxazol-5-yl)piperidin-1-yl)phenyl)cyclopropane-1-carboxylic acid

To a stirred solution of methyl 1-(4-{4-[4-({[(1R)-1-(2-ethylphenyl)ethoxy]carbonyl}amino)-3-methyl-1,2-oxazol-5-yl]piperidin-1-yl}phenyl)cyclopropane-1-carboxylate (70 mg, 132 umol) in tetrahydrofuran (1.3 mL, 15.9 mmol) and methanol (1.3 mL, 32 mmol) was added LiOH·H2O (22.1 mg, 4 eq., 527 umol) in water (1.3 mL, 72 mmol) at 0 °C, stirred for 10 minutes. Finally maintained reaction at room temperature for 16 hours. Progress of the reaction was monitored by TLC and LCMS. After completion of reaction, reaction mass was concentrated to get the crude product. Obtained crude was acidified with 10% citric acid solution and stirred for 10 minutes. Product was precipitated. Formed product was filtered, washed with water and pentane. Then dried under vacuum to get product 1-(4-{4-[4-({[(1R)-1-(2-ethylphenyl)ethoxy]carbonyl}amino)-3-methyl-1,2-oxazol-5-yl]piperidin-1-yl}phenyl)cyclopropane-1-carboxylic acid (40 mg, 59%) as white solid; MS (ES) m/z 518.4 (M+1H)⁺. LCMS Purity 98.14%.

¹H NMR (400 MHz, DMSO-d₆): δ 12.08 (s, 1H), 8.87 (s, 1H), 7.45-7.35 (m, 4H), 7.21 (d, J=4 Hz, 2H), 7.51 (d, J=7.2 Hz, 2H), 5.96 (q, J=6.4 Hz, 1H), 3.74-3.66 (m, 2H), 2.87 (s, 1H), 2.70-2.65 (m, 4H), 2.11 (s, 3H), 1.95-1.76 (m, 4H), 1.66-1.51 (m, 3H), 1.40-1.38 (m, 2H), 1.18 (t, J=3.6 Hz, 3H), 0.93-0.84 (m, 2H).

### Example 19. Preparation of 1-(4-{4-[4-({[(1R)-1-(2-chlorophenyl)ethoxy]carbonyl}amino)-3-methyl-1,2-oxazol-5-yl]piperidin-1-yl}phenyl)cyclopropane-1-carboxylic acid (Compound 74)

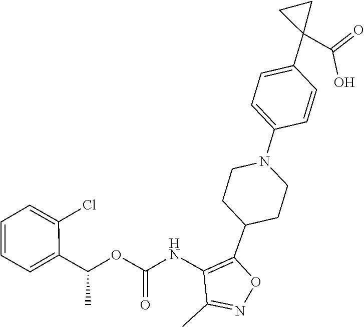 [SMILES: Cc1noc(C2CCN(c3ccc(C4(C(=O)O)CC4)cc3)CC2)c1NC(=O)O[C@H](C)c1ccccc1Cl]

### Step 1. Preparation of methyl (R)-1-(4-(4-(4-(((1-(2-chlorophenyl)ethoxy)carbonyl) amino)-3-methylisoxazol-5-yl)piperidin-1-yl)phenyl)cyclopropane-1-carboxylate

{[azido(phenoxy)phosphoryl]oxy}benzene (101 μL, 1.2 eq., 468 mol) and triethylamine (109 μL, 2 eq., 780 mol) were added to a solution of 5-(1-{4-[1-(methoxycarbonyl)cyclopropyl]phenyl}piperidin-4-yl)-3-methyl-1,2-oxazole-4-carboxylic acid (150 mg, 390 μmol) in Toluene (10 mL) at 50 °C. The mixture was stirred at 50 °C for 30 minutes, then added (1R)-1-(2-chlorophenyl)ethan-1-ol (244 mg, 4 eq., 1.56 mmol). The mixture was stirred at 90 °C for 8 h. Reaction was progressed by TLC, rm was diluted with water (20 mL) extracted with ethyl acetate (2×100 mL). The combined organic layer was washed with water (2×50 mL), brine solution (50 mL), dried over anhydrous sodium sulphate, filtered and evaporated under reduced pressure to get crude product. The crude product was purified by combiflash MPLC using ethyl acetate in n-hexane (1% to 20%) to afford methyl 1-(4-{4-[4-({[(1R)-1-(2-chlorophenyl)ethoxy]carbonyl}amino)-3-methyl-1,2-oxazol-5-yl]piperidin-1-yl}phenyl)cyclopropane-1-carboxylate as off white solid (90 mg, 42% yield); ¹H NMR (400 MHz, DMSO-d₆): δ 9.01 (s, 1H), 7.56-7.33 (m, 4H), 7.16 (d, J=8.0 Hz, 2H), 6.87 (d, J=8.0 Hz, 2H), 5.99 (q, 1H), 3.70 (m, 2H), 3.53 (s, 3H), 2.90 (m, 1H), 2.68-2.65 (m, 2H), 2.04 (s, 3H), 1.81-1.75 (m, 4H), 1.53-1.48 (m, 3H), 1.42 (t, J=2.8 Hz, 2H), 1.12 (t, J=2.8 Hz, 2H); MS (ES) m/z 539.0 (M+H)⁺; LC purity: 86%.

### Step 2. Preparation of (R)-1-(4-(4-(4-(((1-(2-chlorophenyl)ethoxy)carbonyl)amino)-3-methylisoxazol-5-yl)piperidin-1-yl)phenyl)cyclopropane-1-carboxylic acid

To a stirred solution of methyl 1-(4-{4-[4-({[(1R)-1-(2-chlorophenyl)ethoxy]carbonyl}amino)-3-methyl-1,2-oxazol-5-yl]piperidin-1-yl}phenyl)cyclopropane-1-carboxylate (70.0 mg, 130 umol) in THF (5.0 mL), MeOH (5.0 mL) was added lithium(1+) hydroxide (31.2 mg, 10 eq., 1.30 mmol) in water (5.0 mL) and the mixture was stirred at RT for 12 hours. Reaction was progressed by TLC, The reaction solution was concentrate to remove ethanol and THE, diluted with water (5 mL) and acidified with citric acid solution pH-5, and filtered and dried to get 1-(4-{4-[4-({[(1R)-1-(2-chlorophenyl)ethoxy]carbonyl}amino)-3-methyl-1,2-oxazol-5-yl]piperidin-1-yl}phenyl)cyclopropane-1-carboxylic acid as off white solid (37.0 mg, 54% yield); ¹H NMR (400 MHz, DMSO-d₆): δ 12.14 (s, 1H), 9.01 (s, 1H), 7.54-7.32 (m, 4H), 7.12 (d, J=8.0 Hz, 2H), 6.84 (d, J=8.0 Hz, 2H), 5.96 (q, 1H), 3.68-3.65 (m, 2H), 2.87 (m, 1H), 2.68-2.65 (m, 2H), 2.01 (s, 3H), 1.81-1.75 (m, 4H), 1.50-1.45 (m, 3H), 1.36 (t, J=2.8 Hz, 2H), 1.03-1.02 (t, J=2.8 Hz, 2H); MS (ES) m/z 525.0 (M+H)⁺; LC purity: 98%.

### Example 20. Preparation of (1R)-1-phenylethyl N-{3-methyl-5-[1-(4-{1-[(prop-2-ene-1-sulfonyl)carbamoyl]cyclopropyl}phenyl)piperidin-4-yl]-1,2-oxazol-4-yl}carbamate (Compound 2)

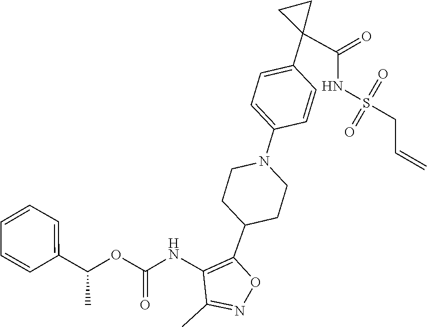 [SMILES: C=CCS(=O)(=O)NC(=O)C1(c2ccc(N3CCC(c4onc(C)c4NC(=O)O[C@H](C)c4ccccc4)CC3)cc2)CC1]

### Step 1. Preparation of methyl 1-(4-{4-[3-methyl-4-({[(1R)-1-phenylethoxy]carbonyl}amino)-1,2-oxazol-5-yl]piperidin-1-yl}phenyl)cyclopropane-1-carboxylate

To a stirred solution of 5-(1-{4-[1-(methoxycarbonyl)cyclopropyl]phenyl}piperidin-4-yl)-3-methyl-1,2-oxazole-4-carboxylic acid (1.4 g, 3.64 mmol) in toluene (20 mL) were added triethylamine (557 μL, 1.1 eq., 4.01 mmol) and (1R)-1-phenylethan-1-ol (1.32 mL, 3 eq., 10.9 mmol) and the reaction mixture was heated at 50 °C for 30 minutes. Then added DPPA (868 μL, 1.1 eq., 4.01 mmol) and the reaction mixture was heated at 85 °C for 4 hours. Progress of the reaction was monitored by TLC and LCMS. After completion of reaction, reaction mass was diluted with water (20 mL) extracted with ethyl acetate (2×25 mL). The combined organic layer was washed with water (2×20 mL) and brine solution (20 mL), dried over anhydrous sodium sulphate, filtered and evaporated under reduced pressure to get crude product. The crude product was purified by combiflash MPLC using ethyl acetate in n-hexane (1% to 28%) to afford methyl 1-(4-{4-[3-methyl-4-({[(1R)-1-phenylethoxy]carbonyl}amino)-1,2-oxazol-5-yl]piperidin-1-yl}phenyl)cyclopropane-1-carboxylate (835 mg, 45% yield) as a gummy solid; MS (ES) m/z 504.3 (M+H)⁺.

### Step 2. Preparation of 1-(4-{4-[3-methyl-4-({[(1R)-1-phenylethoxy]carbonyl}amino)-1,2-oxazol-5-yl]piperidin-1-y}phenyl)cyclopropane-1-carboxylic acid

To a stirred solution of methyl 1-(4-{4-[3-methyl-4-({[(1R)-1-phenylethoxy]carbonyl}amino)-1,2-oxazol-5-yl]piperidin-1-yl}phenyl)cyclopropane-1-carboxylate (1.1 g, 2.18 mmol) in THF (5 mL) and methanol (5 mL) was added Lithium hydroxide Monohydrate (550 mg, 6 eq., 13.1 mmol) by dissolving in water (5 mL) at 0 °C and the reaction mixture was stirred at room temperature for 16 hours. Progress of the reaction was monitored by TLC and LCMS. After completion of reaction, reaction, reaction mass was concentrated to get the crude product. Obtained crude was acidified with 10% citric acid solution and stirred for 10 minutes. Product got precipitated, precipitate was filtered, washed with water and pentane. Dried under vacuum to obtain product 1-(4-{4-[3-methyl-4-({[(1R)-1-phenylethoxy]carbonyl}amino)-1,2-oxazol-5-yl]piperidin-1-yl}phenyl)cyclopropane-1-carboxylic acid (950 mg, 88% yield) as an off white solid; MS (ES) m/z 490.2 (M+1H)⁺;

### Step 3. Preparation of (1R)-1-phenylethyl N-{3-methyl-5-[1-(4-{]-[(prop-2-ene-1-sulfonyl)carbamoyl]cyclopropyl}phenyl)piperidin-4-yl]-1,2-oxazol-4-yl}carbamate

To a stirred solution of 1-(4-{4-[3-methyl-4-({[(1R)-1-phenylethoxy]carbonyl}amino)-1,2-oxazol-5-yl]piperidin-1-yl}phenyl)cyclopropane-1-carboxylic acid (0.2 g, 409 mol) in dichloromethane (5 mL, 78.1 mmol) were added TEA (113 μL, 2 eq., 817 μmol), EDC·HCL (66.5 mg, 1.5 eq., 613 μmol) and HOBT (82.8 mg, 1.5 eq., 613 umol) at 0 °C and the reaction mixture was stirred at room temperature for 15 minutes. Finally added prop-2-ene-1-sulfonamide (74.2 mg, 1.5 eq., 613 mol) and stirred at rt for 16 hours. Progress of the reaction was monitored by TLC & LCMS. After completion of reaction, reaction mass was quenched with water and extracted with DCM (2×20 mL), washed with water (2×20 mL)) and brine solution (20 mL), dried over anhydrous sodium sulphate, filtered and evaporated under reduced pressure to get crude product. The crude product was purified by combiflash MPLC using ethyl acetate in n-hexane (75% to 80%) to afford (1R)-1-phenylethyl N-{3-methyl-5-[1-(4-{1-[(prop-2-ene-1-sulfonyl)carbamoyl]cyclopropyl}phenyl)piperidin-4-yl]-1,2-oxazol-4-yl}carbamate (148 mg, 61% yield) as a yellow gummy solid; MS (ES) m/z 593.2 (M+H)⁺@240.0 nm.

¹H NMR (400 MHz, DMSO-d₆): δ 10.81 (s, 1H), 8.91 (s, 1H), 7.39-7.29 (m, 5H), 7.15 (d, J=8.8 Hz, 2H), 6.87 (d, J=8.4 Hz, 2H), 5.76-5.69 (m, 2H), 5.38-5.28 (m, 2H), 4.09 (d, J=5.2 Hz, 2H), 3.73-3.70 (m, 2H), 2.89 (bs, 1H), 2.73-2.66 (m, 2H), 2.03 (s, 3H), 1.85-1.74 (m, 4H), 1.51 (d, J=4.8 Hz, 3H), 1.36 (bs, 2H), 1.05 (bs, 2H).

### Example 21. Preparation of 1-[({[l-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)methyl]cyclopropane-1-carboxylic acid (Compound 82)

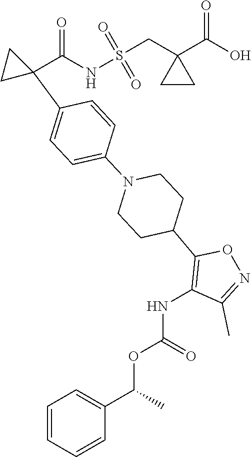 [SMILES: Cc1noc(C2CCN(c3ccc(C4(C(=O)NS(=O)(=O)CC5(C(=O)O)CC5)CC4)cc3)CC2)c1NC(=O)O[C@H](C)c1ccccc1]

### Step 1. Preparation of methyl 1-[({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)methyl]cyclopropane-1-carboxylate

To a stirred solution of 1-(4-{4-[3-methyl-4-({[(1R)-1-phenylethoxy]carbonyl}amino)-1,2-oxazol-5-yl]piperidin-1-yl}phenyl)cyclopropane-1-carboxylic acid (0.3 g, 613 umol) in tetrahydrofuran (6 mL, 73.7 mmol) were added 1-(1H-imidazole-1-carbonyl)-1H-imidazole (99.4 mg, 613 umol) and DMAP (7.49 mg, 0.1 eq., 61.3 umol) at rt and the reaction mixture was heated at 70 °C for 2 hours. Then cooled to room temperature and added methyl 1-(sulfamoylmethyl)cyclopropane-1-carboxylate (118 mg, 613 umol) and DBU (367 μL, 4 eq., 2.45 mmol) at 0 °C and stirred at rt for 16 hours. Progress of the reaction was monitored by TLC & LCMS. After completion of reaction, reaction mass was quenched with ice cold water, extracted with ethyl acetate (2×20 mL). The combined organic layer was washed with water (2×10 mL), sodium bicarbonate solution (10 mL) and brine solution (10 mL), dried over anhydrous sodium sulphate, filtered and evaporated under reduced pressure to get crude product. The crude product was purified by prep HPLC Mobile phase (A): 0.1% Ammonia in water, Mobile phase(B): Acetonitrile purification to afford methyl 1-[({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)methyl]cyclopropane-1-carboxylate (240 mg, 58%) as a white solid; MS (ES) m/z 665.6 (M+1H)⁺. LC purity 99.72%.

¹H NMR (400 MHz, DMSO-d₆): δ 10.83 (s, 1H), 8.91 (s, 1H), 7.39-7.29 (m, 5H), 7.16 (d, J=8.0 Hz, 2H), 6.89 (d, J=8.0 Hz, 2H), 5.76-5.71 (m, 1H), 3.71 (d, J=12.0 Hz, 4H), 3.58 (s, 3H), 2.89 (s, 1H), 2.73-2.66 (m, 2H), 2.03 (s, 3H), 1.85-1.71 (m, 4H), 1.51 (d, J=4.0 Hz, 3H), 1.47 (s, 2H), 1.21 (s, 2H), 1.11-1.04 (m, 4H).

### Step 2. Preparation of 1-[({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)methyl]cyclopropane-1-carboxylic acid

To a stirred solution of methyl 1-[({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)methyl]cyclopropane-1-carboxylate (0.1 g, 1.2 eq., 150 mol) in methanol (1 mL, 24.7 mmol), tetrahydrofuran (1 mL, 12.3 mmol) solution was added lithium(1+) hydrate hydroxide (41.1 mg, 8 eq., 980 μmol in water (1 mL, 55.5 mmol) and the mixture was stirred at rt for 18 hours. The progress of the reaction was monitored by LCMS and TLC. After completion of the reaction, the reaction mixture was concentrated, diluted with water (10 mL), washed with diethyl ether (2×10 mL), pH was adjusted with 10% Aq. citric acid up to 3 to 4, white solid was precipitated, filtered on Buckner funnel, solid was washed with water (2×10 mL), pentane (2×10 mL) and dried under vacuum to afford crude. The crude was purified by prep HPLC to afford 1-[({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)methyl]cyclopropane-1-carboxylic acid (45 mg, 56%) as a white solid; MS (ES) m/z 651.5 (M+1H)⁺. LC purity 97.10%.

¹H NMR (400 MHz, DMSO-d₆): δ 8.92 (s, 1H), 7.39-7.30 (m, 6H), 7.07 (d, J=12.0 Hz, 2H), 6.80 (d, J=8.0 Hz, 2H), 5.76-5.71 (m, 1H), 3.63 (d, J=12.0 Hz, 2H), 3.34 (s, 2H), 2.85 (m, 1H), 2.68-2.62 (m, 2H), 2.03 (s, 3H), 1.82-1.75 (m, 4H), 1.52 (d, J=4.0 Hz, 3H), 1.24 (t, J=8.0 Hz, 2H), 1.03-0.97 (m, 4H), 0.74-0.73 (d, J=4.0 Hz, 2H).

### Example 22. Preparation of 3-({[1-(4-{4-[4-({[(1R)-1-(2-ethylphenyl)ethoxy]carbonyl}amino)-3-methyl-1,2-oxazol-5-yl]piperidin-1-yl}phenyl)cyclopropyl]formamido}sulfonyl)propanoic acid (Compound 37)

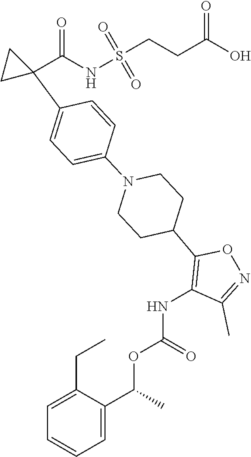 [SMILES: CCc1ccccc1[C@@H](C)OC(=O)Nc1c(C)noc1C1CCN(c2ccc(C3(C(=O)NS(=O)(=O)CCC(=O)O)CC3)cc2)CC1]

### Step 1. Preparation of methyl (R)-1-(4-(4-(4-(((1-(2-ethylphenyl)ethoxy)carbonyl)amino)-3-methylisoxazol-5-yl)piperidin-1-yl)phenyl)cyclopropane-1-carboxylate

To a solution of 5-(1-{4-[1-(methoxycarbonyl)cyclopropyl]phenyl}piperidin-4-yl)-3-methyl-1,2-oxazole-4-carboxylic acid (0.4 g, 1.04 mmol) in toluene (10 mL, 3.26 mmol) were added, TEA (290 μL, 2 eq., 2.08 mmol), (1R)-1-(2-ethylphenyl)ethan-1-ol (313 mg, 2 eq., 2.08 mmol) and the reaction mixture stirred at 50 °C for 1 h. Then added DPPA (448 μL, 2 eq., 2.08 mmol) and the mixture was stirred at 85 °C for 12 h. Progress of the reaction was monitored by TLC. After completion of the reaction, the reaction mixture was diluted with water (20 mL), extracted with ethyl acetate (2×20 mL). The combined organic layer was washed with water (2×20 mL) and brine solution (10 mL), dried over anhydrous sodium sulphate, filtered and evaporated under reduced pressure to get crude product. The crude product was purified by combiflash MPLC using ethyl acetate in n-hexane (18% to 30%) to afford methyl 1-(4-{4-[4-({[(1R)-1-(2-ethylphenyl)ethoxy]carbonyl}amino)-3-methyl-1,2-oxazol-5-yl]piperidin-1-yl}phenyl)cyclopropane-1-carboxylate (220 mg, 40% yield) as a yellowish liquid. MS (ES) m z 532.3 (M+H)⁺.

### Step 2. Preparation of 1-(4-{4-[4-({[(R)-1-(2-ethylphenyl)ethoxy]carbonyl}amino)-3-methyl-1,2-oxazol-5-yl]piperidin-1-yl}phenyl)cyclopropane-1-carboxylic acid

To a stirred solution of methyl 1-(4-{4-[4-({[(1R)-1-(2-ethylphenyl)ethoxy]carbonyl}amino)-3-methyl-1,2-oxazol-5-yl]piperidin-1-yl}phenyl)cyclopropane-1-carboxylate (270 mg, 508 mol) in tetrahydrofuran (4.91 mL, 60.3 mmol) and methanol (4.91 mL, 121 mmol) was added LiOH·H₂O (128 mg, 6 eq., 3.05 mmol) in water (4.91 mL, 272 mmol) at 0 °C and stirred for 10 minutes. Then the reaction mixture was stirred at room temperature for 16 hours. Progress of the reaction was monitored by TLC and LCMS. After completion of reaction, reaction mass was concentrated to get the crude product.

Obtained crude was acidified with 10% citric acid solution and stirred for 10 minutes. Product was precipitated. It was filtered, washed with water and pentane. Then dried under vacuum to get product 1-(4-{4-[4-({[(1R)-1-(2-ethylphenyl)ethoxy]carbonyl}amino)-3-methyl-1,2-oxazol-5-yl]piperidin-1-yl}phenyl) cyclopropane-1-carboxylic acid (190 mg, 72% yield) as a white solid; MS (ES) m/z 518.4 (M+1H)⁺.

¹H NMR (400 MHz, DMSO-d₆): δ 12.08 (s, 1H), 8.87 (s, 1H), 7.45-7.35 (m, 4H), 7.21 (d, J=4 Hz, 2H), 7.51 (d, J=7.2 Hz, 2H), 5.96 (q, J=6.4 Hz, 1H), 3.74-3.66 (m, 2H), 2.87 (s, 1H), 2.70-2.65 (m, 4H), 2.11 (s, 3H), 1.95-1.76 (m, 4H), 1.66-1.51 (m, 3H), 1.40-1.38 (m, 2H), 1.18 (t, J=3.6 Hz, 3H), 0.93-0.84 (m, 2H).

### Step 3. Preparation of methyl 3-({[1-(4-{4-[4-({[(1R)-1-(2-ethylphenyl)ethoxy]carbonyl}amino)-3-methyl-1,2-oxazol-5-yl]piperidin-1-yl}phenyl)cyclopropyl]formamido}sulfonyl) propanoate

To a stirred solution of 1-(4-{4-[4-({[(1R)-1-(2-ethylphenyl)ethoxy]carbonyl}amino)-3-methyl-1,2-oxazol-5-yl]piperidin-1-yl}phenyl)cyclopropane-1-carboxylic acid (186 mg, 359 mol) in dichloromethane (5 mL, 78.1 mmol) were added triethylamine (146 μL, 3 eq., 1.08 mmol), EDC·HCl (115 mg, 3 eq., 1.08 mmol), HOBT (97.1 mg, 2 eq., 719 umol) and DMAP (4.39 mg, 0.1 eq., 35.9 umol) at 0 °C and stirred at room temperature for 15 minutes. Finally added methyl 3-sulfamoylpropanoate (120 mg, 2 eq., 719 umol) and stirred at rt for 16 hours. Progress of the reaction was monitored by TLC & LCMS. After completion of reaction, reaction mass was quenched with ice cold water, extracted with ethyl acetate (2×50 mL). The combined organic layer was washed with water (2×25 mL), sodium bicarbonate solution (15 mL) and brine solution (15 mL), dried over anhydrous sodium sulphate, filtered and evaporated under reduced pressure to get crude product which was purified by combiflash MPLC using methanol in DCM (5% to 15%) to afford methyl 3-({[1-(4-{4-[4-({[(1R)-1-(2-ethylphenyl)ethoxy]carbonyl}amino)-3-methyl-1,2-oxazol-5-yl]piperidin-1-yl}phenyl)cyclopropyl]formamido}sulfonyl)propanoate (210 mg, 87% yield) as a gummy brown solid; MS (ES) m/z 667.1 (M+1H)⁺.

### Step 4. Preparation of 3-({[1-(4-{4-[4-({[(1R)-1-(2-ethylphenyl)ethoxy]carbonyl}amino)-3-methyl-1,2-oxazol-5-yl]piperidin-1-yl}phenyl)cyclopropyl]formamido}sulfonyl)propanoic acid

To a stirred solution of methyl 3-({[1-(4-{4-[4-({[(1R)-1-(2-ethylphenyl)ethoxy]carbonyl}amino)-3-methyl-1,2-oxazol-5-yl]piperidin-1-yl}phenyl) cyclopropyl]formamido}sulfonyl)propanoate (0.1 g, 150 umol) in tetrahydrofuran (2.5 mL, 30.7 mmol) and methanol (2.5 mL, 61.7 mmol) was added LiOH·H₂O (25.2 mg, 4 eq., 0.6 mmol) by dissolving in water (2.5 mL, 139 mmol) at 0 °C and stirred for 10 minutes. Then, the reaction mixture was stirred at room temperature for 16 hours. Progress of the reaction was monitored by TLC and LCMS. After completion of reaction, reaction mass was concentrated to get the crude product. Obtained crude was acidified with 10% citric acid solution and stirred for 10 minutes. Product was precipitated. The precipitate was filtered, washed with water and pentane dried under vacuum to yield crude product. The obtained crude was purified by preparative HPLC using method Mobile Phase (A): 0.1% Formic Acid in water and Mobile Phase (B): ACN to yield

3-({[1-(4-{4-[4-({[(1R)-1-(2-ethylphenyl)ethoxy]carbonyl}amino)-3-methyl-1,2-oxazol-5-yl]piperidin-1-yl}phenyl)cyclopropyl]formamido}sulfonyl)propanoic acid (49 mg, 50% yield) as a white solid; MS (ES) m/z 653.6 (M+1H)⁺. LCMS Purity 98.20% @254.0 nm.

¹H NMR (400 MHz, DMSO-d₆): δ 12.54 (s, 1H), 10.86 (s, 1H), 8.89 (s, 1H), 7.51 (s, 1H), 7.31-6.99 (m, 5H), 6.93-6.85 (m, 2H), 5.96 (q, J=6.4 Hz, 1H), 3.73-3.66 (m, 2H), 3.54-3.49 (m, 2H), 2.89-2.87 (m, 1H), 2.76-2.67 (m, 2H), 2.66-2.56 (m, 2H), 2.08 (s, 3H), 1.85-1.76 (m, 4H), 1.52-1.51 (m, 3H), 1.40-1.36 (m, 2H), 1.27-1.25 (m, 2H), 1.20-1.17 (m, 3H), 1.86-1.80 (m, 2H).

### Example 23. Preparation of (1R)-1-phenylethyl N-(5-{1-[4-(1-{[(1-carbamoylcyclopropyl)sulfonyl]carbamoyl}cyclopropyl)phenyl]piperidin-4-yl}-3-methyl-1,2-oxazol-4-yl)carbamate (Compound 73)

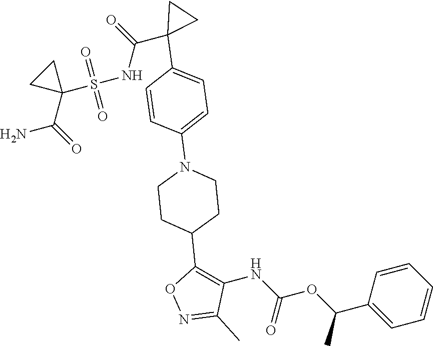 [SMILES: Cc1noc(C2CCN(c3ccc(C4(C(=O)NS(=O)(=O)C5(C(N)=O)CC5)CC4)cc3)CC2)c1NC(=O)O[C@H](C)c1ccccc1]

### Step 1. Preparation of methyl]-({[1-(4-{4-[3-methyl-4-({[(R)-1-phenylethoxy]carbonyl}amino)-1,2-oxazol-5-yl]piperidin-1-yl}phenyl)cyclopropyl]formamido}sulfonyl) cyclopropane-1-carboxylate

To a stirred solution of 1-(4-{4-[3-methyl-4-({[(1R)-1-phenylethoxy]carbonyl}amino)-1,2-oxazol-5-yl]piperidin-1-yl}phenyl)cyclopropane-1-carboxylic acid (0.3 g, 613 mol) in tetrahydrofuran (2 mL, 12.3 mmol) were added 1-(1H-imidazole-1-carbonyl)-1H-imidazole (149 mg, 1.5 eq., 919 mol) and DMAP (7.49 mg, 0.1 eq., 61.3 umol) at room temperature, heated to 70 °C for 2 hours. Then cooled to room temperature and added methyl 1-sulfamoylcyclopropane-1-carboxylate (165 mg, 1.5 eq., 919 umol) and DBU (367 μL, 4 eq., 2.45 mmol) at rt, maintained for 16 hours at RT. Progress of the reaction was monitored by TLC and LCMS. After completion of reaction, reaction mass was quenched with 1.0N HCl solution (5 mL) extracted with ethyl acetate (2×25 mL). The combined organic layer was washed with water (2×20 mL), brine solution (20 mL), dried over anhydrous sodium sulphate, filtered and evaporated under reduced pressure to get crude product. The crude product was purified by combiflash MPLC using ethyl acetate in n-hexane (68% to 70%) to afford product methyl 1-({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)cyclopropane-1-carboxylate (370 mg, 93% Yield) as colorless liquid. MS (ES) m/z 651.0 (M+1H)⁺.

### Step 2. Preparation of (1R)-1-phenylethyl N-(5-{1-[4-(1-{[(1-carbamoylcyclopropyl) sulfonyl]carbamoyl}cyclopropyl)phenyl]piperidin-4-yl}-3-methyl-1,2-oxazol-4-yl)carbamate

To a stirred solution of methyl 1-({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)cyclopropane-1-carboxylate (0.1 g, 154 μmol) in THF (5 mL) was added Ammonia (15 μL, 4 eq., 615 umol) and the reaction mixture was stirred 60 °C for 16 h. Progress of the reaction was monitored by TLC and LCMS. After completion of reaction, the resulting mixture was concentrated to get crude product. crude was given for prep HPLC Mobile phase(A): 0.1% Ammonia in water, Mobile phase(B): Acetonitrile purification to afford (1R)-1-phenylethyl N-(5-{1-[4-(1-{[(1-carbamoylcyclopropyl)sulfonyl]carbamoyl}cyclopropyl)phenyl]piperidin-4-yl}-3-methyl-1,2-oxazol-4-yl)carbamate as off white solid (10 mg, 10%: Yield) MS (ES) m/z 636.5 (M+1H)⁺. LC purity 99.08% @254 nm.

¹H NMR (400 MHz, DMSO-d₆): δ 10.61 (s, 1H), 8.92 (s, 1H), 7.6 (s, 1H), 7.39-7.30 (m, 6H), 7.18 (t, J=10.4 Hz, 2H), 6.93 (t, J=7.6 Hz, 2H), 5.75 (q, J=6.4 Hz, 1H), 3.72 (d, J=11.2 Hz, 2H), 2.90 (s, 1H), 2.71-2.67 (m, 2H), 2.07 (s, 3H), 1.86-1.75 (m, 4H), 1.63 (s, 2H), 1.52-1.38 (m, 7H), 1.09 (s, 2H).

### Example 24. Preparation of (1R)-1-phenylethyl N-{5-[1-(4-{1-[(3-hydroxy-2-methylpropanesulfonyl)carbamoyl]cyclopropyl}phenyl)piperidin-4-yl]-3-methyl-1,2-oxazol-4-yl}carbamate (Compound 79)

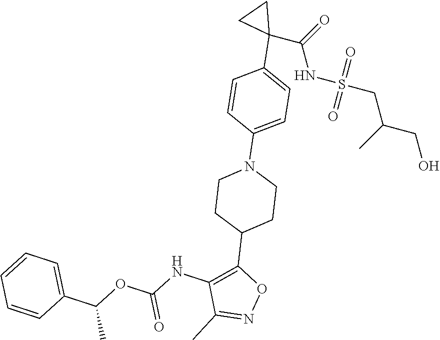 [SMILES: Cc1noc(C2CCN(c3ccc(C4(C(=O)NS(=O)(=O)CC(C)CO)CC4)cc3)CC2)c1NC(=O)O[C@H](C)c1ccccc1]

### Step 1. Preparation of methyl 2-methyl-3-sulfamoylpropanoate

To a solution of methyl 3-(chlorosulfonyl)-2-methylpropanoate (0.5 g, 2.49 mmol) in diethyl ether (10 mL, 96.2 mmol) at 0 °C and purged under ammonia for 30 min and stirred at room temperature 1 hours. The progress of the reaction was monitored by TLC. After completion of the reaction, it was filtered and filterate was concentrated under reduced pressure to afford methyl 2-methyl-3-sulfamoylpropanoate as a gummy liquid. (450 mg, 100%: Yield).

### Step 2. Preparation of methyl 2-methyl-3-({[l-(4-{4-[3-methyl-4-({[(R)-1 phenylethoxy]carbonyl}amino)-1,2-oxazol-5-yl]piperidin-1 yl}phenyl)cyclopropyl]formamido}sulfonyl) propanoate

To a stirred solution of 1-(4-{4-[3-methyl-4-({[(1R)-1-phenylethoxy]carbonyl}amino)-1,2-oxazol-5-yl]piperidin-1-yl}phenyl)cyclopropane-1-carboxylic acid (0.5 g, 1.02 mmol) in THF (5 mL) were added 1-(1H-imidazole-1-carbonyl)-1H-imidazole (248 mg, 1.5 eq., 1.53 mmol) and DMAP (62.4 mg, 0.5 eq., 511 umol) at 0 °C, stirred at 70 °C for 2 hr. Reaction mixture was cooled to rt, Finally added methyl 2-methyl-3-sulfamoylpropanoate (204 mg, 1.1 eq., 1.12 mmol) and DBU (610 μL, 4 eq., 4.09 mmol) at 0 °C stirred at rt for 16 hours. Progress of the reaction was monitored by TLC & LCMS. After completion of reaction, reaction mass was quenched with ice cold water, extracted with ethyl acetate (2×10 mL). The combined organic layer was washed with water (2×10 mL), sodium bicarbonate solution (10 mL) and brine solution (10 mL), dried over anhydrous sodium sulphate, filtered and evaporated under reduced pressure to get crude product.

Crude was given for prep HPLC Mobile phase (A): 0.1% Ammonia in water Mobile phase (B): Acetonitrile to afford methyl 2-methyl-3-({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)propanoate as a white solid (550 mg, 82% Yield); MS (ES) m/z 653.5 (M+H)⁺.

### Step 3. Preparation of (1R)-1-phenylethyl N-{5-[1-(4-{1-[(3-hydroxy-2-methylpropanesulfonyl)carbamoyl]cyclopropyl}phenyl)piperidin-4-yl]-3-methyl-1,2-oxazol-4-yl}carbamate

To a stirred solution of methyl 2-methyl-3-({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)propanoate (150 mg, 230 umol) in tetrahydrofuran (10 mL, 123 mmol) were added LAH (16.1 mg, 2 eq., 460 umol) at 0 °C, allowed to stir at same temperature for 1 hour. Progress of the reaction was monitored by TLC and LCMS. After completion of reaction, reaction mass was quenched with water and ethyl acetate and filtered through celite bed and washed with ethyl acetate, dried with sodium sulphate and concentrated to get crude product. Crude was given for prep HPLC Mobile phase (A): 0.1% Ammonia in water Mobile phase (B): Acetonitrile to afford (1R)-1-phenylethyl N-{5-[1-(4-{1-[(3-hydroxy-2-methylpropanesulfonyl)carbamoyl]cyclopropyl}phenyl)piperidin-4-yl]-3-methyl-1,2-oxazol-4-yl}carbamate as white solid (25 mg, 17% Yield); MS (ES) m/z 625.5 (M+H)⁺. LC purity 99.52% @240 nm.

¹H NMR (400 MHz, DMSO-d₆): δ 8.93 (s, 1H), 7.39-7.07 (m, 5H), 7.31 (d, J=8.4 Hz, 2H), 6.78 (d, J=8.4 Hz, 2H), 5.75 (q, J=6.4 Hz, 1H), 4.41 (d, J=6.4 Hz, 1H), 3.62 (d, J=12 Hz, 2H), 3.28 (d, J=5.2 Hz, 2H), 3.19-3.14 (m, 1H), 2.93-2.85 (m, 2H), 2.79-2.74 (m, 1H), 2.67-2.61 (m, 1H), 2.07 (s, 3H), 1.96-1.90 (m, 1H), 1.88-1.66 (m, 4H), 1.52 (s, 3H), 1.21 (d, J=3.2 Hz, 2H), 0.90 (d, J=6.4 Hz, 3H), 0.68 (d, J=3.2 Hz, 2H).

### Example 25. Preparation of 1-[[1-[4-[4-[1-methyl-5-[[(1R)-1-phenylethoxy]carbonylamino]triazol-4-yl]-1-piperidyl]phenyl]cyclopropanecarbonyl]sulfamoyl]cyclopropanecarboxylic acid (Compound 57)

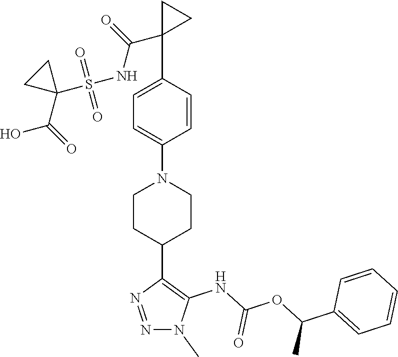 [SMILES: C[C@@H](OC(=O)Nc1c(C2CCN(c3ccc(C4(C(=O)NS(=O)(=O)C5(C(=O)O)CC5)CC4)cc3)CC2)nnn1C)c1ccccc1]

To a solution of ethyl 1-[[1-[4-[4-[1-methyl-5-[[(1R)-1-phenylethoxy]carbonylamino]triazol-4-yl]-1-piperidyl]phenyl]cyclopropanecarbonyl]sulfamoyl]cyclopropanecarboxylate (85 mg, 127.86 mol, 1 eq) in EtOH (2 mL) was added LiOH (2 M, 1 mL, 15.64 eq), then the resulting mixture was stirred at 25 °C for 12 hr. LCMS showed the desired MS was detected and the tert-butyl 4-(5-propoxycarbonyl-1H-triazol-4-yl) piperidine-1-carboxylate consumed. The reaction mixture was acidified to pH=6 by adding AcOH. The reaction mixture was purified by prep-HPLC (Phenomenex luna C₁₈ 150*25 mm*10 um; mobile phase: [water (FA)-ACN]; B %: 29%-59%, 10 min) and lyophilized to give 1-[[1-[4-[4-[1-methyl-5-[[(1R)-1-phenylethoxy]carbonylamino]triazol-4-yl]-1-piperidyl]phenyl]cyclopropanecarbonyl]sulfamoyl]cyclopropanecarboxylicacid (70 mg, 109.94 umol, 85.98% yield, 100% purity) as a white solid.

¹H NMR (400 MHz, methanol-d₄) δ=7.43-7.27 (m, 7H), 7.05 (d, J=8.6 Hz, 2H), 5.83 (q, J=6.7 Hz, 1H), 3.85-3.80 (m, 3H), 3.77 (d, J=12.6 Hz, 2H), 2.85-2.69 (m, 3H), 2.00-1.88 (m, 4H), 1.88-1.84 (m, 2H), 1.67-1.63 (m, 2H), 1.62-1.52 (m, 5H), 1.24-1.16 (m, 2H). MS m/z: 637.4 [M+H]⁺

### Example 26. Preparation of 2-[[1-[4-[4-[1-methyl-5-[[(1R)-1-phenylethoxy]carbonylamino]triazol-4-yl]-1piperidyl]phenyl]cyclopropanecarbonyl]sulfamoyl]acetic acid (Compound 58)

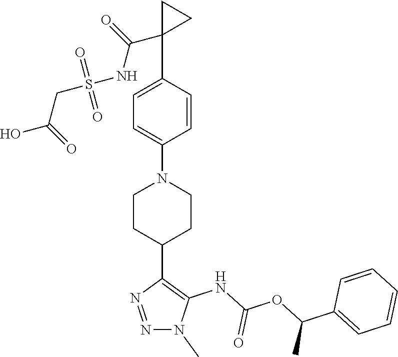 [SMILES: C[C@@H](OC(=O)Nc1c(C2CCN(c3ccc(C4(C(=O)NS(=O)(=O)CC(=O)O)CC4)cc3)CC2)nnn1C)c1ccccc1]

To a solution of methyl 2-[[1-[4-[4-[1-methyl-5-[[(1R)-1-phenylethoxy]carbonylamino]triazol-4-yl]-1-piperidyl]phenyl]cyclopropanecarbonyl]sulfamoyl]acetate (25 mg, 40.02 umol, 1 eq) in MeOH (2 mL) was added LiOH·H₂O (2 M, 0.5 mL, 24.99 eq), then the resulting mixture was stirred at 25 °C for 12 hr. LCMS showed the desired MS was detected and the methyl 2-[[1-[4-[4-[1-methyl-5-[[(1R)-1-phenylethoxy]carbonylamino]triazol-4-yl]-1-piperidyl]phenyl]cyclopropanecarbonyl]sulfamoyl]acetate consumed. The reaction mixture was acidified to pH=6 by adding AcOH. The crude product was purified by prep-HPLC (Unisil 3-100 C₁₈ Ultra 150*50 mm*3 um; mobile phase: [water (FA)-ACN]; B %: 24%-54%, 7 min) to give 2-[[1-[4-[4-[1-methyl-5-[[(1R)-1-phenylethoxy]carbonylamino]triazol-4-yl]-1-piperidyl]phenyl]cyclopropanecarbonyl]sulfamoyl]acetic acid (2.7 mg, 4.29 umol, 10.72% yield, 97% purity) as a white solid.

¹H NMR (400 MHz, methanol-d₄) δ=7.41-7.25 (m, 7H), 7.01 (d, J=8.8 Hz, 2H), 5.85-5.80 (m, 1H), 4.31 (s, 2H), 3.82 (s, 3H), 3.74 (d, J=12.5 Hz, 2H), 2.83-2.71 (m, 3H), 1.98-1.86 (m, 4H), 1.65-1.55 (m, 5H), 1.22-1.16 (m, 2H). MS m/z: 611.2 [M+H]⁺

### Example 27. Preparation of 1-[[1-[4-[4-[5-[[(1R)-1-(2-chlorophenyl)ethoxy]carbonylamino]-1-methyl-triazol-4-yl]-1-piperidyl]phenyl]cyclopropanecarbonyl]sulfamoyl]cyclopropanecarboxylic acid (Compound 39)

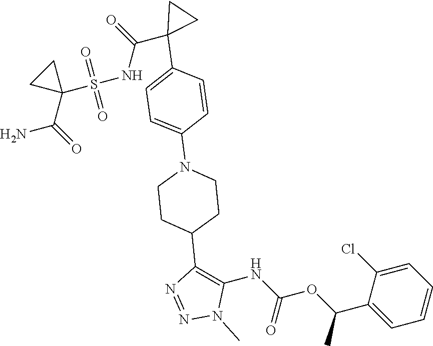 [SMILES: C[C@@H](OC(=O)Nc1c(C2CCN(c3ccc(C4(C(=O)NS(=O)(=O)C5(C(N)=O)CC5)CC4)cc3)CC2)nnn1C)c1ccccc1Cl]

To a solution of ethyl 1-[[1-[4-[4-[5-[[(1R)-1-(2-chlorophenyl)ethoxy]carbonylamino]-1-methyl-triazol-4-yl]-1-piperidyl]phenyl]cyclopropanecarbonyl]sulfamoyl]cyclopropanecarboxylate (50 mg, 71.51 mol, 1 eq) in EtOH (1 mL) was added LiOH (2 M, 0.5 mL, 13.98 eq), then the resulting mixture was stirred at 25 °C for 12 hr. LCMS showed the desired MS was detected and all of ethyl 1-[[1-[4-[4-[5-[[(1R)-1-(2-chlorophenyl)ethoxy]carbonylamino]-1-methyl-triazol-4-yl]-1-piperidyl]phenyl]cyclopropanecarbonyl]sulfamoyl]cyclopropanecarboxylate consumed. The reaction mixture was acidified to pH=6 by adding AcOH, then the resulting mixture was purified by Prep-HPLC (Phenomenex luna C18 150*25 mm*10 um; mobile phase: [water(FA)-ACN]; gradient: 366%-66% B over 10 min) to give 1-[[1-[4-[4-[5-[[(1R)-1-(2-chlorophenyl)ethoxy]carbonylamino]-1-methyl-triazol-4-yl]-1-piperidyl]phenyl]cyclopropanecarbonyl]sulfamoyl]cyclopropanecarboxylic acid (15.7 mg, 22.46 mol, 31.40% yield, 96% purity) as a white solid.

¹H NMR (400 MHz, methanol-d₄) δ=7.59 (d, J=3.9 Hz, 1H), 7.41 (d, J=8.8 Hz, 1H), 7.36-7.28 (m, 4H), 7.05 (d, J=8.7 Hz, 2H), 6.21-6.15 (m, 1H), 3.85 (s, 3H), 3.80 (d, J=12.2 Hz, 2H), 2.88-2.76 (m, 3H), 1.98-1.90 (m, 4H), 1.88-1.84 (m, 2H), 1.66-1.63 (m, 2H), 1.62-1.57 (m, 3H), 1.57-1.54 (m, 2H), 1.22-1.18 (m, 2H). MS m/z: 671.2 [M+H]⁺

### Example 28. Preparation of 3-({[1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl)cyclopropyl]formamido}sulfonyl)propanoic acid (Compound 31)

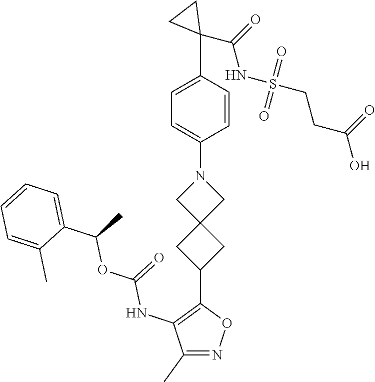 [SMILES: Cc1ccccc1[C@@H](C)OC(=O)Nc1c(C)noc1C1CC2(C1)CN(c1ccc(C3(C(=O)NS(=O)(=O)CCC(=O)O)CC3)cc1)C2]

### Step 1. Preparation of methyl 3-({[1-(4-{6-13-methyl-4-({[(1R)-1-(2-methylphenyl)ethoxy]carbonyl}amino)-1,2-oxazol-5-yl]-2-azaspiro[3.3]heptan-2-yl}phenyl)cyclopropyl]formamido}sulfonyl)propanoate

To a stirred solution 1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl) cyclopropane-1-carboxylic acid (130 mg, 252 umol) in DCM (2 mL) were added Triethylamine (105 μL, 3 eq., 756 umol), DMAP (15.4 mg, 0.5 eq., 126 umol), EDC·HCl (82.1 mg, 3 eq., 756 umol) and HOBT (68.1 mg, 2 eq., 504 mol), stirred at room temperature for 15 minutes. Finally added methyl 3-sulfamoylpropanoate (126 mg, 3 eq., 756 umol) and stirred at rt for 12 hours. Progress of the reaction was monitored by TLC & LCMS. After completion of reaction, reaction mass was quenched with ice cold water, extracted with ethyl acetate (2×10 mL). The combined organic layer was washed with water (2×10 mL), sodium bicarbonate solution (10 mL) and brine solution (5 mL), dried over anhydrous sodium sulphate, filtered and evaporated under reduced pressure to get crude product methyl 3-({[1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl)cyclopropyl]formamido}sulfonyl)propanoate (Crude) as white solid; MS (ES) m z 665.3 (M+1H).

¹H NMR (400 MHz, DMSO-d₆): δ 8.88 (s, 1H), 7.42 (d, J=6.8 Hz, 1H), 7.24-7.18 (m, 3H), 7.03 (d, J=8.4 Hz, 2H), 6.28 (d, J=8.4 Hz, 2H), 5.90 (q, J=6.8 Hz, 1H), 3.82 (s, 2H), 3.66 (s, 2H), 3.58 (s, 3H), 3.45-3.40 (m, 1H), 3.24-3.17 (m, 1H), 3.15-3.06 (m, 1H), 3.09-3.04 (m, 2H), 3.03-2.95 (m, 1H), 2.46 (s, 1H), 2.38 (s, 3H), 2.20-2.17 (m, 1H), 2.10 (s, 3H), 2.03-1.96 (m, 1H), 1.74-1.61 (m, 1H), 1.36 (s, 3H), 1.24 (d, J=3.2 Hz, 2H). 0.71 (d, J=2.4 Hz, 2H).

### Step 2. Preparation of 3-({[1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl)cyclopropyl]formamido}sulfonyl)propanoic acid

To a stirred solution of methyl 3-({[1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl)cyclopropyl]formamido}sulfonyl)propanoate (60 mg, 90.3 umol) in oxolane (2 mL), methanol (2 mL, 156 mmol) solution was added lithium(1+) hydrate hydroxide (15.1 mg, 4 eq., 361 mol) in water (2 mL) and the mixture was stirred at RT for 18 hours. Reaction was progressed by LCMS and TLC, rm was concentrated, diluted with water (10 mL), washed with diethyl ether (2×10 mL), PH was adjusted with 10% Aq. citric acid up to 3 to 4, white solid was precipitated, filtered on buchnel funnel, solid was washed with water (2×10 mL), pentane (2×10 mL) and dried under vacuum to afford 3-({[1-(4-{6-[3-methyl-4-({[(1R)-1-(2-methylphenyl)ethoxy]carbonyl}amino)-1,2-oxazol-5-yl]-2-azaspiro[3.3]heptan-2-yl}phenyl)cyclopropyl]formamido}sulfonyl)propanoic acid as off white solid (35 mg, 60% yield); MS (ES) m/z 651.3 (M+1H)⁺. LC purity 98.42% @240 nm.

¹H NMR (400 MHz, DMSO-d₆): δ 12.55 (s, 1H), 10.72 (s, 1H), 8.87 (s, 1H), 7.42 (d, J=6.4 Hz, 1H), 7.24-7.09 (m, 5H), 6.36 (q, J=8.4 Hz, 2H), 5.91 (q, J=6.4 Hz, 1H), 3.85 (d, J=7.6 Hz, 2H), 3.70 (d, J=8.4 Hz, 2H), 3.54 (t, J=7.2 Hz, 2H), 3.48-3.41 (m, 1H), 2.61 (t, J=6.8 Hz, 2H), 2.47-2.39 (m, 4H), 2.33 (s, 3H), 2.07 (s, 3H), 1.58 (s, 3H), 1.34 (s, 2H), 1.02 (s, 2H).

### Example 29. Preparation of 3-[[1-[3-methoxy-4-[4-[3-methyl-4-[[(1R)-1-phenylethoxy]carbonylamino]isoxazol-5-yl]-1-piperidyl]phenyl]cyclopropanecarbonyl]sulfamoyl]propanoic acid (Compound 1)

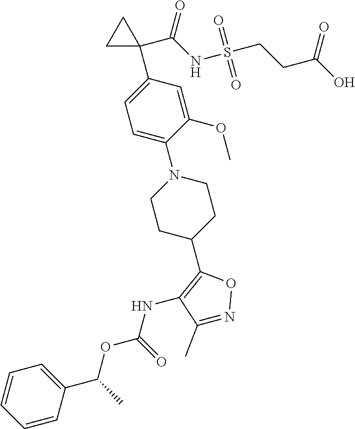 [SMILES: COc1cc(C2(C(=O)NS(=O)(=O)CCC(=O)O)CC2)ccc1N1CCC(c2onc(C)c2NC(=O)O[C@H](C)c2ccccc2)CC1]

### Step 1. Preparation of dimethyl 2-(4-bromo-3-methoxy-phenyl)propanedioate

To a solution of 1-bromo-4-iodo-2-methoxy-benzene(2A) (10 g, 31.96 mmol, 1 eq) and dimethyl propanedioate(2B) (23.00 g, 174.09 mmol, 20 mL, 5.45 eq) in dioxane (200 mL) was added CuI (1.2 g, 6.30 mmol, 1.97e−1 eq), pyridine-2-carboxylic acid (1.6 g, 13.00 mmol, 4.07e−1 eq) and Cs₂CO₃ (30 g, 92.08 mmol, 2.88 eq), the reaction mixture was stirred at 110 °C for 22 hrs. TLC (PE/EtOAc=2/1, Rf=0.24) showed one major spot was observed. The reaction was extracted with EtOAc (500 mL*3). The combined organic phase was washed with saturated brine (500 mL*2), dried over anhydrous Na₂SO₄, filtered and concentrated in vacuo. The residue was purified by flash silica gel chromatography (ISCO®; 80 g SepaFlash® Silica Flash Column, Eluent of 0-10% Ethyl acetate/Petroleum ether gradient @60 mL/min) to afford dimethyl 2-(4-bromo-3-methoxy-phenyl) propanedioate(2C) (10 g, 31.53 mmol, 98.67% yield) as yellow oil.

¹H NMR (400 MHz, chloroform-d) δ=7.40 (d, J=8.0 Hz, 1H), 6.76 (d, J=1.8 Hz, 1H), 6.68 (d, J=1.9, 8.1 Hz, 1H), 3.83 (s, 3H), 3.63 (s, 3H), 3.52 (s, 2H).

### Step 2. Preparation of methyl 2-(4-bromo-3-methoxy-phenyl)acetate

To a solution of dimethyl 2-(4-bromo-3-methoxy-phenyl) propanedioate(2C) (7 g, 22.07 mmol, 1 eq) in DMSO (100 mL) was added H2O (3.50 g, 194.28 mmol, 3.5 mL, 8.80 eq) and LiCl (3 g, 70.76 mmol, 1.45 mL, 3.21 eq), the reaction mixture was stirred at 110 °C for 12 hr. TLC (PE/EtOAc=5/1, Rf=0.24) showed one major spot was observed. The reaction was extracted with EtOAc (300 mL*3). The combined organic phase was washed with sat. NH4Cl (100 mL*3) and saturated brine (300 mL*2), dried over anhydrous Na₂SO4, filtered and concentrated in vacuo to give a residue. The residue was purified by flash silica gel chromatography (ISCO®; 40 g SepaFlash® Silica Flash Column, Eluent of 0~10% Ethyl acetate/Petroleum ether gradient @40 mL/min) to afford methyl 2-(4-bromo-3-methoxy-phenyl) acetate(2D) (5.4 g, 20.84 mmol, 94.42% yield) as yellow oil.

¹H NMR (400 MHz, chloroform-d) δ=7.40 (d, J=8.0 Hz, 1H), 6.76 (d, J=1.8 Hz, 1H), 6.68 (d, J=1.8, 8.0 Hz, 1H), 3.83 (s, 3H), 3.63 (s, 3H), 3.52 (s, 2H).

### Step 3. Preparation of methyl1-(4-bromo-3-methoxy-phenyl)cyclopropanecarboxylate

To a solution of methyl 2-(4-bromo-3-methoxy-phenyl) acetate(2D) (5.4 g, 20.84 mmol, 1 eq) in THF (100 mL) was added NaHMDS (1 M, 60 mL, 2.88 eq) at −40 °C and stirred for 1 hr, then the resulting mixture was added 1,3,2-dioxathiolane 2,2-dioxide(2E) (5.4 g, 43.51 mmol, 2.09 eq) at −40 °C and stirred for 1 hr. TLC (PE/EtOAc=10/1, Rf=0.58) showed one major spot was observed. sat. NH4Cl (10 mL) was added to the solution and keep the temperature from 0 °C to 5 °C, then the reaction was extracted with EtOAc (20 mL*3). The combined organic phase was washed with saturated brine (20 mL*2), dried over anhydrous Na2SO4, filtered and concentrated in vacuo to give a residual. The residual was purified by flash silica gel chromatography (ISCO®; 40 g SepaFlash® Silica Flash Column, Eluent of 0~10% Ethyl acetate/Petroleum ether gradient @40 mL/min) to afford methyl 1-(4-bromo-3-methoxy-phenyl) cyclopropanecarboxylate (1.7 g, 5.96 mmol, 28.61% yield) as colorless oil.

¹H NMR (400 MHz, chloroform-d) δ=7.31 (d, J=8.4 Hz, 1H), 6.75 (d, J=1.8 Hz, 1H), 6.68 (d, J=1.8, 8.4 Hz, 1H), 3.75 (s, 3H), 3.49 (s, 3H), 1.48-1.46 (m, 2H), 1.06-1.03 (m, 2H).

### Step 4. Preparation of methyl 1-[3-methoxy-4-[4-[3-methyl-4-[[(1R)-1-phenylethoxy]carbonylamino]isoxazol-5-yl]-1-piperidyl]phenyl]cyclopropane-carboxylate

To a solution of [(1R)-1-phenylethyl]N-[3-methyl-5-(4-piperidyl) isoxazol-4-yl]carbamate (150 mg, 455.38 umol, 1 eq) in dioxane (2 mL) was added methyl 1-(4-bromo-3-methoxy-phenyl) cyclopropanecarboxylate (240.00 mg, 841.71 mol, 1.85 eq), Cs2CO3 (450.00 mg, 1.38 mmol, 3.03 eq) and CPHOS Pd G3 (40 mg, 49.61 mol, 1.09e−1 eq), then the resulting mixture was purged with N2 for three times and stirred at 90 °C for 12 hr. LCMS showed the desired MS was detected and the [(1R)-1-phenylethyl]N-[3-methyl-5-(4-piperidyl) isoxazol-4-yl]carbamate consumed. TLC (PE/EA=3:1, Rf=0.3) showed a new spot was detected and the [(1R)-1-phenylethyl]N-[3-methyl-5-(4-piperidyl) isoxazol-4-yl]carbamate consumed. The reaction mixture was partitioned between EtOAc (80 mL) and water (60 mL), washed with brine (60 mL), dried over Na2SO4, filtered and evaporated under reduced pressure to give the crude product. The crude product was purified by prep-TLC (SiO2, PE:EA=3:1) to give methyl 1-[3-methoxy-4-[4-[3-methyl-4-[[(1R)-1-phenylethoxy]carbonylamino]isoxazol-5-yl]-1-piperidyl]phenyl]cyclopropane-carboxylate (130 mg, 168.10 mol, 36.91% yield, 69% purity) as a white solid. MS m/z: 534.3 [M+H]+

### Step 5. Preparation of 1-[3-methoxy-4-[4-[3-methyl-4-[[(R)-1-phenylethoxy]carbonylamino]isoxazol-5-yl]-1-piperidyl]phenyl]cyclopropanecarboxylic acid

To a solution of methyl 1-[3-methoxy-4-[4-[3-methyl-4-[[(1R)-1-phenylethoxy]carbonylamino]isoxazol-5-yl]-1-piperidyl]phenyl]cyclopropanecarboxylate (120 mg, 224.88 umol, 1 eq) in MeOH (2 mL) was added LiOH (2 M, 1 mL, 8.89 eq), then the resulting mixture was stirred at 25 °C for 12 hr. LCMS showed the desired MS was detected and the methyl 1-[3-methoxy-4-[4-[3-methyl-4-[[(1R)-1-phenylethoxy]carbonylamino]isoxazol-5-yl]-1-piperidyl]phenyl]cyclopropanecarboxylate consumed. The reaction mixture was acidified to pH=6 by adding AcOH, partitioned between EtOAc (60 mL) and water (40 mL), washed with brine (40 mL), dried over Na2SO4, filtered and evaporated under reduced pressure to give crude 1-[3-methoxy-4-[4-[3-methyl-4-[[(1R)-1-phenylethoxy]carbonylamino]isoxazol-5-yl]-1-piperidyl]phenyl]cyclopropanecarboxylic acid (100 mg, 192.46 mol, 85.58% yield) as the yellow oil which was used into the step without further purification. MS m/z: 520.3 [M+H]+

### Step 6. Preparation of methyl 3-[[1-[3-methoxy-4-[4-[3-methyl-4-[[(1R)-1-phenylethoxy]carbonylamino]isoxazol-5-yl]-1-piperidyl]phenyl]cyclopropanecarbonyl]sulfamoyl]propanoate

To a solution of 1-[3-methoxy-4-[4-[3-methyl-4-[[(1R)-1-phenylethoxy]carbonylamino]isoxazol-5-yl]-1-piperidyl]phenyl]cyclopropanecarboxylic acid (70 mg, 134.72 mol, 1 eq) in DCM (3 mL) was added methyl 3-sulfamoylpropanoate (50 mg, 299.07 umol, 2.22 eq), EDCI (120 mg, 625.97 umol, 4.65 eq) and DMAP (70 mg, 572.98 umol, 4.25 eq), then the resulting mixture was stirred at 25 °C for 12 hr. LCMS showed the desired MS was detected and the 1-[3-methoxy-4-[4-[3-methyl-4-[[(1R)-1-phenylethoxy]carbonylamino]isoxazol-5-yl]-1-piperidyl]phenyl]cyclopropanecarboxylic acid consumed. TLC (PE/EA=1:1, Rf=0.3) showed a new spot was detected and the 1-[3-methoxy-4-[4-[3-methyl-4-[[(1R)-1-phenylethoxy]carbonylamino]isoxazol-5-yl]-1-piperidyl]phenyl]cyclopropanecarboxylic acid consumed. The reaction mixture was partitioned between EtOAc (70 mL) and HCl (0.1N, 40 mL), washed with brine (50 mL), dried over Na2SO4, filtered and evaporated under reduced pressure to give the crude product. The crude product was purified by prep-TLC (SiO2, PE:EA=1:1) to give methyl 3-[[1-[3-methoxy-4-[4-[3-methyl-4-[[(1R)-1-phenylethoxy]carbonylamino]isoxazol-5-yl]-1-piperidyl]phenyl]cyclopropanecarbonyl]sulfamoyl]propanoate (45 mg, 63.25 mol, 46.95% yield, 94% purity) as a white solid. MS m/z: 669.4 [M+H]+.

### Step 7. Preparation of 3-[[1-[3-methoxy-4-[4-[3-methyl-4-[[(1R)-1-phenylethoxy]carbonylamino]isoxazol-5-yl]--piperidyl]phenyl]cyclopropanecarbonyl]sulfamoyl]propanoic acid

To a solution of methyl 3-[[1-[3-methoxy-4-[4-[3-methyl-4-[[(1R)-1-phenylethoxy]carbonylamino]isoxazol-5-yl]-1-piperidyl]phenyl]cyclopropanecarbonyl]sulfamoyl]propanoate (40 mg, 59.81 mol, 1 eq) in MeOH (2 mL) was added LiOH (2 M, 1 mL, 33.44 eq), then the resulting mixture was stirred at 25 °C for 12 hr. LCMS showed the desired MS was detected and the methyl 3-[[1-[3-methoxy-4-[4-[3-methyl-4-[[(1R)-1-phenylethoxy]carbonylamino]isoxazol-5-yl]-1-piperidyl]phenyl]cyclopropanecarbonyl]sulfamoyl]propanoate consumed. The reaction mixture was acidified to pH=6 by adding AcOH. The reaction mixture was purified by prep-HPLC (Phenomenex luna C18 150*25 mm*10 um; mobile phase: [water (FA)-ACN]; B %: 26%-56%, 10 min) to give the 3-[[1-[3-methoxy-4-[4-[3-methyl-4-[[(1R)-1-phenylethoxy]carbonylamino]isoxazol-5-yl]-1-piperidyl]phenyl]cyclopropanecarbonyl]sulfamoyl]propanoic acid (11.29 mg, 17.24 mol, 28.83% yield, 100% purity) as a white solid.

¹H NMR (400 MHz, methanol-d₄) δ=7.42-7.26 (m, 5H), 7.03-6.96 (m, 3H), 5.83-5.75 (m, 1H), 3.90 (s, 3H), 3.66 (t, J=7.1 Hz, 2H), 3.49-3.42 (m, 2H), 2.91-2.82 (m, 1H), 2.73 (t, J=7.2 Hz, 2H), 2.69-2.57 (m, 2H), 2.11 (s, 3H), 2.05-1.86 (m, 4H), 1.57 (m, 5H), 1.24-1.18 (m, 2H). MS m/z: 655.2 [M+H]+.

### Example 30. Preparation of 2-methyl-2-[[1-[4-[4-[3-methyl-4-[[(1R)-1-phenylethoxy]carbonylamino]isoxazol-5-yl]-1-piperidyl]phenyl]cyclopropanecarbonyl]sulfamoyl]propanoic acid (Compound 67)

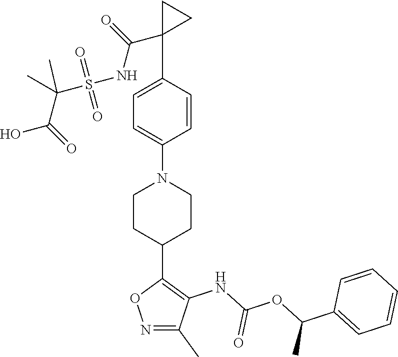 [SMILES: Cc1noc(C2CCN(c3ccc(C4(C(=O)NS(=O)(=O)C(C)(C)C(=O)O)CC4)cc3)CC2)c1NC(=O)O[C@H](C)c1ccccc1]

To a solution of methyl 2-methyl-2-[[1-[4-[4-[3-methyl-4-[[(1R)-1-phenylethoxy]carbonylamino]isoxazol-5-yl]-1-piperidyl]phenyl]cyclopropanecarbonyl]sulfamoyl]propanoate (50 mg, 76.60 umol, 1 eq) in MeOH (2 mL) was added LiOH·H₂O (2 M, 0.5 mL, 13.06 eq), then the resulting mixture was stirred at 25 °C for 12 hr. LCMS showed the methyl 2-methyl-2-[[1-[4-[4-[3-methyl-4-[[(1R)-1-phenylethoxy]carbonylamino]isoxazol-5-yl]-1-piperidyl]phenyl]cyclopropanecarbonyl]sulfamoyl]propanoate (50 mg, 76.60 mol, 1 eq) consumed and desired MS was detected. The reaction mixture was acidified to pH=6 by adding AcOH. The reaction mixture was purified by prep-HPLC (Unisil 3-100 C₁₈ Ultra 150*50 mm*3 um; mobile phase: [water (FA)-ACN]; B %: 46%-76%, 7 min) to give the 2-methyl-2-[[1-[4-[4-[3-methyl-4-[[(1R)-1-phenylethoxy]carbonylamino]isoxazol-5-yl]-1-piperidyl]phenyl]cyclopropanecarbonyl]sulfamoyl]propanoic acid (15.1 mg, 23.64 umol, 30.86% yield, 100% purity) as a white solid.

¹H NMR (400 MHz, methanol-d₄) δ=7.43-7.28 (m, 7H), 7.03 (d, J=8.4 Hz, 2H), 5.84-5.74 (m, 1H), 3.75 (d, J=12.6 Hz, 2H), 2.99-2.90 (m, 1H), 2.85-2.72 (m, 2H), 2.11 (s, 3H), 1.95 (m, 4H), 1.58 (s, 9H), 1.56-1.53 (m, 2H), 1.24-1.17 (m, 2H). MS m/z: 639.2 [M+H]⁺

### Example 31. Preparation of (R)-2,2-dimethyl-3-(N-(1-(4-(4-(3-methyl-4-(((1-phenylethoxy)carbonyl)amino)isoxazol-5-yl)piperidin-1-yl)phenyl)cyclopropane-1-carbonyl)sulfamoyl)propanoic acid (Compound 78)

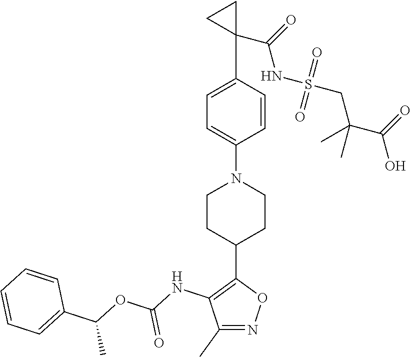 [SMILES: Cc1noc(C2CCN(c3ccc(C4(C(=O)NS(=O)(=O)CC(C)(C)C(=O)O)CC4)cc3)CC2)c1NC(=O)O[C@H](C)c1ccccc1]

To a stirred solution of methyl 2,2-dimethyl-3-({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)propanoate (25 mg, 37.5 umol) in THF (3.0 mL), MeOH (3.0 mL) solution was added lithium(1+) hydrate hydroxide (6.29 mg, 150 umol) in water (3.0 mL) and the mixture was stirred at rt for 18 hours. The progress of the reaction was monitored by LCMS and TLC. After completion of the reaction, the reaction mixture was concentrated, diluted with water (10 mL), washed with diethyl ether (2×5 mL), pH was adjusted with 10% Aq. citric acid up to 3 to 4, white solid was precipitated, filtered on buchnel funnel, solid was washed with water (2×5 mL), pentane (2×5 mL) and dried under vacuum to afford 2,2-dimethyl-3-({[1-(4-{4-[3-methyl-4-({[(1R)-1-phenylethoxy]carbonyl}amino)-1,2-oxazol-5-yl]piperidin-1-yl}phenyl)cyclopropyl]formamido}sulfonyl)propanoic acid as an off white solid (5 mg, 21% yield); MS (ES) m/z 653.5 (M+1H)⁺. LC purity 99.43% @240 nm. ¹H NMR (400 MHz, DMSO-d₆): δ 12.54 (s, 1H), 10.88 (s, 1H), 8.92 (s, 1H), 7.39-7.30 (m, 5H), 7.16 (d, J=8.8 Hz, 2H), 6.91 (d, J=8.8 Hz, 2H), 5.74 (q, J=6.4 Hz, 1H), 3.72 (d, J=12.0 Hz, 2H), 3.64 (s, 2H), 2.91-2.89 (m, 1H), 2.74-2.66 (m, 2H), 2.04 (s, 3H), 1.86-1.75 (m, 4H), 1.52 (d, J=5.6 Hz, 3H), 1.39 (s, 2H), 1.23 (m, 6H), 1.06 (s, 2H).

### Additional Exemplary Compounds of Formula (I)

Compounds 3-7, 9, 11-18, 20, 22, 25, 28-30, 32, 34-36, 40-45, 47-53, 55, 56, 59, 61, 62, 64-66, 68, 70-72, 75-77, 80, 81, 83-86, 90, 91, and 93-97 were synthesized analogously to compounds 1, 2, 8, 10, 19, 21, 23, 24, 26, 27, 31, 33, 37-39, 46, 54, 57, 58, 60, 63, 67, 69, 73, 74, 78, 79, 82, 87-89, and 92 using modifications within the purview of one having skill in the art.

### Biological Examples

### Ca²⁺ Flux Assay

### Procedure

B103 cells stably expressing hLPAR1 were seeded at a density of 1.0×10⁵ cells per well in clear flat-bottom black 96-well plates and incubated overnight in complete media. The following day, the cells were pre-incubated in serum-free media for 2 hours prior to ensure cellular equilibration. For the loading a calcium-sensitive fluorescent dye, the cells were treated with a concentration of 5 μM Fura-2 AM dye (Sigma, F1225) in Hank's buffered salt solution (HBSS) supplemented with 20 mM HEPES, 1 M probenecid, and 0.3% fatty-acid-free bovine serum albumin for 40 minutes at room temperature (RT). Test compounds, prepared in DMSO, were then added to each well and incubated for 20 minutes at RT. To induce calcium release, a concentration of 5 M LPA in HBSS supplemented with 20 mM HEPES, and 0.3% fatty-acid-free bovine serum albumin was introduced. Intracellular calcium mobilization was subsequently quantified by monitoring the fluorescence intensity using the Spark® multimode microplate reader (TECAN).

### Chemotaxis Assay

### Procedure

For monitoring the migration of cells, Neuroprobe ChemoTx® System plates (10 mm pore size, 5.7 mm diameter sites; Gaithersburg, MD, USA) were utilized. To induce cell migrations, a concentration of 5 M LPA or vehicle in DMEM were loaded to the bottom chamber. A total of 15,000 A2058 cells, which were starved for 24 hr following treated with test compounds or vehicle, were then applied to the upper membrane coated with 0.001% fibronectin. The plate was incubated for 4 to 8 hours to allow cell migration. Following incubation, the plate was dissembled, and the migrated cells were visualized by crystal violet staining. The stained area with crystal violet was quantified by measuring the absorbance at 590 nm and the relative percentage of the control was calculated.

### Bile Salt Export Pump (BSEP) Assay

### A. Test Article

1. A stock solution was prepared with the test article (Sponsor compound) in DMSO.
2. All final assay solutions of probe substrate, reference inhibitor or test article contain 0.5% (v/v) DMSO (vehicle control).
3. Test article concentrations in transport study were specified in the Summary Table of Experimental Conditions of this protocol.

TABLE 1
Summary of Experimental Conditions:

| Test System | Sf9 insect cell vesicles expressing BSEP |
|---|---|
| Probe Substrate (positive | BSEP: 1 μM [3H]-taurocholic acid |
| control for transport) | |
| Reference Inhibitor (Positive | BSEP: 300 μM rifampicin |
| Control for Inhibition) | |
| Test Article Concentration(s) | 10 μM |
| Pre-incubation Time (min) | 10 |
| Incubation Time (min) | 15 |

### B. Materials

1. Bovine Serum Albumin (BSA)
2. Dimethyl sulfoxide (DMSO)
3. BSEP Assay Uptake Buffer (10 mM HEPES-Tris pH 7.4, 0.1 M KNO₃, 12.5 mM Mg(NO₃)₂, 50 mM sucrose)
4. BSEP Wash Buffer (10 mM Tris-HCl pH 7.4, 0.1 M KNO₃, 50 mM sucrose)
5. BSEP Blocking Buffer (BSEP Wash Buffer+0.5 mg/mL BSA)
6. 25 mM Mg-ATP solution
7. 25 mM AMP solution
8. Corning Costar 96-well flat-bottom, untreated, white Assay Plate (Corning Cat. No. 3912)
9. Millipore MultiscreenHTs Filtration Plate, glass fiber FB—1.0/0.65 μm (Cat. No. MSFBN6B10 or glass fiber FC—1.2/0.65 μm (Cat. No. MSFCN6B10)
10. Millipore MultiscreenHTS Vacuum Manifold (Cat. No. MSVMHTS00)
11. Probe substrates listed in Table 1
12. Reference inhibitors listed in Table 1
13. Test articles

### C. Transport Study for BSEP

1. Test system:
   a. 96-well flat bottom plate containing a suspension of vesicles and a corresponding 96-well glass fiber filtration plate.
   b. Transport experiments were conducted in the Assay Uptake Buffer.
   c. Transport experiment was initiated with the addition of Mg-ATP or AMP to appropriate wells.
   d. Each condition was run in triplicate wells.
2. Prepared the 96-well flat-bottom assay plate.
   a. Incubated the assay plate with the Blocking Buffer for 60 min at 37 °C with orbital shaking at approximately 90 rpm. (NOTE: This is done to prevent non-specific binding of vesicles to polystyrene assay plate).
   b. After 60 min, completely removed the Blocking Buffer from the assay plate.
   c. The Assay Uptake Buffer solutions were added to the assay plate wells as follows:
      i. For probe substrate transport assays, the Assay Uptake Buffer contained vesicles and the probe substrate at the specified concentration.
      ii. For the reference inhibition assay, the Assay Uptake Buffer contained vesicles, the reference inhibitor and the probe substrate at the specified concentration in Table 1.
      iii. For the test article assays, Assay Uptake Buffer contained vesicles, the test article and the probe substrate at the specified concentration in Table 1.
3. Incubate at 37 °C with orbital shaking at approximately 90 rpm for the time period specified in Table 1 of this protocol.
4. Using the 25 mM Mg-ATP or AMP stocks, Mg-ATP or AMP was added to test wells of flat bottom assay plate to a final concentration of 5 mM.
5. Incubated at 37 °C with orbital shaking at approximately 90 rpm for the incubation time specified in Table 1 of this protocol.
6. Prepared the 96-well glass fiber filtration plate:
   a. Incubated the filtration plate with the Blocking Buffer for 2 min at room temperature with under no vacuum.
   b. Turned on vacuum and allowed Blocking Buffer to filter through plate.
   c. With vacuum on, filter plate was washed with ice cold Wash Buffer.
7. At the end of the specified incubation time indicated in Table is of this protocol, vesicle transport was quenched by adding ice cold Wash Buffer to samples in 96-well assay plate.
8. Mixed assay samples (now diluted in Wash Buffer) with pipette and transfered 175 μL into the 96-well glass fiber filtration plate to separate vesicles from buffer by applying vacuum.
9. Washed filter plate wells with ice cold Wash Buffer.
10. Allowed filter plate to dry under vacuum.
11. Transfered filters to scintillation sample plate and added 700 μL of scintillation fluid to the above samples.
12. Counted the above samples on a 1450 Microbeta (Perkin-Elmer).

### D. Analytical, Data Analysis and Reporting

1. The amount of substrate was quantified with radiometric detection on a 1450 Microbeta (Perkin-Elmer).
2. For transport study samples run in triplicate, the mean and standard deviation of the replicates were reported.
3. For BSEP, the ATP-dependent substrate transport rate (V) in Sf9 vesicles containing the transporter were calculated by subtracting the mean substrate accumulation in vesicles treated with AMP from the substrate accumulation in vesicles treated with ATP:

ATP-dependent transport rate(pmol/min/mg)=((Vesicular accumulation)_{ATP}−(Mean vesicular accumulation)_{AMP})/Incubation time
4. Percent inhibition in the presence of the test article or the reference inhibitors was calculated as follows:

Percent inhibition=100−(100*(ATP-dependent transport rate)_{with inhibitor}/(ATP-dependent transport rate)_{vehicle control})
5. For vesicle assays, statistical analysis using unpaired t test was performed between transporter-mediated uptake rate or ATP-dependent transport rate of probe substrate with vehicle control and the inhibitor. A p value <0.05 is considered statistically significant.
6. All reported data was based on nominal concentration used unless otherwise stated.
7. Data and calculated values were displayed at 3 significant figures with rounding occurring only for final display.
8. BSEP % inhibition is at 10 μM of test compound
9. BSEP inhibition was determined at BioIVT.

The following table, Table B, lists the results of exemplary compounds tested in the Ca²⁺ Flux and Chemotsaxis assays as described the foregoing, with the IC₅₀ values listed in ranges according to: Ca²⁺ Assay IC₅₀: D≥3000 nM>C≥200 nM>B≥10 nM>A; Chemotaxis IC₅₀: D≥5000 nM>C≥500 nM>B≥100 nM>A. Liver microsomal stability is percent of parent compound remaining after 30 minutes incubation with mouse (M), rat (R), or human (H) liver microsomes with percent ranges of D≤25%<C≤50%<B≤75%<A. BSEP % inhibition is at 10 μM of test compound, with percent inhibition ranges of A≤25%<B≤50%<C≤75%<D≤100%.

TABLE B

| | | | Liver Microsomal | | | |
|---|---|---|---|---|---|---|
| | | | stability (%) | | | |
| | Calcium | | *Buspirone | | | |
| | Assay | Chemotaxis | % of remaining | | | BSEP |
| | IC₅₀ | IC₅₀ | after 30 min | | | inhibition |
| Compound # | (nM) | (nM) | Mouse | Rat | Human | at 10 μM |
| R-1 | B | A | A | A | A | D |
| R-2 | B | C | | | | D |
| 1 | A | A | | | | |
| 2 | A | A | | | | |
| 3 | A | D | | | | |
| 4 | A | C | | | | |
| 5 | B | C | | | | |
| 6 | B | C | | A | A | |
| 7 | A | A | | | | D |
| 8 | A | B | | B | B | C |
| 9 | A | B | | | | C |
| 10 | B | A | | A | A | B |
| 11 | B | B | | | | |
| 12 | B | A | | B | A | B |
| 13 | D | D | | B | D | |
| 14 | A | A | | | | |
| 15 | C | C | | | | |
| 16 | B | C | | | | |
| 17 | A | C | | | | A |
| 18 | C | C | | | | |
| 19 | A | A | | | | C |
| 20 | B | B | | | | D |
| 21 | B | A | | A | A | |
| 22 | B | B | | B | B | |
| 23 | B | C | | A | A | B |
| 24 | A | B | | | | |
| 25 | B | D | | A | A | |
| 26 | A | C | | | | |
| 27 | A | A | | | | |
| 28 | B | C | | | | |
| 29 | C | C | | | | |
| 30 | B | C | | | | |
| 31 | B | A | | | | |
| 32 | C | D | | | | |
| 33 | A | A | | | | D |
| 34 | B | A | | | | |
| 35 | A | A | | B | C | |
| 36 | A | A | | | | |
| 37 | A | A | | | | C |
| 38 | A | A | | A | C | C |
| 39 | A | A | | | | |
| 40 | B | B | | | | |
| 41 | A | B | | | | |
| 42 | B | A | | | | |
| 43 | B | B | | | | |
| 44 | B | B | | | | |
| 45 | B | B | | | | |
| 46 | A | C | | | | |
| 47 | C | C | | | | |
| 48 | B | A | | | | |
| 49 | B | C | | | | |
| 50 | A | A | | | | |
| 51 | A | C | | | | |
| 52 | A | A | | | | |
| 53 | A | C | | | | |
| 54 | B | A | | | | B |
| 55 | B | B | | | | B |
| 56 | A | B | | | | |
| 57 | A | A | | | | |
| 58 | B | B | | | | A |
| 59 | A | B | | | | A |
| 60 | B | B | | | | A |
| 61 | A | A | | | | C |
| 62 | B | B | | A | A | B |
| 63 | A | A | | | | A |
| 64 | A | A | | C | B | D |
| 65 | B | B | | | | C |
| 66 | A | A | | | | |
| 67 | A | A | | A | A | B |
| 68 | A | A | | | | |
| 69 | A | A | | | | |
| 70 | A | A | | A | A | |
| 71 | A | A | | | | B |
| 72 | A | A | | | | A |
| 73 | A | A | | | | |
| 74 | A | A | | | | C |
| 75 | A | A | | | | |
| 76 | A | B | | | | |
| 77 | A | A | | | | |
| 78 | A | A | | | | B |
| 79 | A | A | | | | B |
| 80 | A | A | | B | A | B |
| 81 | A | A | | | | |
| 82 | A | A | | | | |
| 83 | A | A | | A | A | A |
| 84 | A | B | | A | B | A |
| 85 | A | A | | B | A | A |
| 86 | A | B | | A | A | C |
| 87 | A | A | | A | A | B |
| 88 | A | A | | | | |
| 89 | A | B | | | | |
| 90 | B | B | | | | B |
| 91 | D | B | | | | |
| 92 | B | A | | | | D |
| 93 | B | A | | | | C |
| 94 | A | A | | B | B | D |
| 95 | B | B | | | | B |
| 96 | B | C | | | | |
| 97 | C | C | | | | |

R-1 and R-2 are selected reference compounds included for comparison.

What is claimed is:

1. A compound of Formula (I): 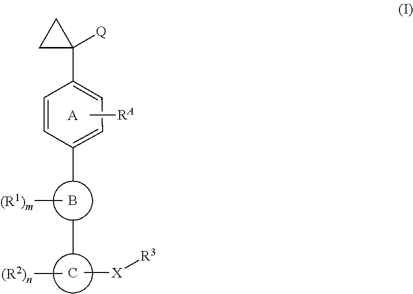
or pharmaceutically acceptable salts thereof, wherein:
R^{A} is hydrogen, C1-C6 alkyl, or C1-C6 alkoxy;
Ring B is 3-7 membered heterocyclylene or 5-6 membered heteroarylene;
Ring C is phenylene or 5-6 membered heteroarylene;
m is 0 or 1;
n is 0, 1, 2, or 3;
each R¹ is independently selected from halogen, C1-C6 alkyl, C1-C6 alkoxy, C1-C6 alkoxyalkyl, 3-7 membered heterocyclyl, and 5-6 membered heteroaryl; or two R¹ together with the atoms to which they are attached forms a 5-8 membered heterocyclyl or 5-6 membered heteroaryl;
each R² is independently selected from C1-C6 alkyl, C1-C6 haloalkyl, C1-C6 alkoxy, and halogen;
R³ is selected from:
(i) hydrogen,
(ii) C1-C6 alkyl optionally substituted with 1-2 independently selected R^{E},
(iii) C6-C10 aryl optionally substituted with hydroxyl,
(iv) C5-C10 cycloalkyl,
(v) 5-6 membered heteroaryl optionally substituted with phenyl, and
(vi) 5-6 membered heterocyclyl optionally substituted with phenyl;
each R^{E} is independently selected from:
(i) hydroxyl;
(ii) C1-C6 alkoxy,
(iii) phenyl optionally substituted with 1-3 substituents selected from C1-C6 alkyl, C1-C6 haloalkyl, C1-C6 alkoxyalkyl, C1-C6 alkoxy, hydroxyl, cyano, and halogen,
(iv) 5-10 membered heteroaryl optionally substituted with C1-C6 alkyl, and
(v) C3-C10 cycloalkyl;
Q is selected from:
(i) C1-C6 hydroxyalkyl,
(ii) —C(═O)OH,
(iii) —S(═O)₂OH,
(iv) cyano,
(v) —S(O)₂NR^{A1}R^{B1},
(vi) —NR^{C}S(O)₂NR^{A1}R^{B1},
(vii) —C(═O)C1-C6 alkoxy optionally substituted with C1-C6 alkoxy optionally substituted with —S(═O)₂NR^{C}R^{D}; or 5-6 membered heterocyclyl optionally substituted with C1-C6 alkyl;
(viii) —C(═O)C6-C10 aryloxy;
(ix) —C(═O)-(5-6 membered heterocyclyloxy) optionally substituted with 1-4 substituents selected from C1-C6 alkyl, hydroxyl, and —C(═O)OH,
(x) —C(═O)NR^{A2}R^{B2},
(xi) C1-C6 haloalkyl optionally substituted with hydroxyl,
(xii) 5-6 membered heteroaryl optionally substituted with hydroxyl, and
(xiii) —C(═O)NHS(═O)₂R^{F};
X is selected from —(CH₂)ₓNR^{G}C(═O)O—, —(CH₂)ₓOC(═O)NR^{G}—, —(CH₂)ₓNR^{G}, or —CH(OH)—; R^{A1} and R^{B1} are independently selected from hydrogen and C1-C6 alkyl optionally substituted with hydroxyl;
R^{A2} and R^{B2} are independently selected from hydrogen and C1-C6 alkyl optionally substituted with hydroxyl or —S(═O)₂NH₂;
R^{C} and R^{D} are independently selected from hydrogen and C1-C6 alkyl;
R^{F} is selected from:
(i) C1-C6 alkyl optionally substituted with 1-3 substituents selected from:
(a) hydroxyl,
(b) halogen,
(c) cyano,
(d) C1-C6 alkoxy,
(e) 4-6-membered heterocyclyl optionally substituted with C1-C6 alkyl,
(f) —C(═O)OH,
(g) —OC(═O)C1-C6 alkyl,
(h) —S(═O)₂C1-C6 alkyl,
(i) —C(═O)NR^{A1}R^{B1},
(j) —NR^{C}R^{D},
(k) —C(═O)C1-C6 aryloxy,
(l) C3-C6 cycloalkyl optionally substituted with —C(═O)OH, C1-C6 hydroxyalkyl, —C(═O)NR^{A1}R^{B1}, or —C(═O)C1-C6 alkoxy, and
(m) —C(═O)C1-C6 alkoxy optionally substituted with —C(═O)C1-C6 alkyl or —OC(═O)C1-C6 alkyl,
(ii) C2-C6 alkenyl,
(iii) C1-C6 haloalkyl,
(iv) phenyl,
(v) C3-C6 cycloalkyl optionally substituted with
(a) C1-C6 alkyl optionally substituted with 1-3 substituents selected from hydroxyl, —C(═O)OH, —C(═O)NR^{A1}R^{B1}, or —C(═O)C1-C6 alkoxy,
(b) cyano,
(c) —C(═O)OH,
(d) —C(═O)NR^{C}R^{D}, or
(e) —C(═O)C1-C6 alkoxy optionally substituted with hydroxyl or —C(═O)C1-C6 alkyl, or
(f) —NR^{C}R^{D}, and
(vi) —NR^{A3}R^{B3};
R^{A3} and R^{B3} are independently selected from hydrogen and C1-C6 alkyl;
R^{G} is H or C1-C3 alkyl;
and x is 0 or 1.

2. The compound of claim 1, wherein when Ring B is pyridyl, Ring B and Ring C are connected via a carbon atom of Ring C.

3. The compound of claim 1 or 2, wherein Ring B is 3-7 membered heterocyclylene.

4. The compound of any one of claims 1-3, wherein Ring B is selected from the group consisting of azetidinyl, pyrrolidinyl, dihydropyrrolyl, piperidinyl, piperazinyl, and azaspiro[3.3]heptanyl.

5. The compound of any one of claims 1-4, wherein Ring B is selected from the group consisting of 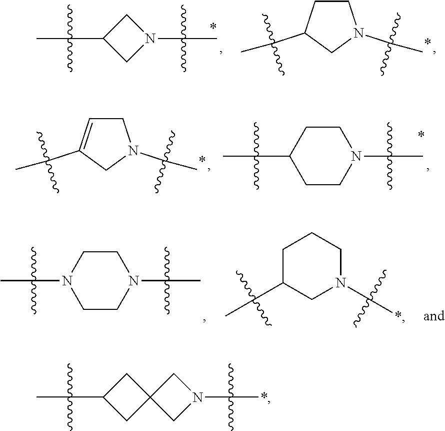
wherein “*” indicates the attachment point to Ring A.

6. The compound of any one of claims 1-5, wherein Ring B is 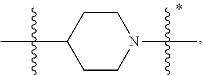
wherein “*” indicates the attachment point to Ring A.

7. The compound of any one of claims 1-6, wherein Ring C is 5-6 membered heteroarylene.

8. The compound of any one of claims 1-7, wherein Ring C is 5 membered heteroarylene selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl.

9. The compound of any one of claims 1-8, wherein Ring C is selected from the group consisting of 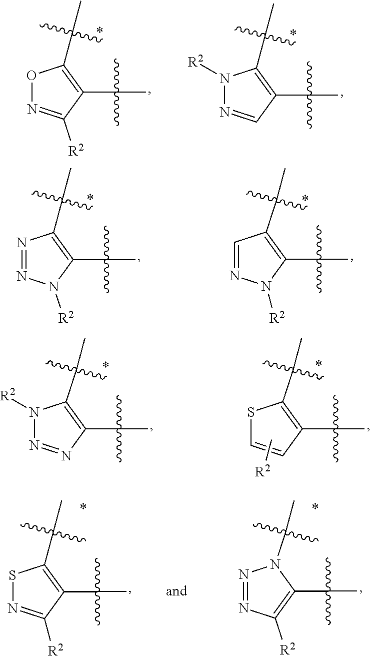
wherein “*” indicates the attachment point to Ring B.

10. The compound of any one of claim 1-9, wherein Ring C is 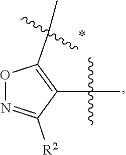
wherein “*” indicates the attachment point to Ring B.

11. The compound of any one of claims 1-7, wherein Ring C is phenylene.

12. The compound of any one of claims 1-11, wherein m is 0.

13. The compound of any one of claims 1-12, wherein n is 1.

14. The compound of any one of claims 1-11 and 13, wherein each R¹ is an independently selected C1-C6 alkyl.

15. The compound of any one of claims 1-14, wherein at least one R² is C1-C6 alkyl.

16. The compound of any one of claims 1-15, wherein at least one R² is methyl.

17. The compound of any one of claims 1-16, wherein R³ is C1-C6 alkyl optionally substituted with 1-2 R^{E}.

18. The compound of any one of claims 1-17, wherein at least one R^{E} is phenyl optionally substituted with 1-3 substituents selected from C1-C6 alkyl, C1-C6 haloalkyl, C1-C6 alkoxyalkyl, C1-C6 alkoxy, hydroxyl, cyano, or halogen.

19. The compound of any one of claims 1-18, wherein R³ is 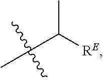
wherein R^{E} is phenyl optionally substituted with 1-3 substituents selected from C1-C3 alkyl, C1-C3 haloalkyl, C1-C3 alkoxyalkyl, C1-C63 alkoxy, hydroxyl, cyano, or halogen.

20. The compound of any one of claims 1-19, wherein R³ is

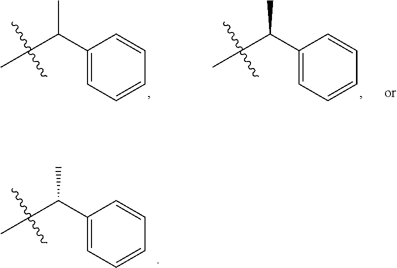

21. The compound of any one of claims 1-20, wherein Q is selected from —C(═O)OH; —S(═O)₂OH; 5-6 membered heteroaryl optionally substituted with hydroxyl; and —C(═O)NHS(═O)₂R^{F}.

22. The compound of any one of claims 1-21, wherein Q is —C(═O)OH or —C(═O)NHS(═O)₂R^{F}.

23. The compound of any one of claims 1-22, wherein Q is —C(═O)NHS(═O)₂R^{F}.

24. The compound of any one of claims 1-24, wherein R^{F} is C1-C6 alkyl optionally substituted with 1-3 substituents selected from hydroxyl; halogen; cyano; C1-C6 alkoxy; 4-6-membered heterocyclyl optionally substituted with C1-C6 alkyl; —C(═O)OH; —OC(═O)C1-C6 alkyl; —S(═O)₂C1-C6 alkyl; —C(═O)NR^{A1}R^{B1}; —NR^{C}R^{D}; —C(═O)C1-C6 aryloxy; C3-C6 cycloalkyl optionally substituted with —C(═O)OH, cyano, C1-C6 hydroxyalkyl, —C(═O)NR^{A1}R^{B1}, or —C(═O)C1-C6 alkoxy optionally substituted with hydroxyl or —C(═O)C1-C6 alkyl.

25. The compound of any one of claims 1-24, wherein R^{F} is C1-C6 alkyl optionally substituted with C(═O)OH or —C(═O)C1-C6 alkoxy.

26. The compound of any one of claims 1-23, wherein R^{F} is C2-C6 alkenyl.

27. The compound of any one of claims 1-22, wherein Q is —C(═O)OH.

28. The compound of any one of claims 1-22, wherein Q is —NR^{A3}R^{B3}.

29. The compound of any one of claims 1-28, wherein X is —(CH₂)ₓNR^{G}C(═O)O—.

30. The compound of any one of claims 1-29, wherein X is —NHC(═O)O—.

31. The compound of any one of claims 1-28, wherein X is —(CH₂)ₓOC(═O)NR^{G}—.

32. The compound of any one of claims 1-28 or 31, wherein X is —OC(═O)NH—.

33. The compound of any one of claims 1-28, wherein X is —(CH₂)ₓNR^{G}—.

34. The compound of any one of claims 1-28 or 33, wherein X is —NH—.

35. The compound of any one of claims 1-28, wherein X is —CH(OH)—.

36. The compound of claim 1, wherein Formula (I) is Formula (I-a): 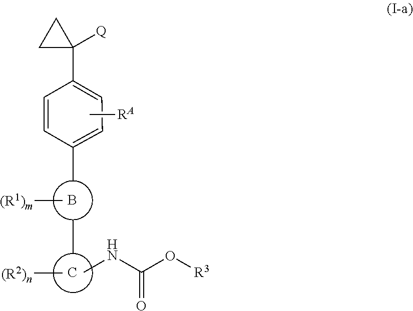
or pharmaceutically acceptable salts thereof.

37. The compound of claim 1, wherein Formula (I) is Formula (I-b): 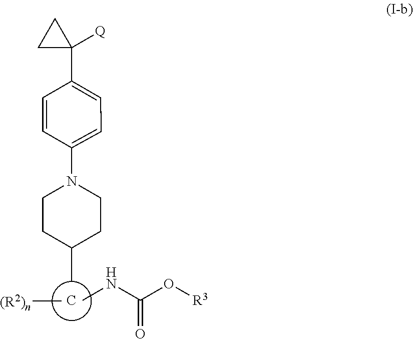
or pharmaceutically acceptable salts thereof.

38. The compound of claim 1, wherein Formula (I) is Formula (I-c): 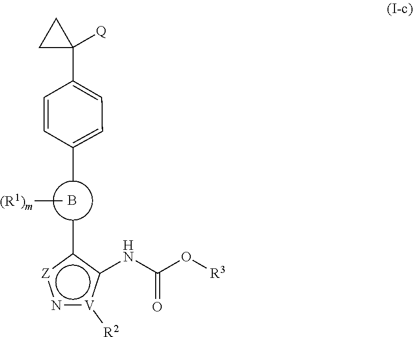
or pharmaceutically acceptable salts thereof, wherein Z is CH, N, O, or S; V is C or N; and wherein Z is O or S when V is C.

39. The compound of claim 1, wherein Formula (I) is Formula (I-d): 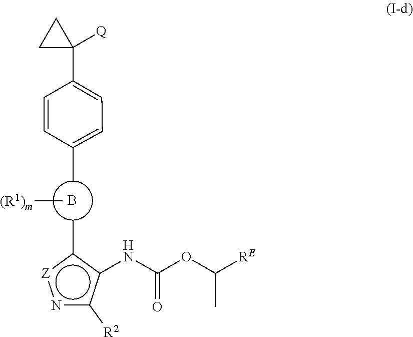
or pharmaceutically acceptable salts thereof, wherein Q is —CO₂H or —C(═O)NHS(═O)₂R^{F}; and Z is 0 or S.

40. The compound of claim 1, wherein Formula (I) is Formula (I-e): 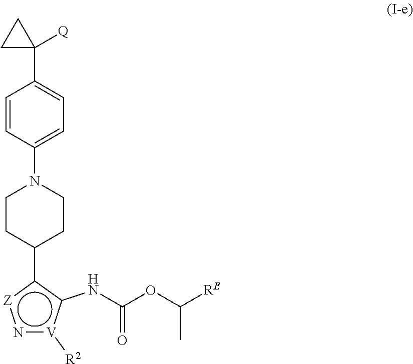
or pharmaceutically acceptable salts thereof,
wherein Q is —CO₂H or —C(═O)NHS(═O)₂R^{F}; Z is CH, N, O, or S; V is C or N; and wherein Z is 0 or S when V is C.

41. The compound of claim 1, wherein Formula (I) is Formula (I-f): 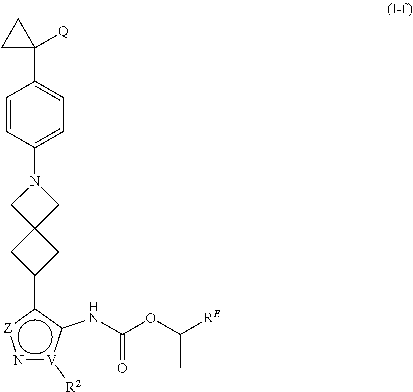
or pharmaceutically acceptable salts thereof,
wherein Q is —CO₂H or —C(═O)NHS(═O)₂R^{F}; Z is CH, N, O, or S; V is C or N; and wherein Z is 0 or S when V is C.

42. A compound selected from the group consisting of compounds in Table A, or a pharmaceutically acceptable salt of any of the foregoing.

43. A pharmaceutical composition comprising a compound of any one of claims 1-42, or a pharmaceutically acceptable salt thereof and a pharmaceutically acceptable carrier.

44. A method of treating a fibrotic disease in a subject in need thereof, comprising administering to the subject a therapeutically effective amount of a compound of any one of claims 1-42, or a pharmaceutically acceptable salt thereof, or the pharmaceutical composition of claim 43.
